(19) United States
(12) Patent Application Publication
(10) Pub. No.: US 2005/0244863 A1
(43) Pub. Date: Nov. 3, 2005

(54) Molecular arrays and single molecule detection

(75) Inventor: Kalim Mir, Oxford (GB)

Correspondence Address: PALMER & DODGE, LLP;KATHLEEN M. WILLIAMS, 111 HUNTINGTON AVENUE, BOSTON, MA, 02199, US

(73) Assignee: The Chancellor, Master and Scholars of The University of Oxford

(21) Appl. No.: 11/085,679

(22) Filed: Mar. 21, 2005

Related U.S. Application Data
(63) Continuation of application No. PCT/GB03/04041, filed on Sep. 19, 2003.

(30) Foreign Application Priority Data
Sep. 19, 2002 (GB) 0221792.5
Sep. 26, 2002 (GB) 0222412.9

(51) Int. Cl. C12Q 1/68; G01N 33/53; C12M 1/34

(52) U.S. Cl. 435/6; 435/7.1; 435/287.2; 427/2.11

(57) ABSTRACT

Methods are provided for producing a molecular array comprising a plurality of molecules immobilized to a solid substrate at a density which allows individual immobilized molecules to be individually resolved, wherein each individual molecule in the array is spatially addressable and the identity of each molecule is known or determined prior to immobilisation. The use of spatially addressable low density molecular arrays in single molecule detection techniques is also provided.

### FIELD OF THE INVENTION

The present invention relates to single molecule analytical approaches which are performed using molecular arrays.

In particular, the single molecule analytical approaches according to the invention involve tagging schemes, the detection of labels/tags and the determination of the spatial coordinates of a single molecule on the array. The invention further involves the direct measurement of physico-chemical properties of individual molecules and their interaction with other molecules. The use of the invention in a number of methods is described including SNP typing, haplotyping, gene expression analysis, proteomics and sequence determination, where the invention is particularly relevant to ultra-fast, parallel DNA sequencing which is applicable to the sequencing of whole genomes.

### BACKGROUND TO THE INVENTION

The analytical methods generally in use today involve analysing the reactions of molecules in bulk. Although bulk or ensemble approaches have in the past proved useful, culminating in an explosion in our understanding of molecular biology and recently to the sequencing of the human genome, there are barriers to future progress in a number of directions. The results generated by bulk analysis are an average of millions of individual molecular reactions where multiple events, multi-step events and variations from the average cannot be resolved and detection methods that are adapted for high frequency events are insensitive to rare events.

The bulk nature of conventional methods does not allow access to specific characteristics of individual molecules. One example in genetic analysis is the need to obtain genetic phase or haplotype information—the specific alleles associated with each chromosome. Bulk analysis cannot resolve haplotypes in a heterozygotic sample. The currently available molecular biology techniques, for this, such as allele-specific or single molecule PCR are difficult to optimise and apply on a large scale.

Bulk analysis typically requires a large amount of sample material. For example, Microarray gene expression analysis using unamplified cDNA target typically requires 10⁶ cells or 100 micrograms of tissue. Furthermore, neither expression analysis nor analysis of genetic variation can be performed directly on material obtained from a single cell which would be advantageous in a number of cases e.g. analysis of MRNA from cells in early development or genomic DNA from sperm.

Furthermore, it would be highly desirable if the amplification processes that are required before most biological or genetic analysis can be avoided. This includes amplification of molecules by cloning and the Polymerase chain reaction (PCR). The need is particularly acute in the large scale analysis of SNPs. The cost of performing SNP detection reactions on the scale required for high-throughput analysis of polymorphisms in a population is prohibitive if each reaction has to be conducted separately, or if only a limited multiplexing possibility exists. The need to design primers and perform PCR on a large number of SNP sites presents a major bottleneck, DNA pooling is a solution for some aspects of genetic analysis but accurate allele frequencies must be obtained.

Sequencing the human genome for the first time took more than ten years and hundreds of millions of dollars. Although this was achieved by use of Sanger-dideoxy method (Sanger F, Nicklen S, Coulson AR DNA sequencing with chain-terminating inhibitors. Proc Natl Acad Sci USA. 1977 December;74(12):5463-7), the methods involved are inherently slow and costly relying on electrophoresis which is slow, has limited separation range and not amenable to high degrees of parallelism.

Now, however, the need for large scale re-sequencing of individual human genomes and de novo sequencing in pathogens and model organisms require cheaper and faster alternatives to be developed. Recently, several methods that would avoid gel electrophoresis, cloning or the Polymerase-chain reaction (PCR) have been suggested (Pray L, A cheap personal genome? The Scientist, Daily News Oct. 4, 2002). One idea, “sequencing by synthesis” (SbS) which is attracting wide interest, involves the identification of each nucleotide immediately following its incorporation by polymerase into an extending DNA strand. Today, one SbS approach, pyrosequencing, is widely used for SNP (single-nucleotide polymorphism) typing (Ronaghi M, Uhlen M, Nyren P. A sequencing method based on real-time pyrophosphate. Science. 1998 Jul. 17;281(5375):363, 365.). In this case, the detection is based on pyrophosphate (PPi) release, its conversion to ATP, and the production of visible light by s firefly luciferase. However, because the signal is diffusible, pyrosequencing cannot take advantage of the massive degree of parallelism that becomes available when surface immobilised reactions are analysed.

Increasing read-lengths beyond those currently available would be highly useful. Moreover, it would be advantageous if sequencing runs can be on the scale of genomes, at least small genomes or whole genes or if thousands or millions of DNA fragments could be sequenced in parallel. It would also be useful if the confidence in the sequence information that is obtained could be increased. It would also be useful if the underlying haplotype information of the sequence could be retained. These facilities would aid the task of functional genomics by enabling genotype-phenotype correlations to be obtained at an unprecedented resolution and scale and would be widely applicable to disease genetics. If large amounts of data can be handled efficiently, sequencing would offer a number of advantages over typing SNPS. It would also have wide applications as a means for determining the identity of a molecule.

Array technology offers massive parallelization, but present implementations are limited by the constraints of bulk analysis. Array re-sequencing Patil N, et al. Blocks of limited haplotype diversity revealed by high-resolution scanning of human chromosome 21. Science. 2001 Nov. 23;294(5547):1719-23) offers high parallism but is highly complex.

Furthermore, following the sequencing of the human genome the emphasis has shifted to the analysis of gene products, namely RNA and particularly proteins. The methods available for protein analysis are not typically available in a highly parallel format. 2-D gel electrophoresis has been traditionally used to analyse populations of proteins but this method is difficult to implement particularly as it relies on gel electrophoresis. Recently there are efforts towards developing protein microarrays. However, to date there is no established method for conducting proteomics in a rapid and sensitive manner, which is widely applicable.

Furthermore, sensitive, high-throughput methods are needed for analysing the interactions of proteins with small molecules.

New techniques are being developed that forgo traditional ‘bulk’ biochemical methods that analyse the average signal from an ensemble of molecules and instead examine single molecules. A single binding event or reaction can be amplified by RCA (Lizardi P M, Huang X, Zhu Z, Bray-Ward P, Thomas D C, and Ward D C. 1998. Mutation detection and single-molecule counting using isothermal rolling-circle amplification. Nat Genet 19 225-32.63 Schultz S, Smith D R, Mock J J, Schultz D A. Single-target molecule detection with nonbleaching multicolor optical immunolabels. Proc Natl Acad Sci USA. 2000 Feb. 1;97(3):996-1001. AND Oldenburg S J, Genick C C, Clark K A, Schultz D A. Base pair mismatch recognition using plasmon resonant particle labels. Anal Biochem. 2002 Oct. 1;309(1):109-116) or by labeling with nanoparticles and a number of techniques have been developed that can start with a single molecule and then do PCR amplification; these include MPSS (Brenner S, Williams S R, Vermaas E H, Storck T, Moon K, McCollum C, Mao J I, Luo S, Kirchner J J, Eletr S, DuBridge R B, Burcham T, Albrecht G. In vitro cloning of complex mixtures of DNA on microbeads: physical separation of differentially expressed cDNAs. Proc Natl Acad Sci U S A. 2000 Feb. 15;97(4):1665-70), Colony PCR (Mitra R D, Church G M. In situ localized amplification and contact replication of many individual DNA molecules. Nucleic Acids Res. 1999 Dec. 15;27(24):e34 AND Mitra R D, Butty V L, Shendure J, Williams B R, Housman D E, Church G M. Digital genotyping and haplotyping with polymerase colonies. Proc Natl Acad Sci USA 2003 May 13;100(10):5926-31. Epub 2003 May 2 AND Mitra R D, Shendure J, Olejnik J, Edyta-Krzymanska-Olejnik, Church G M. Fluorescent in situ sequencing on polymerase colonies. Anal Biochem. 2003 Sep. 1;320(1):55-65) and Digital PCR (Vogelstein B, Kinzler K W. Digital PCR. Proc Natl Acad Sci USA. 1999 Aug. 3;96(16):9236-41). A commercial SNP typing system based on Fluorescence Correlation Spectroscopy (FCS) of multi-labelled single molecules has recently been introduced⁶⁵ (Olympus/Evotec OA). However it is not a significant departure from other homogeneous techniques because even though single molecules are detected as they pass through the small focus volume of a laser, the assay strategy still retains the PCR step. Single molecule methods using optical laser-trapping have been developed to study the transcription of immobilised RNA polymerase molecules (Yin et al., 1995, Science 270:1653-56).

The signal from a single molecule does not need to be amplified to be detected, as a single fluorophore label emits enough photons to be detected if background noise is sufficiently suppressed.

In recent years methods have been developed for detecting and analysing individual molecules labeled with single dye molecules on surfaces or in solution. Individual ATP turnover by single myosin molecules has been visualised using evanescent wave excitation (Funatsu et al., 1995, Nature 374: 555-59). Moreover, analysis has been performed on single molecules in unamplifled genomic DNA (Castro A. and Williams J G K, 1997, Anal. Chem. 69:3915-3920). Coincident single molecule detection of two PNA probes each labeled with a single fluorophore, as they hybridise to proximal sequences on genomic DNA passing through a sheath flow provided the specificity to detect an unamplified, single-copy target DNA molecule within a complex genomic background in a homogeneous assay. Methods for sequencing on single molecules are now under development (Braslavsky I, Hebert B, Kartalov E, Quake S R. Sequence information can be obtained from single DNA molecules. Proc Natl Acad Sci USA. 2003 Apr. 1;100(7):39604.).

The methods described so far detect fluorescent signals from single molecules but do not visualize the molecules themselves. Other techniques visualize the DNA as a polymer on a surface. DNA polymers on a surface have been probed at SNP sites using tagged probes that can be detected by the AFM and by fluorescent probes.

Technologies that permit the elimination of PCR, such as those based on single molecule examination would increase throughput and bring down costs but are faced with the formidable complexity of the human genome which impacts the specificity with which a desired locus can be targeted. Nevertheless, encouragement can be found in recent results from generic PCR which suggest that the problems of genome complexity can be substantially overcome by suppressing repetitive sequences.

Such whole human genome sequencing would be able to access disease causing mutations directly including those to which the common SNPs do not associate through linkage disequilibrium. It could also open up an era of personalized medicine in which health management is informed by an individual's genomic sequence.

The prior art pertaining to molecular arrays is not specifically applicable to the analysis of single molecules. Where the term “molecule” is mentioned, it is obvious that the methods are not relevant to single molecule techniques because strategies for detecting single molecules are not described.

In general, the methods of the prior art do not examine single molecules individually but examine large homogeneous populations of substantially identical molecules, wherein the signal which is used to identify a label originates from a bulk population of molecules rather than an individual molecule. Conventional usage does not generally facilitate this distinction: phrases such a “a molecule” or “a sample molecule” as used in the prior art generally do not refer to an individual molecule considered separately or in isolation from other molecules, including separately from other molecules of identical composition and structure, but to populations comprising millions or more molecules of identical structure. Invariably, investigators are not working with samples consisting of single molecules but rather with samples comprising a plurality of identical molecules. In particular, even where these investigators do not (as is consistent with conventional usage) explicitly note this point, they take measures which would apply only to samples of pluralities of identical molecules, and do not take measures associated with working with single molecules.

In WO02/074988 the present inventor described a method for preparing and using single molecule arrays which are comprised of individually resolvable, spatially addressable molecules, the identity of which is known or determined prior to immobilisation.

### SUMMARY OF THE INVENTION

The present invention overcomes the above-mentioned practical limitations associated with bulk analysis. This is achieved by the precision, richness of information, speed and throughput that is obtained by taking analysis to the level of single molecules.

To date single molecule analysis has only been conducted on very simple samples but presently the need to apply tests on larger scales is increasing. An important aspect of any single molecule technique for rapid analysis of large numbers of molecules will be a system for sorting/organising individual molecules and tracking/following individual events on them in parallel.

The approach of the present invention is set apart from traditional bulk array technologies inter alia by the type of information it aims to acquire, information that is based on the analysis of single molecules as separate, individual entities. The low density signals would not be readable by instrumentation typically used for analysing the results of bulk. The manufacture of single molecule arrays of the invention requires special measures as described herein.

1. Arrays

Low Molecular Density

Arrays useful in the present invention can be produced by a method which comprises immobilising on a solid phase a plurality of molecules at a density which allows individual immobilised molecules to be individually resolved. Alternatively, said method comprises immobilising to a solid phase a plurality of defined molecules at a density which allows individual immobilised molecule to be individually resolved by a method of choice, wherein each individual molecule in the array is or can become spatially addressable.

High Molecular Density

Arrays may moreover be produced by a method which comprises:
(i) providing a molecular array comprising a plurality of molecules immobilised to a solid phase at a density such that individual immobilised molecules are not capable of being individually resolved; and
(ii) reducing the density of functional immobilised molecules in the array such that the remaining individual functional immobilised molecules are capable of being individually resolved

The method may also comprise:
(i) providing a molecular array comprising a plurality of defined spatially addressable molecules immobilised to a solid phase at a density such that individual immobilised molecules are not capable of being individually resolved by optical means or another method of choice; and
(ii) reducing the density of functional immobilised molecules in the array such that each remaining individual functional immobilised molecule is capable of being individually resolved.

According to the above embodiments the invention further provides a method for producing a double stranded nucleic acid array, whereby the sample that is arrayed is double stranded prior to arraying. The invention provides a method for producing a single stranded nucleic acid array, whereby the sample that is arrayed is single stranded prior to arraying.

Alternatively, according to the above embodiments the invention further provides a method for producing a double stranded nucleic acid array, whereby the sample that is arrayed is not double stranded prior to arraying but is made double stranded after arraying. The invention provides a method for producing a single stranded nucleic acid array, whereby the sample that is arrayed is not single stranded prior to arraying but is made single stranded after arraying.

Encoded Molecules

The present invention also provides a method for producing a molecular array comprising a plurality of molecules immobilised to a solid phase at a density which allows each individual immobilised molecule to be individually resolved, wherein the identity of each individual molecule is encoded and can be decoded, for example with reference to a look up table.

The present invention also relates to methods of arraying pluralities of nucleic acid molecules at low density where, although the identity of the nucleic acids may be unknown prior to immobilisation, the array is subsequently characterised by the use of encoded probes, such as tagged probes or by successive serial addition and/or removal of probes from a repertoire and then reconstructing the sequence identity from information about which of the probes interact with which of the immobilized nucleic acids.

In this embodiment the molecules are first placed randomly on the surface and the decoding process is carried out to make them spatially addressable i.e. to correlate an individual location on the array with the identity of the molecule at that particular location. This means that the molecules may be randomly distributed on the array, which is simpler, faster and cheaper way of putting the molecules on the surface as compared to in situ synthesis or spotting of spatially addressable arrays. The decoding process may involve methods known in the art such as Sequencing by synthesis. In a preferred embodiment the decoding process involves interacting the array with a repertoire of probes.

Thus, in a further aspect, the present invention provides a method for arraying a plurality of nucleic acid molecules which method comprises:
(i) contacting the plurality of nucleic acid molecules with a plurality of probes, each probe being labelled with a tag which indicates the identity of the probe, such that each molecule can be identified by detecting the probes bound to the molecule and determining the identity of the corresponding tags;
(ii) immobilising the plurality of nucleic acid molecules randomly to a solid substrate; and optionally
(iii) horizontalising and optionally straightening the molecules during or after immobilisation

The plurality of nucleic acids are immobilised at a density which allows individual molecules in the array to be individually resolved.

Horizontalisation is defined as the immobilsation of the DNA so that it is substantially in a parallel plane to the surface. This may be achieved by multiple interactions on the surface or by directional fluid flow. In most cases of horizontalisation it is preferable that the molecule is substantially straigtened, as can be assessed by far-field optical microscopy. An exception is where different regions of a DNA polymer are deliberately directed to particular spatial locations. This horizontalisation and straightening can also be described as combing but the processes used are different from those used in molecular combing.

In an alternative embodiment, the present invention provides a method for arraying a plurality of nucleic acid molecules which method comprises:
(i) immobilising the plurality of nucleic acid molecules randomly to a solid substrate;
(ii) optionally horizontalising and straightening the molecules during or after immobilization.
(iii) contacting the plurality of nucleic acid molecules with a one or a plurality of probes, each probe being labelled with a tag which indicates the identity of the probe, such that each immobilised molecule can be identified by detecting the probes bound to the molecule and determining the identity of the corresponding tags.
(iv) Optionally repeating step (iii) until the identity of molecule becomes substantially established

The plurality of nucleic acids are immobilised at a density which allows individual molecules in the array to be individually resolved.

In one specific embodiment the repertoire is of polynucleotides whose identity is decoded by the following steps:
i) adding to said array composition a first set of decoding nucleotides/probes
ii) at least one nucleotide of molecule that basepair with at least one nucleotide of at least one of said labelled decoding nucleotide/probe; and
iii) detecting the presence of said label at particular location
iv) Optionally removing said decoding probes and repeating steps i) and iii), wherein said nucleotide sequence being decoded will base-pair with a different nucleotide of a second set of decoding nucleotides/probes.
v) compiling the sequence to provide identity of molecule at particular array locations

The probes may be oligonucleotides which are shorter in length than the polynucleotides of the array.

In above embodiments upon determination of identity the immobilised molecules of the array become spatially addressable.

Iteration of this process can further sequence characterize the molecule until a full sequence is obtained as will be described below.

Once decoded the array can then be used for further investigations for example in mRNA quantitation.

Direct Arraying the Sample

In a further aspect, the present invention can be used more generally to produce low density arrays of molecules in a sample to enable characterization of the molecules in the sample under analysis. Thus the present invention also provides a method for producing a molecular array which method comprises immobilising to a solid phase a plurality of molecules present in a sample under analysis, wherein the plurality of molecules are immobilised at a density such that individual molecules in the sample can be individually resolved.

The plurality of molecules may comprise the genome, proteome, transcriptome or metabalome of a cell, tissue or organism. The resulting arrays may be used in genome or proteome analyses.

In a specific embodiment of the invention an array of capture molecules are spread onto a surface to form a primary array. This is followed by the formation of a secondary array by the addition of the sample molecules to the surface under conditions that the sample molecules interact with the primary array. For example, sticky ends are created on sample DNA (these may be optionally further recessed) and bind to probes randomly arrayed on the surface. Alternatively the surface may comprise a spatially random set of oligonucleotide capture probes which will bind to any regions of complementary sequence that may be accessible. Accessibility is induced in substantially double stranded target by partial denaturation (heating, pH etc) or by use of the RecA Protein. Alternatively, the sample may be substantially single stranded.

Linked mRNA-Protein Arrays

mRNA coding for protein of interest, a puromycin attached to the 3′ end of mRNA using a synthetic linker, the mRNA puromycin complex is subject to in vitro translation to generate the protein, the puromycin then links to the proteins. Hence a protein is linked to it's coding mRNA. A spatially addressable array is then made in which each molecular complex is individually resolvable or is individually functionalized so that it can be individually resolvable. Alternatively, this mRNA-protein complex is then spread onto a surface to produce a spatially random array in which each molecular complex is individually resolvable. This can then made addressable by binding of decoding sequences. A contiguous sequence length between 10 and 16 bases will in most cases be sufficient to identify the mRNA and thereby the protein uniquely. If the sequence is obtained from a particular position along the mRNA the sequence information required will be less. For example 10 or 11 bases of sequence information from the 3′ untranslated region will be sufficient. The sequence information can be obtained by any method known in the art, including Sequencing by Hybridisation and Sequencing by synthesis. In both cases a primer could be provided such as oligo dT which binds at the PolyA tail and primes synthesis of 10 bases of sequence information. In the case of Sequencing by Hybridisation the oligo dT will promote stacking hybridization of for example 6 mers which are differentially tagged. The characteristics and interactions of the protein can be probed by the methods of this invention.

Proteins Arrays

Spatially addressable arrays of proteins or polypetides can be made can be made in which each individual molecule is individually resolvable. Alternatively, spatially random protein array can be made and the molecules of the array made spatially addressable by binding of a repertoire of peptides or antibodies, affibodies, aptamers so that they can be identified.

Directing Different Loci on a Single Polymer Molecule to Different Spatial Locations

In one embodiment the immobilised molecules are present within discrete spatially addressable elements. In one such embodiment, a plurality of molecular species are present within one or more of the discrete spatially addressable elements and each molecular species in an element can be distinguished from other molecular species in the element by means of a label. In another embodiment the plurality of molecules are not distinguishable by a label but comprise a group of sequences, for example representing members of a gene family, according to which they may be distinguished. In a further preferred embodiment, the probes are oligonucleotides or polynucleotides and the molecules are provided as groups of molecules, members of each group of molecules are complementary (and thereby each able to hybridise) to a different site such as a locus of interest, within the target nucleic acid molecule and immobilised to the solid phase such that each group is spatially distinct from the other groups. In one such embodiment the spatially addressable elements are coincident with an electrical semi-conductor or conductor layer.

The present invention also provides a multiplexed array comprising a plurality of molecular arrays produced by the above methods of the inventions. Methods for producing such multiplexed arrays are also provided. The multiplexed arrays may be used in multiplexed analysis. The multiplexing can be of arrays in which molecules are spatially addressable or random.

Typically, in any of the above embodiments, the solid phase is a substantially flat solid substrate or a bead/particle/bar. “Solid phase”, as used herein, refers to any material which is isolatable from solutions and thus includes porous materials, gels and gel-covered materials. In one embodiment the solid-phase comprises microscopic particles which are placed on a planar solid surface and where preferably the microscopic particles are metallic or semiconductor particles.

In a particular embodiment, the solid phase comprises channels or capillaries within which the molecules are immobilised. Moreover, the molecular array can be formed on or in an optical fibre.

The molecular array can comprise nucleic acids which form secondary structures, said secondary structures facilitating or stabilising hybridisation or improving mismatch discrimination.

The array can be an array of anti-tags to which tags labeling a sample repertoire can be decoded.

The present invention also provides a molecular array obtained by the above methods.

Spreads: Spatially Random Arrays

A method for creating spatially random arrays whereby the sample is placed between two flat surfaces, optionally the surface is chemically derivatised and optionally the sample is exposed to electromagnetic (UV) irradiation, one surface is removed from the other by a lateral motion, optionally unadsorbed material is removed from the surfaces and optionally the surface undergoes further UV crosslinking. Random arrays are now created on both of the two flat surfaces.

The repertoire is preferably a repertoire of probes, for example a sample repertoire. Advantageously, the repertoire comprises nucleic acids, proteins and/or protein-nucleic acid hybrids.

Secondary Array

Secondary arrays can be created on a primary array. For example, if the primary array is an arrayed repertoire of probes, they can be used to capture a repertoire of sample molecules.

Spreads of Linearised Polymers

A method for creating spatially random arrays of linearised polymers whereby the sample is placed between two flat surfaces, optionally the surface is chemically derivatised, one surface is removed from the other by a lateral motion, optionally excess material is removed from the surfaces. Random arrays of linearised polymer are now created on both of the two flat surfaces. This method produces very good distributions of molecules where typically it is difficult to produce homogeneous molecular combing. The molecules of a secondary array can be straightened/linearised in this way.

2. Use of Arrays

The invention provides the use of a molecular array, for example as described herein, to perform single molecule analysis. In one embodiment said analysis can form part of a molecular assay.

The present invention further provides means to analyse the array of single molecules, wherein a physical, chemical or other property may be determined. For example, molecules which fluoresce at a certain tested wavelength can be directly sampled. This is particularly applicable where the repertoire is a repertoire created by in vitro evolution or SELEX experiments. The invention also provides techniques for measuring the physical properties of the molecules comprising the array or their interaction with various types of probes.

The present invention further provides a number of techniques for detecting interactions between sample molecules and the constituent molecules of molecular arrays.

Accordingly, the present invention provides the use of a molecular array, for example as described herein, in a method of identifying one or more array molecules which interact with a target.

The molecular array may also be used more generally in identifying compounds which interact with one or more molecules in the array. In this case the preferred targets would be small molecules, RNA molecules, proteins or genomic DNA.

Typically said methods comprise contacting the array with the sample and interrogating one or more individual immobilised molecules to determine whether a target molecule has bound.

Preferably interrogation is by an optical method such as a method selected from far-field optical methods, near-field optical methods, epi-fluorescence imaging, scanning confocal microscopy, two-photon microscopy, and total internal reflection microscopy. Other methods of microscopy, such as scanning probe microscopy and electron microscopy are also appropriate.

In one embodiment, the immobilised molecules are of the same chemical class as the target molecules. In another embodiment, the immobilised molecules are of a different chemical class to the target molecules.

In a preferred aspect, target molecules are genomic DNA or cDNA or mRNA. Accordingly, the molecular array may be used, for example in gene expression studies or the detection of single nucleotide polymorphisms (SNPs) in a sample of nucleic acids.

Thus in one preferred embodiment the immobilised molecules of the array and the target molecules are nucleic acids and the contacting step takes place under conditions which allow hybridisation of the immobilised molecules to the target molecules or the contacting step takes place under conditions which allow annealing and template (target) directed enzymatic processing of the immobilised molecules.

Sample nucleic acids can be fragmented prior to analysis. Large and/or complex samples, such as genomic samples, can be sorted prior to analysis e.g. according to chromosome by for example flow cytometry.

Sample complexity can be reduced by fragmenting the target and pre-hybridising it to C₀t=1 DNA. The samples DNA then undergoes whole genome amplification prior to analysis.

The single molecule methods allows the use of small samples and the detection of very small quantities of analyte in said samples—as low as a single molecule.

Particular applications of molecular arrays according to the invention, and of single molecule detection in techniques in general, are set forth herein. Particularly preferred uses include nucleic acid analysis, such as in SNP typing, sequencing and the like, in genetic and genomic analysis as well as uses for proteomics. These uses may be carried out in a large-scale format or in a compact biosensor device.

3. Specific Applications

The repertoires and arrays of the invention can be used to execute a number of different applications. These include SNP typing, gene expression analysis, sequencing and protein expression and characterisation.

SNP Typing

In a further aspect, the invention relates to a method for typing single nucleotide polymorphisms (SNPs) and mutations in nucleic acids, comprising the steps of:
a) providing a repertoire of probes complementary to one or more nucleic acids present in a sample, which nucleic acids may possess one or more polymorphisms, said repertoire being presented such that molecules may be individually resolved;
b) exposing the sample to the repertoire and allowing nucleic acids present in the sample to anneal to the probes at a desired stringency, and optionally further processing;
c) detecting binding events or the result of processing.

The detection of binding events may be aided by eluting the non-annealed/unprocessed nucleic acids from the repertoire and detecting individual hybridised/processed nucleic acid molecules. The processing includes enzyme reactions such as primer extension, single base extension, ligation, padlock probe ligation and rolling circle amplification.

In a one embodiment sequence is extended from primer. Extension may be of one base or a few bases (to characterisation of insertions/deletions, Indels).

In a preferred embodiment the repertoire of probes target SNPs that that “tag” the haplotypes of a given region of Linkage disequilibrium and leaving out SNPs that provide redundant information

Advantageously, the repertoire is presented as an array, which is preferably an array as described hereinbefore.

Haplotyping

The invention is moreover applicable to haplotyping, in which a multiallelic probe set is used to analyse each sample molecule in a population for two or more features simultaneously. For example, a first probe may be used to immobilise the sample nucleic acid to the solid phase, and optionally simultaneously to identify one polymorphism or mutation; and a second probe may be used to interact with the immobilised sample nucleic acid and detect a second polymorphism or mutation. Thus, the first probe (or biallelic probe set) is arrayed on the solid phase, and the second probe (or biallelic probe set) is provided in solution (or is also arrayed; see below). Further probes may be used to test further SNP sites along the DNA. polymer as required. Thus, the method of the invention may comprise a further step of hybridising the sample nucleic acid with one or more further probes in solution.

The signals generated by the first and second probe sets may be differentiated, for example, by the use of differentiable signal molecules such as fluorophores emitting at different wavelengths, as described in more detail below. Moreover, the signals may be differentiable based on their location on the solid phase. To aid detection of the location of signal along the molecule, molecules may be stretched out by methods known in the art.

Within a probe set, the signals generated by two or more allelic probes may be differentiated, for example, by the use of differentiable signal molecules such as fluorophores emitting at different wavelengths, as described in more detail below. Moreover, the signals may be differentiable based on their location on the solid phase.

In a further preferred embodiment, the probes are oligonucleotides or polynucleotides and the molecules are provided as groups of molecules, each group of molecules complementary (and thereby each able to hybridise) to a different site such as a locus of interest, or a different variant such as SNP allele, within a target nucleic acid molecule and immobilised to the solid phase such that each group is spatially distinct from the other groups. In one such embodiment the spatially addressable elements are coincident with an electrical semi-conductor or conductor layer.

A method for haplotyping which involves the detection of the identity of SNP alleles along a single DNA polymer by binding to probes whose identity is linked to their spatial location on a surface. The spatial location of signal provides the read-out of the technique. This approach is particularly advantageous as it enables in situ synthesis of probes and does not require separate oligonucleotides to be synthesised.

In one embodiment spatial coordinates occupied by the single DNA polymer is detected by fluorescence staining. In another embodiment the spatial coordinates occupied by the testing electrical continuity between electrodes carrying each of the allele combinations virtue of it's formation of a circuit across a pair of electrodes, bearing probes testing contiguous SNP sites on the sample molecules, by the spanning of the electrodes.

A method for creating electrodes by DNA directed coating of the microarray spots with a conducting material and a method of creating a nanowire by directed DNA spanning two or more spatially addressable probes.

Analysing Sequence Repeats

In a further embodiment, the invention provides a method for determining the number of sequence repeats in a sample nucleic acid, comprising the steps of:
a) providing one or more probes complementary to one or more nucleic acids present in a sample, which nucleic acids may possess one or more sequence repeats, said probes being presented such that molecules may be individually resolved;
b) annealing a sample of nucleic acid comprising the repeats to the probes
c) contacting the nucleic acids with labelled probes complementary to said sequence repeats optionally in the presence of DNA ligase, or alternatively contacting with nucleotides (a mixture of labelled and nonlabelled at a ratio that would enable only one labelled incorporation per repeat) and a polymerase; and
d) determining the number of repeats present on each sample nucleic acid by individual assessment of the number of labels incorporated into each molecule, such as by measuring the brightness of the signal produced by the labels; wherein in a preferred embodiment signal is only processed from molecules to which a second solution oligonucleotide labelled with a different label is also incorporated.

The results may be analysed in terms of intensity ratios of the repeat probes labelled with first colour and the second probe labelled with a second colour.

Advantageously, the repertoire is presented as an array, which is preferably an array as described hereinbefore.

mRNA Analysis

The invention moreover provides a method for analysing the expression of one or more genes in a sample, comprising the steps of:
a) providing a repertoire of probes complementary to one or more nucleic acids present in a sample, said repertoire being presented such that molecules may be individually resolved;
b) hybridising a sample comprising said nucleic acids to the probes;
c) determining the nature and quantity of each individual nucleic acid species present in the sample by counting single molecules which are hybridised to the probes.

In some cases the individual molecule may be further probed by sequences that would differentiate alternative transcripts or different members of a gene family.

Advantageously, the repertoire is presented as an array, which is preferably an array as described herein.

Preferably, the probe repertoire comprises a plurality of probes of each given specificity, thus permitting capture of more than one of each species of nucleic acid molecule in the sample. This enables accurate quantitation of expression levels by single molecule counting.

Advantageously one or more mRNA populations, each population differently labelled so that its molecules can be distinguished from molecules from another population are interacted simultaneously with the repertoire of probes. This enables easy side-by-side comparisons of the differential expression of genes between the different populations analysed.

Advantageously the probes are designed to hybridise to specific positions on a mRNA molecule from the following: polydenlyation signal, (e.g. AAUAAA), Poly A tail, 5′ cap or sequence clamped to the 5′ or 3′ end of the molecules of the mRNA population.

In an alternative embodiment a sample mRNA population is spatially randomly arrayed and the identity of the sequence is determined by the hybridisation of decoding probes to reveal the identity of the mRNA. Hence gene expression analysis can be conducted by compiling the quantity of molecules of each individual identity present on the surface.

Sequencing

In a still further aspect, the invention relates to a method for determining the sequence of one or more target DNA molecules. Such a method is applicable, for example, in a method for fingerprinting a nucleic acid sample. Moreover the method may be applied to complete or partial sequence determination of a nucleic acid molecule or population of molecules.

Sequencing on Linearised Molecules

Genomic sequence would have much greater utility if haplotype information (the association of alleles along a single DNA molecule derived from a single parental chromosome) could be obtained over a long range. This is possible by following sequencing on a single molecule and more preferably where the single molecule is linearised on a surface enabling multiple sites from which sequence information is obtained are resolved. Here each template molecule is straitened to provide a linear display of sequence along its length. and allowing multiples foci along its length to be resolved.

Capture and Sequence

Thus, the invention provides a method for determining the complete or partial sequence of a target nucleic acid, comprising the steps of:
a) providing a repertoire of probes complementary to one or more nucleic acids present in a sample, said first repertoire being presented such that molecules may be individually resolved;
b) hybridising a sample comprising a target nucleic acid to the probes;
c) hybridising one or more further probes of defined sequence to the target nucleic acid; and
d) detecting the binding of individual further probes to the target nucleic acid.
e) optionally repeating steps c-d
f) reconstructing the sequence of the target polynucleotide

Advantageously, the further probes are labelled with labels which are differentiable, such as different fluorophores.

Advantageously, the repertoire is presented as an array, which is preferably an array as described hereinbefore.

General sequencing can be conducted by providing a complete repertoire of probes of a given length. More directed sequencing can be conducted by providing a complete repertoire of probes covering for example a repertoire of SNPs.

Direct Immobilization of Target

The present invention also provides methods for determining all or part of the sequence of a target nucleic acid molecule which does not require immobilised arrays of probe molecules for capturing the target. Instead, the target molecule is immobilized to a solid phase, preferably being horizontalised and straightened. Then probes are used to interrogate the immobilised target. The immobilised target may be a repertoire of oligonucleotides. Each oligonucleotide molecule within the repertoire is then sequenced by hybridisation of a repertoire of shorter oligos. This sequenced and now spatially addressable immobilised repertoire can then be used for further array experiments e.g. SbH, gene expression analysis or as primers for Sequencing by synthesis.

The further probes may act as primers for a variety of other template directed enzymatic reactions for example, the synthesis of a complementary DNA strand by the use of DNA polymerase and the provision of nucleotides. This is compatible with further sequence characterization by providing fluorescently tagged nucleotides whose incorporations are monitored in a way that enables the identity of each nucleotide to be determined.

In an advantageous embodiment, target nucleic acids are captured and/or immobilised on the solid phase surface at multiple points, which allows the molecule to be arranged horizontally on the surface and optionally sites on the target where immobilisation reaction occurs are in such locations that the target molecule is elongated. In a further embodiment the molecule is attached by a single point and physical measures are taken to horizontalise it. Hybridisation of further probes may then be determined according to position as well as or instead of according to differences in label.

In particular, the probes may be encoded i.e. labelled with tags whose identity can be readily determined, such as by using single molecule detection techniques. Detection is generally used to determine the position of the tagged probes with respect to the ends of the target molecules or other landmarks. The use of multiple probes then allows a sequence to be built up. When multiple copies of each target species is present then overlapping sequence information that is obtained can be used to build up the sequence by ‘Sequencing by Hybridisation’ methods known in the art.

Accordingly, the present invention provides a method for determining the sequence of all or part of a target nucleic acid molecule which method comprises:
(i) immobilising the target molecule to a solid phase at one or more points such that the molecule is substantially horizontal with respect to the surface of the solid phase;
(ii) straightening the target molecule during or after immobilisation;
(iii) contacting the target molecule with a nucleic acid probe of known sequence; and
(iv) determining the position within the target molecule to which the probe hybridises.
(v) repeating steps (iii) to (iv) as necessary, and
(vi) reconstructing the sequence of the target molecule.

The target may be immobilized at one point but linearised by fluid flow.

Preferably the target molecule is contacted with a plurality of probes,

In one embodiment target molecule is contacted with all of the plurality of probes substantially simultaneously. Preferably each probe is encoded, for example labelled with a different detectable label or tag.

Alternatively, the target molecule may be contacted sequentially with each of the plurality of probes.

Accordingly, method in which each of the plurality of labeled probes are successively hybridized to the immobilized nucleic acid and a record of those that hybridise to each molecule can be used to identify or re-assemble the sequence of the immobilized molecule.

In one embodiment the complete set of oligonucleotides of a given length are provided as probes.

In one embodiment different sub-sets of probes are provided in different experiments but the probes within each sub-set are differentially labeled. A method in which sub-sets are grouped according to their Tm

In one embodiment each probe or its label/tag is removed from the target molecule prior to contacting the target molecule with a different probe. Typically, the probes are removed by heating, modifying the salt concentration or pH, or by applying an appropriately biased electrical field.

In one embodiment the target is substantially a double stranded molecule and the probes are LNA or PNA and bind by strand invasion under appropriate conditions. In another embodiment the probes are Padlock Probes which bind to the target under appropriate conditions and become fixed to the target by a ligation reaction. In another embodiment RecA mediates the binding of the probes to a substantially double stranded molecule.

In another embodiment the target is substantially single stranded and is made accessible for subsequent hybridisation by stretching out/straightening, which may be achieved by capillary forces acting on the target in solution.

In one embodiment, where it is desired to determine the sequence of single-stranded molecules, the target nucleic acid molecule is a double-stranded molecule and is derived from such a single-stranded nucleic acid molecule of interest by synthesising a complementary strand to said single-stranded nucleic acid.

The present invention also provides a method for determining the sequence of all or part of a target single-stranded nucleic acid molecule which method comprises:
(i) contacting the target molecule with a plurality of nucleic acid probes of known sequence, each probes being labelled with a different detectable label; and
(ii) ligating bound probes to form a complementary strand
(iii) Where the probes are not bound in a contiguous manner, optionally any gaps between bound probes are filled in by polymerisation primed by said bound probes
(iv) determining the position of labels along the polymer.

In one embodiment the following steps are taken before step (i) and in another embodiment the following steps are taken before step (iv):
(i) immobilising the target molecule to a solid phase at one or more points
(ii) straightening the target molecule during or after immobilisation.

Preferably the probe of each variety is differentially labeled.

In one embodiment the complete set of oligonucleotides of a given length are provided as probes.

In one embodiment different sub-sets of probes are provided in different experiments but the probes within each sub-set are differentially labeled.

To aid detection of the location of signal along the molecule, molecules may be stretched out by methods known in the art.

5. Genomics

The invention provides a method for characterizing the physical properties or interactions of polynuleoitdes on a surface, particularly polynucleotides which are linearised on a surface.

Properties which can be determined include the chemical status, such as the state of methylation or state of depurination; and intermolecular interactions, such as the interaction of DNA regulatory regions with transcription factors.

In a preferred aspect, the invention provides a method where the nucleic acid sample is composed of DNA fibres, isolated from a cell, the method comprising substantially retaining the binding of proteins of interest and characterizing the proteins that are bound. their position on molecules which are identified and landmarks along their length have been detected.

6 Proteomics

The invention is applicable to proteomics, including the measurement of the quantities of protein species present in a sample, characterization of their properties and of the ability to interact with various partners, including small molecules, other proteins, carbohydrates, lipids and nucleic acids, or to catalyse various reactions.

The invention is particularly applicable to the analysis of the properties of protein variants created by DNA shuffling. The array may be an array as described above. In one embodiment the array is an array of nucleic acids to which a protein is linked.

In another embodiment the array is an ordered array in which each different protein is present in a different element of the array. In a further embodiment the array is a random array. In a further embodiment the array is composed of molecules isolated from a particular target organism, tissue or cell. The sample is interrogated using the following steps;
(i) producing a molecular array by a method comprising immobilising to a solid phase a plurality of molecules present in a sample, wherein the plurality of molecules are immobilised at a density such that individual molecules in the sample can be individually resolved; and
(ii) identifying and/or characterising one or more molecule immobilised to the array.

Preferably, challenging the array with sample molecule(s) of interest and detecting the interaction, where each interaction is individually resolvable. The molecules of interest are advantageously proteins, small molecules, RNA or DNA.

Preferably, contacting or approaching the array with one or more probes of interest, where each interaction is individually resolvable. Preferably, the probe is a an AFM tip and where the AFM tip is coated with a molecule or material of interest. The AFM tip can be electrically biased. Advantageously, following approach or contact by the probe the forces acting upon the probe are measured. Such forces are, for example, electrostatic forces.

Preferably, stimulating the sample with a physical agent, where each interaction is individually resolvable. Advantageously the physical agent may be electromagnetic radiation, electron source, electrical stimulation, electrochemical stimulation etc. Following stimulation with physical agent, a raman signal can be detected. Preferably, the sample is placed on metallic surfaces, preferably colloidal metal particles and surface enhanced raman signal is detected.

Advantageously, the plurality of molecules is a polypeptide repertoire or the proteome.

One or more of said immobilised molecules can be interrogated by an optical method. Preferably, the optical method is selected from far-field optical methods, near-field optical methods, epi-fluorescence spectroscopy, scanning confocal microscopy, two-photon microscopy and total internal reflection microscopy. One or more of said immobilised molecules can be interrogated by scanning probe microscopy or electron microscopy. Preferably, a physicochemical property of the immobilised molecules is determined, such as shape, size or mass, charge, hydrophobicity. Alternatively, or additionally, an electromagnetic, electrical, optoelectronic and/or electrochemical property of the immobilised molecules is determined.

In a further embodiment, a characteristic of a complex of between an immobilised molecule and a probe is determined. Preferably, the characteristics of individual immobilised molecules are learnt using a computational method. The computational method can be a neural network or artificial intelligence method such as fuzzy logic.

The invention further provides an array wherein the characteristics of a plurality of immobilised molecules and their corresponding physical location in the array have been determined. Such an array can be used in a method of identifying candidate molecules or distinguishing them from non-candidate molecules.

7 Sample Pooling

The present invention is particularly applicable to pooling strategies, such as DNA pooling in SNP typing. Pooling strategies involve mixing multiple samples together and analysing them together to save costs and time. The present invention is also applicable to detection of low frequency mutations in a wild type background. The present invention is applicable for determining haplotype frequencies in pooled DNA samples.

Labelling and Tagging Schemes for use in Single Molecule Regimes

In one embodiment the labelling schemes involve labelling with single fluorophores or a combination of single fluorophores.

In another embodiment the labelling scheme involves labelling with nanoparticles. Gold nanoparticles which are optically active and electronically active and can be made 1.4 mn in diameter (Nanoprobes) and are available derivatised with streptavidin and/or a number of fluorescent groups.

Tags can be linked to probes, such as oligonucleotide probes, in a number of ways. Firstly, probes and tags can be prepared separately and then manually linked together (not combinatorially). Secondly they can be joined by combinatorial chemistry by various means, for example, where both probe and tag are co-synthesised. Split and mix synthesis is particularly appropriate.

The present invention also provides a method for identifying and/or characterising one or more molecules of a plurality of molecules present in an array, comprising:
(i) producing a molecular array wherein the plurality of molecules are immobilised at a density such that individual molecules in the sample can be individually resolved; and
(ii) identifying and/or characterising one or more molecules immobilised in the array by contacting them with encoded tagged probes

Furthermore, the concept of using encoded probes to characterise an array may be applied to random arrays comprising immobilised molecules of interest from a sample.

The detectable feature may be present in the tag. Alternatively, the detectable feature may not be present on the tag but would be present on a partner molecule which would specifically recognize the tag. The tag and its partner can be complementary oligonucleotides or an antigen-antibody pair or a ligand-aptamer pair. The advantage of such arrangements is that bulky detectable moieties do not interfere with processing of the target molecule and is only be added once processing is completed.

Preferably each probe is encoded by virtue of being labelled with a tag which indicates uniquely the identity of the probe, such that an immobilised molecule can be identified uniquely by detecting the probes bound to the molecule and determining the identity of the corresponding tags. Consequently step (ii) may comprise contacting the immobilised molecules with a plurality of encoded probes.

In one or more of the above methods which use tagged probes and relate to the identification of nucleic acids, one or more of the tagged probes may be used to identify an individual nucleic acid molecule. For example two distal tagged probes can be used that define an area flanking one or more nucleotide sites of interest such as SNPs. Repertoires of tagged probes may also be used in methods of sequencing as described herein.

The tag repertoires may specifically be detectable by single molecule detection regimes but may also be useful in assays not requiring single molecule detection

Advantageously, the plurality of probes is labeled with a tag which indicates uniquely the identity of the probe.

Preferably, a method according to the invention is applied in single molecule detection regimes in which the number of unique tags required is reduced by using more than one tag for encoding the probe. In a preferred embodiment a unique tag is provided for each base, at each position along the sequence. Hence 24 tags species will be sufficient to code for a complete library of 6-mers. In a second preferred embodiment a unique tag is provided for each position, its quantity or some measurable feature of it is varied to encode each of the four bases. In a preferred method according to above, the tags are detectable by optical means. Further, the invention provides a method wherein the tags are particulate and comprise surface groups; a method wherein the tags are particulate and encase detectable entities, such as particle or molecules; and a method wherein tags can be detected and distinguished by scanning probe microscopy.

A invention also provides a method for tagging whereby a dendrimer is co-synthesised with the oligonucleotide sequence, where each layer of the dendrimer encodes a different base which is co-synthesized. The method also provides tags comprising nanoparticles carrying different surface or internal detectable groups that can be quantitatively detected.

The invention also provides tags that are composed of a string of beads, for example gold nanoparticles. The invention also provides tags that are composed of polymers of various lengths, the length of the polymer and optionally some other feature distinguishing one tag from another. The tags and DNA may be metallized. Analysis is by SPM or electron microscopy.

8. Biosensor

The present invention further provides a biosensor or chemosensor comprising a molecular array as defined above. The present invention also provides an integrated sensor comprising a molecular array as defined above, an excitation source, a detector, such as a CCD or alternatively integrated biosensor comprising a molecular as defined above, a voltage source and electrodes and electronic circuitry for detection. In addition, optionally means for any or all of the following: hardware-based signal processing, software-based signal processing; software-based processing of results, display of results; transmission of results to a central database on a remote computer. The present invention is particularly applicable to biosensor applications where the amount of sample material is small.

A biosensor according to the invention comprises a biosensor wherein the molecular array is formed on an optical fibre.

Moreover, the biosensor can comprise a plurality of elements, each element containing distinct molecules, such as probe sequences.

In a particular embodiment, the invention provides a biosensor for haplotyping in which:
(a) the immobilised single molecule is selectively coated with a material that facilitates detection;
(b) the coating is a conducting material which allows a circuit to form between only those electrodes onto which are occupied by the target molecule by virtue of its binding to the allelic probe present on the electrode;
(c) a potential difference is applied between electrodes in any two contiguous groups of electrodes and the electrodes on which probes interact with target are identified by virtue of the fact that a current flows between them;
(d) the conducting material comprises silver, gold, palladium or conjugated polymers; or
(e) multiple single molecules span the electrodes then the haplotype frequency is given by the amount of current that flows between the electrodes.

Advantageously, each element of the biosensor is specific for the detection of a different target, such as different pathogenic organisms. Preferably, molecules within each microarray spot are monitored.

### DESCRIPTION OF THE FIGURES

FIG. 1

Hybridisation of a single DNA Molecule to an array of pads containing allele specific probes. Labelling with a dye such as Sybr Gold enables the path of the polymer to be assessed and compared with the known position of the array pads. A. Illustrates the binding pattern of a first single molecule of a particular haplotype. B. Illustrates the binding pattern of a second single molecule of a different haplotype. C. Illustrates the capture of a single molecule by hybridization to capture probes situated on the pads. D. Illustrates signal obtained from pads where hybridisation occurs. E. Determination of alleles along a haplotype by a spatially addressable array. The signal is detected by measuring conductivity between adjacent electrodes, pairwise. If electrical continuity is detected it indicates that a DNA molecule bridges between the tested two electrodes. The DNA molecule adds as a nucleation point of a metallization process. There is little not specific metal aggregation.

FIG. 2.

Illustrates interaction of differentially tagged probes with a DNA molecule.

FIG. 3.

Illustrates the ligation of oligonucleotides bearing differentially tagged probes, templated by the DNA strand

FIG. 4.

Illustrates the binding of differentially tagged probes at distal sites from one another. Each probe then acts as a primer in a polymerisation reaction, for example using Klenow Fragment(NEB) or Taq Polymerase. The polymerisation from a first primer continues until the phosphorylated 5′ end of a second oligonucleotide is reached. At this point a DNA ligase such as E. coli DNA ligase (NEB) or Tth DNA ligase (Abgene) may ligate the extended strand to the second oligonucleotide.

FIG. 5.

Illustrates the binding of a nucleotide/oligonucleotide which is adapted with a tag. This tag is then specifically bound by a second partner molecule. The partners may be complementary oligonucleotides, antibody-antigen, streptavidin-biotin or any ligand-receptor interaction. The partners uniquely identify the probe in the context of the reaction. This is useful for example when addition of a bulky detectable label is to be avoided during the course of a reaction but can be added once the reaction has taken place.

FIG. 6.

A biosensor device is illustrated. A. A molecular beacon is shown to emit fluorescence after binding to a target molecule. This is situated on a surface structure/composition in which a waveguide is created in order to excite the dye on the beacon. Below the transparent waveguide layer is a filter and a CCD detector which detects the fluorescence emission from the opened up molecular beacon. B. An alternative molecular structure is illustrated in which a DNA intercalator acts as a FRET partner with a label on the probe. The intercalating is attached via a linker of sufficient length that the dye only comes into FRET range with it's partner when it has intercalated into a double stranded region created when the positive outcome of the assay, stable hybridisation under the defined conditions, occurs. C. it shown that each molecule within each element of the array is individually resolvable.

FIG. 7

Haplotyping on an array, The first allele is defined by the array position. The second allele is defined by the label. Each consecutive set of array spots analyses consecutive SNPs in a haplotype. The signal may be detected as a point source of fluorescence.

FIG. 8.

A. and B. Microarray scanner images of single molecule dilution series. Each DNA oligonucleotide is labelled with a single dye molecule. A and B are different exposures, C. TIRF image if a spot dilution where a few single molecules are resolvable. D, An intensity profile of a few pixels covering a putative single molecule show a one step photobelaching which is indicative of a single fluorescent dye molecule.

FIG. 9.

Oligonucleotide target labelled with a 20 nM Fluosphere nanoparticle (Molecular Probes) hybridised to complementary molecules within a spot of a single molecule array. Individual nanoparticles are easily detectable distinguishable and therefore can easily be counted. Imaging was with 40× dry Olympus (Japan) objective focused directly in the surface of microscope slide with no coverslip. The image was taken with a Roper Micromax CCD camera. The binding is specific because no binding occurred to other spots of the array

FIG. 10
Mismatch Discrimination on Single Molecule Arrays. TIRF microscopy on an Olympus Microscope. Images taken from a twofold dilution series of mismatch and perfect match probes within microarray spots that are hybridised with a complementary Cy3 labelled oligo.
It is clear that the density of hybridisation is lower in the mismatch probe spot compared to the perfect match spot at dilution 5.
The molecules are far enough apart to enable single molecules to be easily counted at dilution 5 for the mismatch whereas they only become far enough apart at dilutions 7/8 for the perfect match. Gamma setting 0 to 2000 for all.

FIG. 11.

Single Molecule Counting, The microarrays spot image is digitised so that individual molecules can be assessed The number of molecules in dilution 5 are seen to be less than in dilution 4. Objects which the software deems as individual molecules are coloured so that they can easily visualized. Non-single molecule objects are in white.

FIG. 12.

Illustration of the capture-combing process within a microarray spot. A. Capture probes on a surface (primary array). B. Capture of sample from solution. C. Combing of captured nucleic acids (secondary array)

FIG. 13

Self-assembly and horizontalization of genomic DNA on DNA microarrays.
Illustration of the concept of sorting and displaying the genome by sequence specific capture of different fragments of the genome to different locations on an array surface.

FIG. 14

Images of the experimental results of capture combing genomic DNA to a spatially addressable array at various magnifications. Straightened, individually resolvable single DNA polymers clearly seen in the 100× magnification images (A and C) the DNA polymers are seen only within the spot areas. There is no combing in areas outside the spots, where the capture probes are not present

FIG. 15.

A. Concatemerised Lambda DNA of >200 kb length B. Concatemerised genomic DNA, probed at a recurring sequence in the concatamers

FIG. 16.

Spread of Human Female Genomic DNA on a Matsunami coverslip. 100× Olympus objective.

FIG. 17.

An array of electrodes(dark areas) separated by gaps. Adjacent electrodes are spotted with oligonucleotides complementary to different ends of a lambda DNA molecule. At the top right hand corner a single Lambda molecule can be seen which is bridging the two electrodes by binding to its complementary oligonucleotide. Hybridisation was done in 4× SSC/o.1% Sarkosyl.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods. See also Genomics, The Science and Technology Behind the Human Genome Project [1999]; Charles Cantor and Cassandra Smith (John Wiley and Sons) for genomics technology and methods including sequencing by hybridisation. DNA Microarray: A Practical Approach [1999] Ed: M. Schena, (Oxford University Press) can be referred to for array methods.

The present invention possesses many advantages over conventional bulk analysis of molecular arrays. One of the key advantages is that, in accordance with the present invention, specific PCR amplification of target molecules may be dispensed with due to the sensitivity of single molecule analysis. Thus, there is no requirement to amplify target nucleic acids, which is a very cumbersome task when analysis is large scale or requires rapid turnaround and which may introduce errors due to non-linear amplification of target strands and the under-representation of rare molecular species often encountered with PCR. It also adds considerable expense.

Moreover, the methods of the invention may be multiplexed to a very high degree. Samples may comprise pooled genomes of target and control subject populations respectively, since accurate analysis of allele frequencies may be accurately determined by single molecule counting. Since more than a single site on each molecule may be probed, haplotype information is easily determined. There is also the possibility of obtaining haplotype frequencies. Such methods are particularly applicable in association studies, where SNP frequencies are correlated with diseases in a population. The expense of single SNP typing reactions can be prohibitive when each study requires the performance of millions of individual reactions; the present invention permits millions of individual reactions to be performed and analysed on a single array surface.

A. Methods of Manufacturing Low Density Arrays.

The present invention is in one aspect concerned with the production of molecular arrays wherein the individual molecules in the array are at a sufficiently low density such that the individual molecules can be individually resolved—i.e. when visualised using the method of choice, each molecule can be visualised separately from neighbouring molecules, regardless of the identity of those neighbouring molecules. The required density varies depending on the resolution of the visualisation method. As a guide, molecules are preferably separated by a distance of approximately at least 250, 500, 600, 700 or 800 nm in both dimensions when the arrays are intended for use in relatively low resolution optical detection systems (the diffraction limit for visible light is about 300 to 500 nm). If nearest neighbour single molecules are labelled with different fluors, or their functionalization can be temporally resolved, then it may be possible to obtain higher resolution by deconvolution algorithms/image processing. Alternatively, where higher resolution detection systems are used, such as scanning near-field optical microscopy, then separation distances of 50 nm or more may be used. As detection techniques improve, it may be possible to reduce further the minimum distance. The use of non-optical methods, such as AFM, allows the reduction of the feature-to-feature distance effectively to zero.

Since, for example, during many immobilisation procedures or density reduction procedures, the probability of all molecules being at least the minimum distance required for resolution is low, it is acceptable for a proportion of molecules to be closer than that minimum distance. However, it is preferred that at least 50%, more preferably at least 75, 90 or 95% are at the minimum separation distance required for individual resolution.

Furthermore, the actual density of molecules in the array may be higher than the maximum density allowed for individual resolution since only a proportion of those molecules may be detectable using the resolution method of choice. Thus where resolution, for example, involves the use of labels, then provided that individually labelled molecules can be resolved, the presence of higher densities of unlabeled molecules is immaterial. The label may be due to the sample molecules which may be low in number compared to the probe molecules.

Molecules that may be immobilised to the array include nucleic acids such as DNA and analogues and derivatives thereof, such as PNA. Nucleic acids may be obtained from any source, for example genomic DNA or cDNA or synthesised using known techniques such as step-wise synthesis. Nucleic acids may be single or double stranded. DNA nanostructures or other supramolecular structures may also be immobilised. Other molecules include: compounds joined by amide linkages such as peptides, oligopeptides, polypeptides, proteins or complexes containing the same; defined chemical entities, such as organic molecules; combinatorial libraries; conjugated polymers and carbohydrates.

In several embodiments, the chemical identity of the molecules must be known or encoded prior to manufacture of the array by the methods of the present invention. For example, the sequence of nucleic acids (or at least the sequence of the region that is used to bind sample molecules) and the composition and structure of other compounds should be known or encoded in such a way that the sequence of molecules of interest can be determined with reference to a look-up table. The term “spatially addressable, as used herein, therefore signifies that the location of a molecule specifies its identity (and in spatial combinatorial. synthesis, the identity is a consequence of location).

However, in alternative embodiments, arrays may be manufactured using pluralities of unknown molecules from samples and the arrays subsequently interrogated to characterise and identify the immobilised molecules, particularly by using encoded probes. The characteristics and location of individual immobilised molecules may then be determined using encoded probes and the results “learnt” for future use. Learning may be achieved using computational methods such as neural networks or artificial intelligence.

Molecules may be labelled to enable interrogation using various methods. Suitable labels include: optically active dyes, such as fluorescent dyes; nanoparticles such as fluorospheres and quantum dots; and surface plasmon resonant particles (PRPs) or resonance light scattering particles (RLSs)—particles of silver or gold that scatter light (the size and shape of PRP/RLS particles determines the wavelength of scattered light). (Schultz et al., 2000, PNAS 97: 996-1001; Yguerabide, J. and Yguerabide E., 1998, Anal Biochem 262: 137-156). Quantum dots or rods or nanobars can also be used.

In the resulting arrays, it is preferred that molecules are arranged in discrete elements. Generally, each element is adjacent to another or at least 1 μm apart and/or less than 10, 20, 50, 100 or 300 μm apart. Each element is spatially addressable since the identity of the molecules present in each element is known or can be determined on the basis of a prior coding. Thus if an element is interrogated to determine whether a given molecular event has taken place, the identity of the immobilised molecule is already known by virtue of its position in the array. Within each element, only one molecule species may be present, in single or multiple copies. Where present in multiple copies, it is preferred that individual molecules are individually resolvable. In one embodiment, elements in the array may comprise multiple species that are individually resolvable. Typically, multiple species are differentially labelled such that they can be individually distinguished. By way of an example, an element may comprise a number of different probes for detecting single nucleotide polymorphisms alleles, each probe having a different label such as a different fluorescent dye.

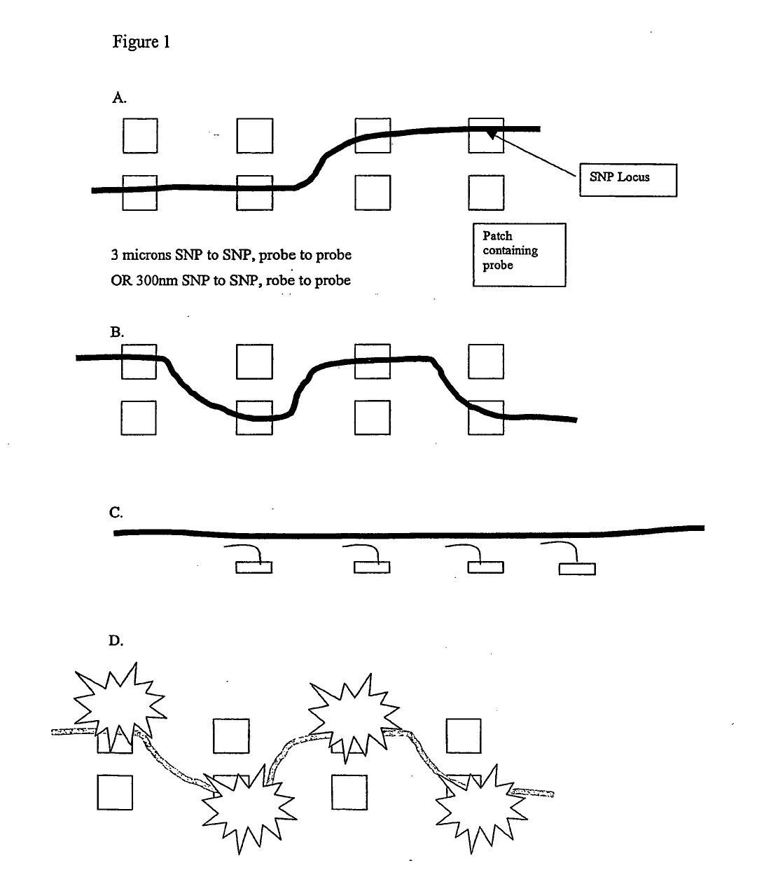

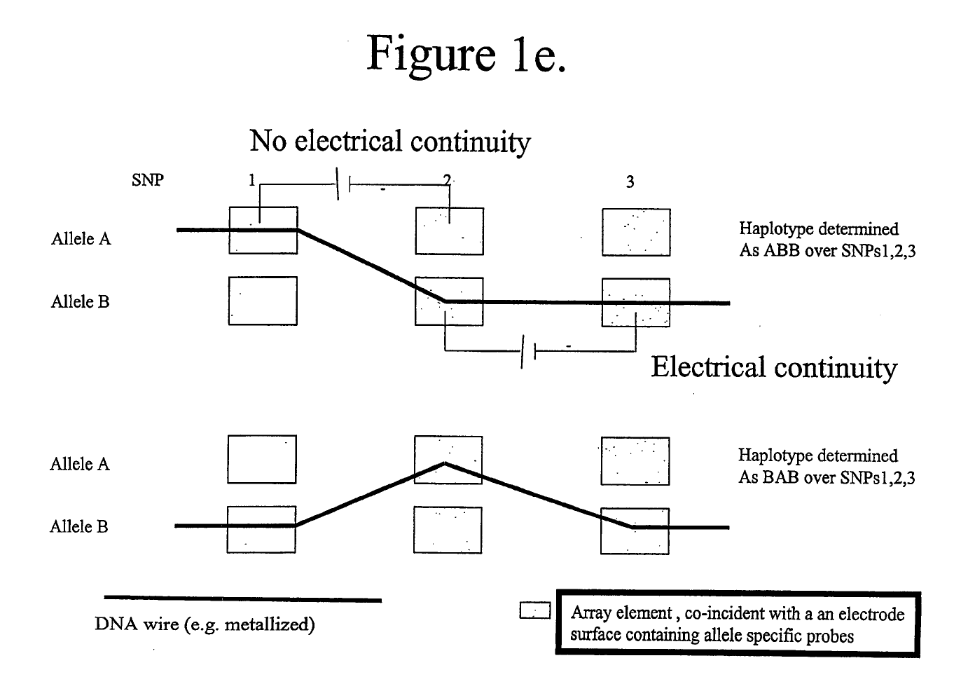

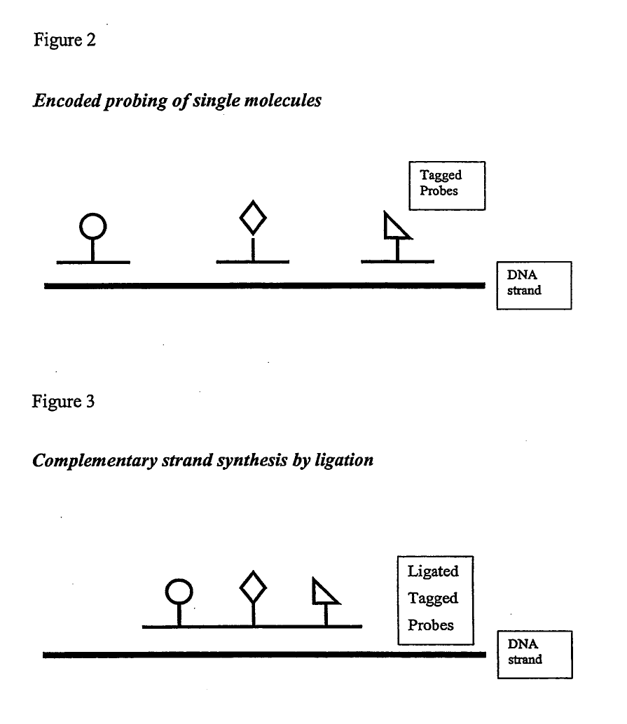

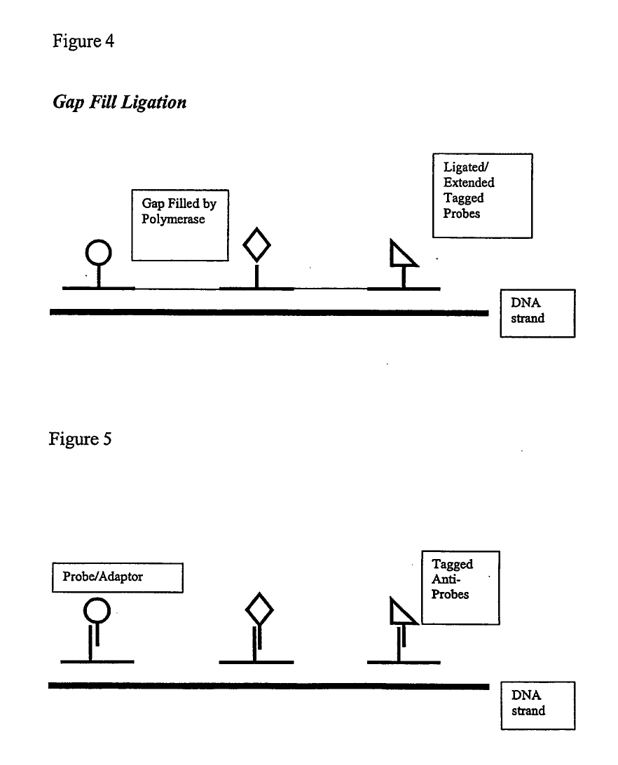

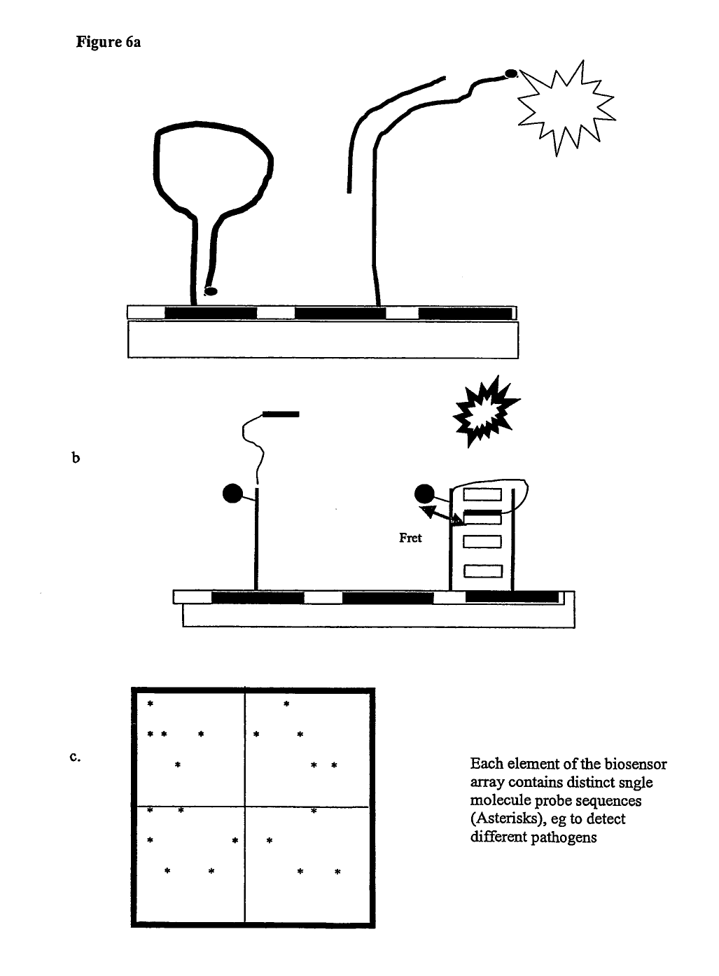

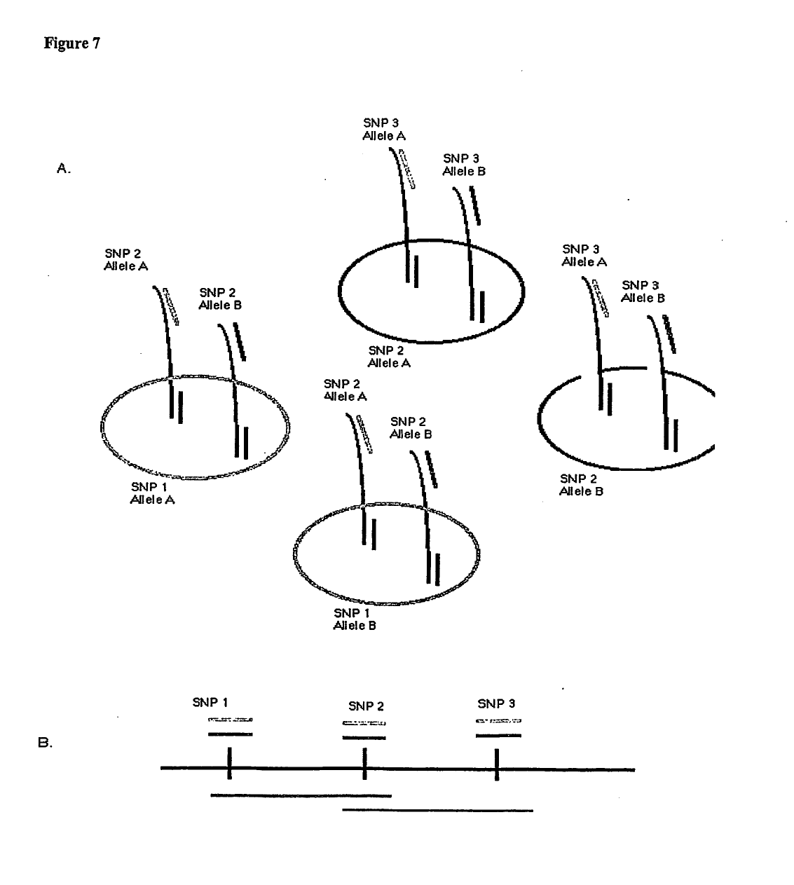

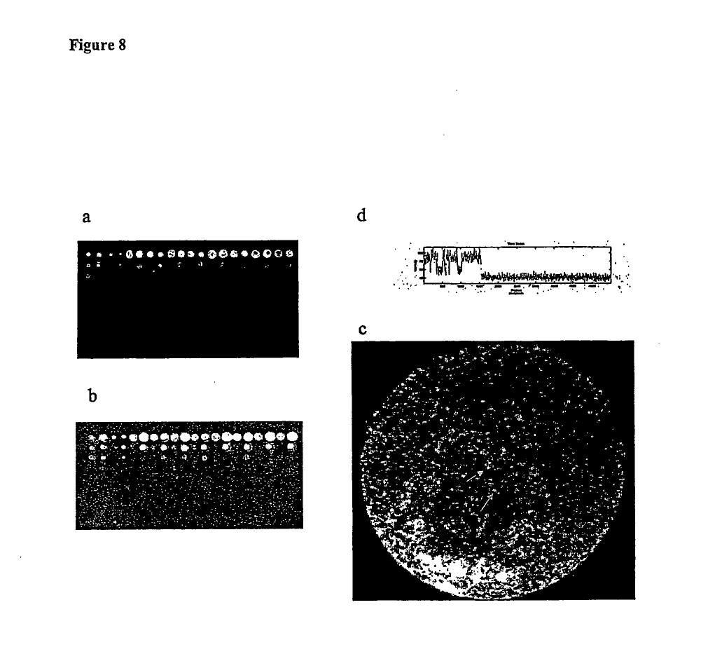

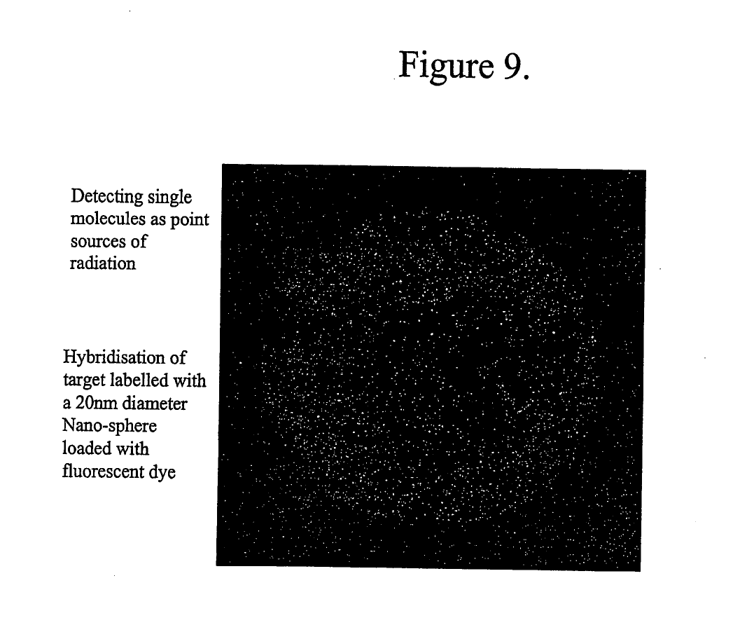

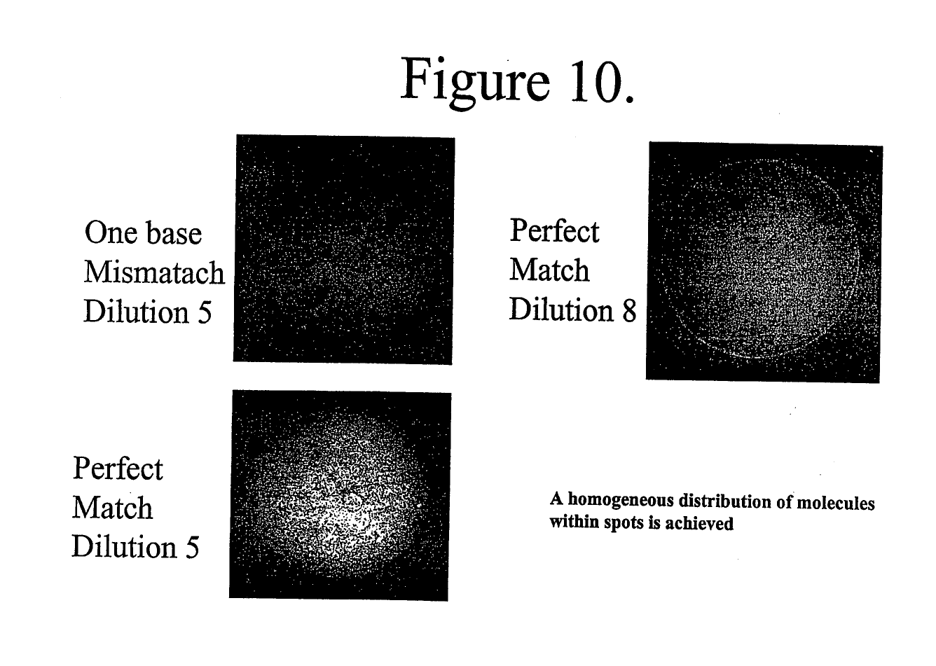

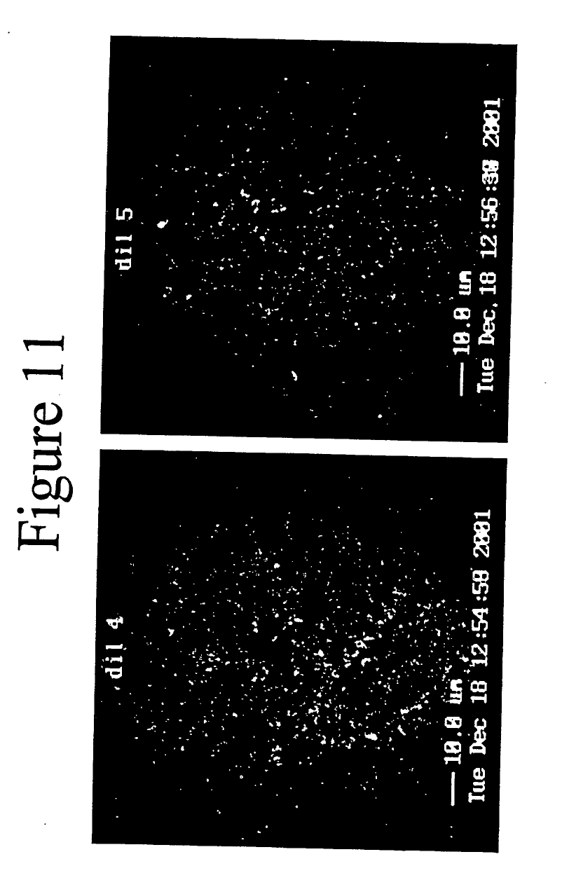

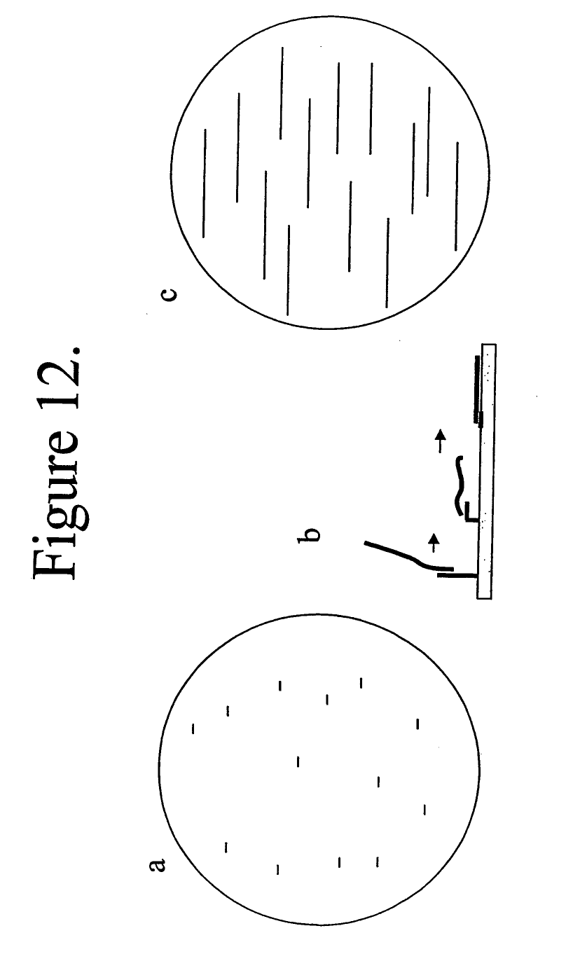

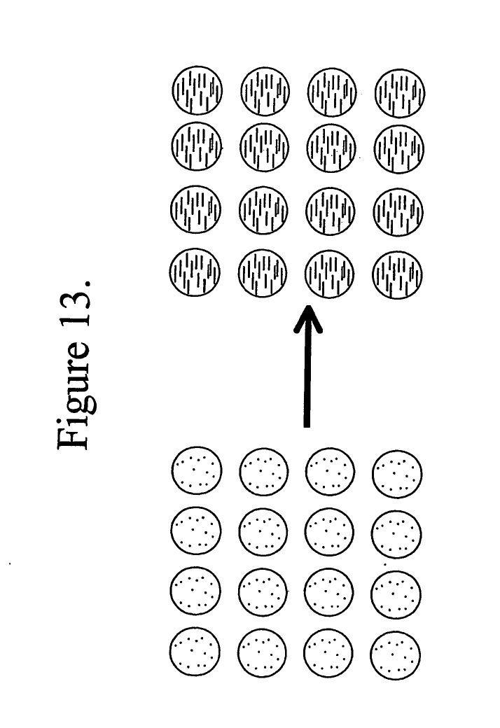

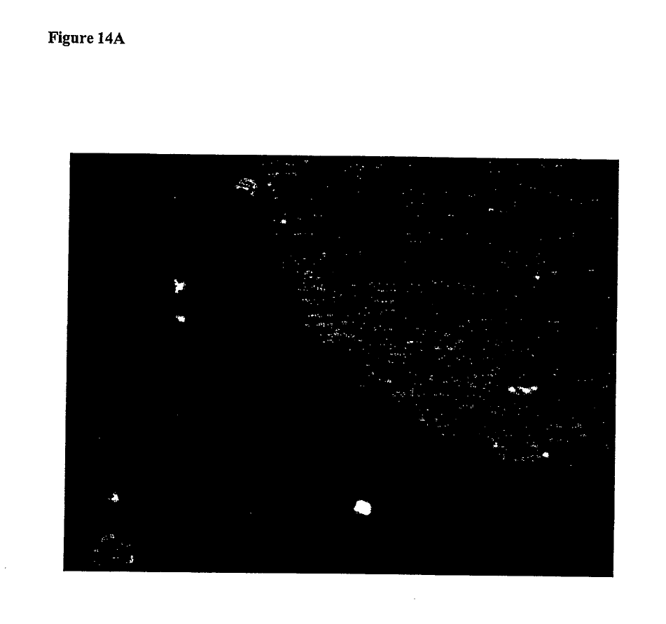

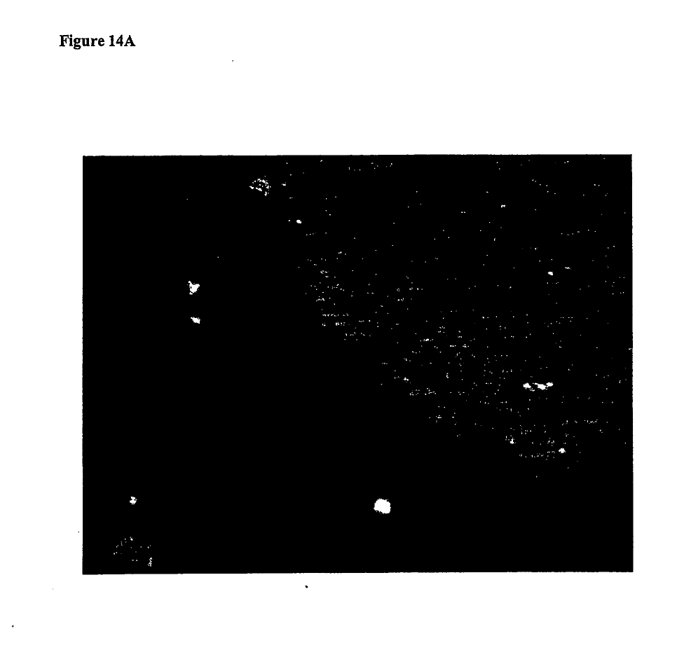

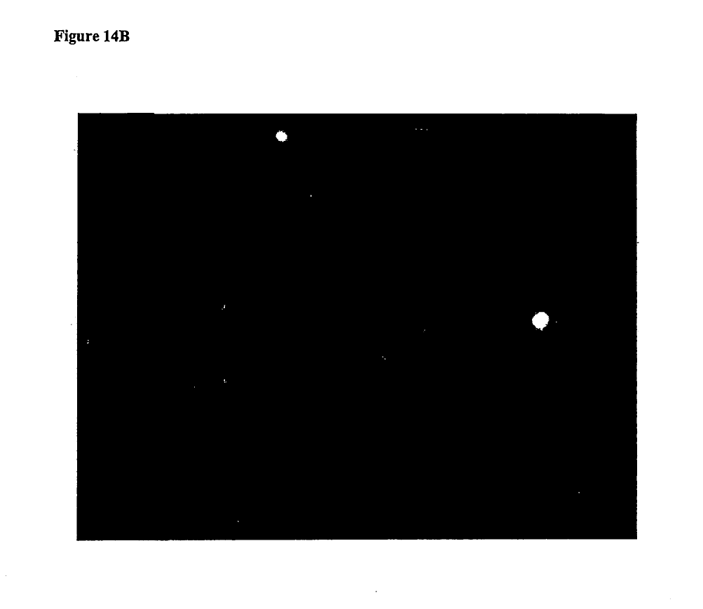

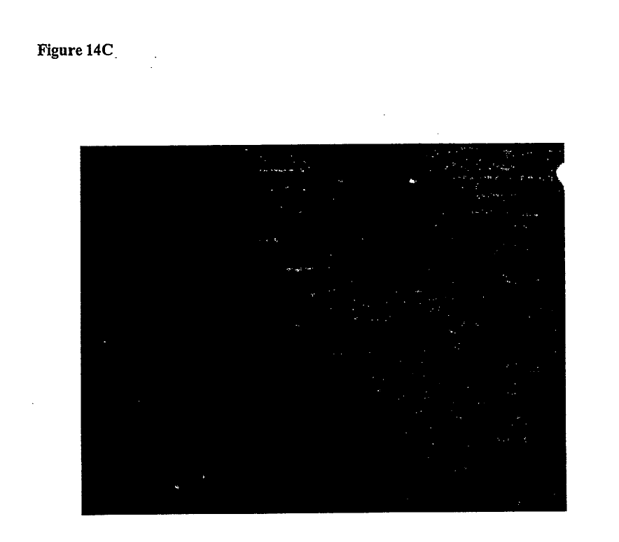

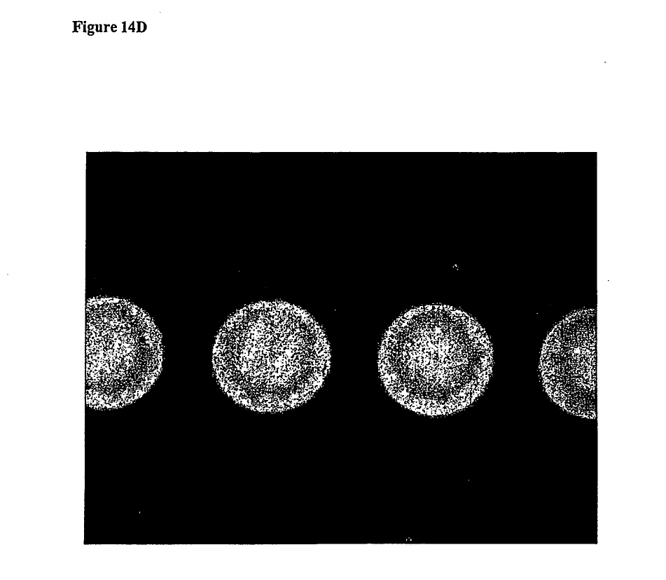

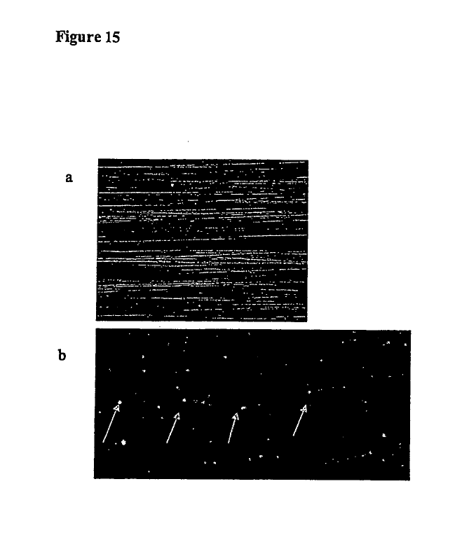

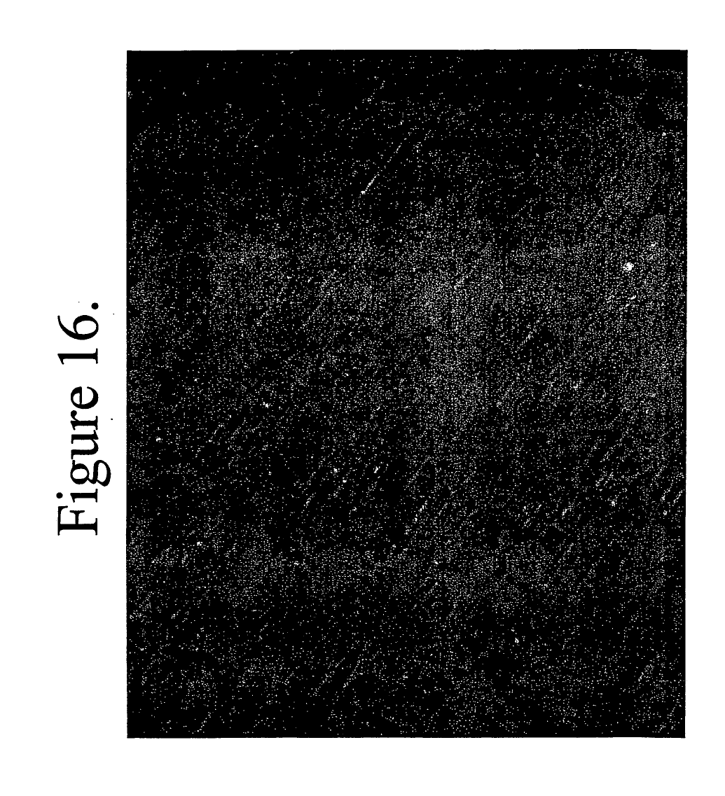

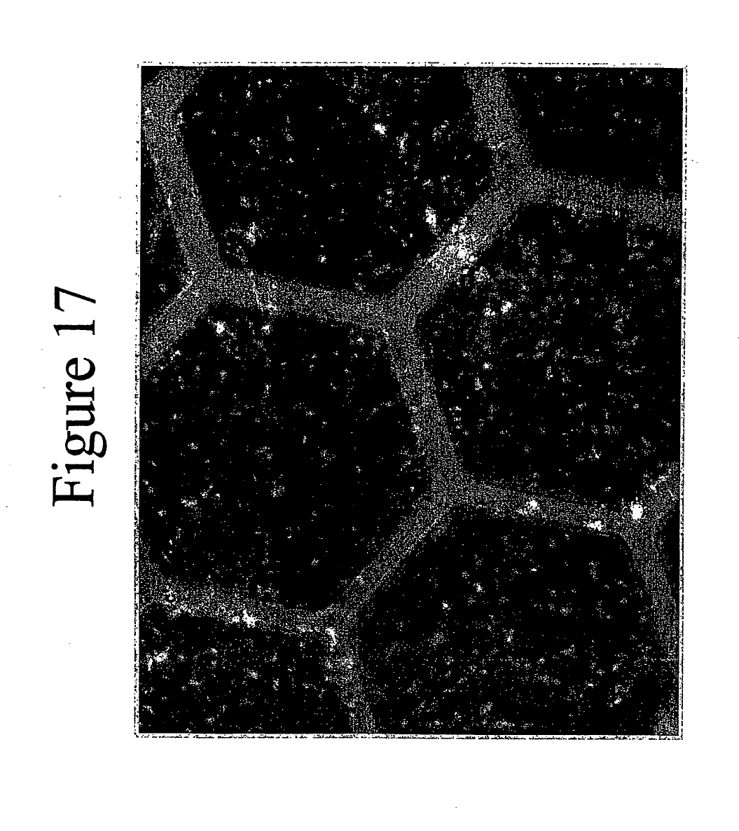

In one embodiment, the array comprises a block of array elements where probes specific for different SNP alleles are grouped together, typically in separate but adjacent discrete elements. Furthermore, groups of probes which detect different but closely linked SNP loci may be arranged together in the block of array elements. In this way, a block of elements may be used to probe multiple loci in a single molecule simultaneously. The distance between the probes for different loci will generally be determined by the distance between the loci in the target nucleic acid molecules. For example, if the SNP loci are 10 kb apart, then each group of allelic probes may be spaced apart by about 3 microns. If the SNPs are about 1000 bp apart then each group of allelic probes may be spaced apart by about 300 nm. In practice the distance between each consecutive SNP would vary. In a highly preferred embodiment, the various probes in the block of array elements are arranged such that the groups of allelic probes for the various loci are arranged in one axis and within each group, the different allelic probes for the locus are arranged in another axis. For example, to detect four linked biallelic SNP loci, a block of array elements may be arranged as 8 cells in a 4 by 2 arrangement with the probes for one allele on one row and the probes for the other allele on another row, each column having two cells representing the two possible alleles for each locus (see FIG. 1).

This arrangement of blocks of array elements for interrogating individual molecules at multiple loci is not limited to SNP detection but may also be used in other methods such as haplotyping or sequence determination.

Molecular arrays produced by the methods of the invention preferably comprise at least 10 distinct molecular species, more preferably at least 50 or 100 different molecular species.

Two possible approaches for manufacturing low density arrays for use in the present invention are outlined below.

i. De Novo Fabrication

In one embodiment of the present invention, low density molecular arrays are produced by immobilising pluralities of molecules of known composition to a solid phase. Typically, the molecules are immobilised onto or in discrete regions of a solid substrate. The substrate may be porous to allow immobilisation within the substrate (e.g. Benoit et al., 2001, Anal. Chemistry 73: 2412-242) or substantially non-porous, in which case the molecules are typically immobilised on the surface of the substrate.

The solid substrate may be made of any material to which the molecules can be bound, either directly or indirectly. Examples of suitable solid substrates include flat glass, quartz, silicon wafers, mica, ceramics and organic polymers such as plastics, including polystyrene and polymethacrylate. The surface may be configured to act as an electrode or a thermally conductive substrate (which may enhance the hybridisation or discrimination process). For example, micro and sub-micro electrodes can be formed on the surface of a suitable substrate using lithographic techniques. Smaller nanoelectrodes can be made by electron beam writing/lithography. Electrodes may also be made using conducting polymers which may be applied to the substrate by ink-jet printing devices or by soft lithography. Electrodes may be provided at a density such that each immobilised molecule has its own electrode or at a higher density such that groups of molecules or elements are connected to an individual electrode. Alternatively, one electrode may be provided as a layer below the surface of the array which forms a single electrode. Where each probe species are arranged on individual electrodes, the current flowing between separate electrodes can be determined. A current would be expected to flow if certain molecules, such as double stranded DNA or conductive substances whose growth is templated by such molecules span the space between the electrodes.

The solid substrate may optionally be interfaced with a permeation layer or a buffer layer. It may also be possible to use semi-permeable membranes such as nitrocellulose or nylon membranes, which are widely available. The semi-permeable membranes may be mounted on a more robust solid surface such as glass. The surfaces may optionally be coated with a layer of metal, such as gold, platinum or other transition metal. A particular example of a suitable solid substrate is the commercially available SPR BIACore™ chip (Pharmacia Biosensors). Heaton et al., 2001 (PNAS 98:3701-3704) have applied an electrostatic field to an SPR surface and used the electric field to control hybridisation.

Preferably, the solid substrate is generally a material having a rigid or semi-rigid surface. In preferred embodiments, at least one surface of the substrate will be substantially flat, although in some embodiments it may be desirable to physically separate discrete elements with, for example, raised regions or etched trenches. For example, the solid substrate may comprise nanovials—small cavities in a flat surface e.g. 10 μm in diameter and 10 μm deep. This may particularly be useful for cleaving molecules from a surface and performing assays or other processes such as amplification on them. The solution phase reaction would be expected to be more efficient than the solid phase reaction. But the result would remain spatially addressable which is advantageous.

It is also preferred that the solid substrate is suitable for the low density application of molecules such as nucleic acids in discrete areas. It may also be advantageous to provide channels to allow for capillary action since in certain embodiments this may be used to achieve the desired straightening of individual nucleic acid molecules. Channels may be in a 2-D arrangement (e.g Quake S, and Scherer., 200, Science 290: 1536-1540) or in a 3-D flow through arrangement (Benoit et al., 2001, Anal.Chemistry 73: 2412-2420). Channels could provide a higher surface area hence a larger number of molecules could be immobilised. In the case of a 3-D flow channel array interrogation may be by confocal microscopy which may image multiple slices of the channels in the z direction.

In some instances array elements will be raised atop electrodes/electrode arrays.

The solid substrate is conveniently divided up into sections. This may be achieved by techniques such as photoetching, or by the application of hydrophobic inks, for example Teflon-based inks (Cel-line, USA).

Discrete positions, in which each different molecules or groups of molecular species are located may have any convenient shape, e.g., circular, rectangular, elliptical, wedge-shaped, etc.

Attachment of the plurality of molecules to the substrate may be by covalent or non-covalent (such as electrostatic) means. The plurality of molecules may be attached to the substrate via a layer of intermediate molecules to which the plurality of molecules bind. For example, the plurality of molecules may be labelled with biotin and the substrate coated with avidin and/or streptavidin. A convenient feature of using biotinylated molecules is that the efficiency of coupling to the solid substrate can be determined easily. Since the plurality of molecules may bind only poorly to some solid substrates, it may be necessary to provide a chemical interface between the solid substrate (such as in the case of glass) and the plurality of molecules. Examples of suitable chemical interfaces include various silane linkers and polyethylene glycol spacer. Another example is the use of polylysine coated glass, the polylysine then being chemically modified if necessary using standard procedures to introduce an affinity ligand. Nucleic acids may be immobilised directly to a polylysine surface (electrostatically). The surface density of the surface charge will be important to immobilise molecules in a manner that allows them to be well. presented for assays and detection.

Other methods for attaching molecules to the surfaces of solid substrate by the use of coupling agents are known in the art, see for example WO98/49557. The molecules may also be attached to the surface by a cleavable linker.

In one embodiment, molecules are applied to the solid substrate by spotting (such as by the use of robotic micropipetting techniques—Schena et al., 1995, Science 270: 467-470) or ink jet printing using for example robotic devices equipped with either pins or piezo electric devices as in the known art.

For example pre-synthesised oligonucleotides dissolved 100 mM NaoH or 2-4×SSC can be applied to glass slides coated with 3-Glycodioxypropyltrimethoxysilane or the ethoxy derivative under con. These can then be placed at 110 degrees for 15 minutes and then placed at 4 degrees. Advantageously the oligos may have an amino terminus but unmodified oligos can also be spotted.

Alternatively amino-terminated oligonucleotides can be spotted onto 3-Aminopropyltrimethoxysilane in 100 mM 1:1 Sodium Carbonate: Sodium Hydrogen Carbonate at pH 9. This can be followed by 37 degrees for two hours and exposure to ammonia vapour for 1 hour.

CDNAs or other unmodified DNA can be spotted onto the above slides or onto poly-L-lysine coated slides can 2-4×SSC or 1:1 DMSO: Water can be used for spotting. Optional treatment with UV and succinic anhydride. There are a number of vendors who sell slides with different surface modifications and appropriate buffers e.g Corning (USA), Qunatifoil (Jena. Germany), Surrmodics (USA),Mosaic (Boston, USA).

The required low density is typically achieved by using dilute solutions. One microlitre of a 10⁶ M solution spread over a 1 cm² area has been shown to give a mean intermolecular separation of 12.9 nm on the surface, a distance far too small to resolve with optical microscope. Each factor of 10 dilution increases the average intermolecular separation by a factor 3.16. Thus, a 10⁻⁹ M solution gives a mean intermolecular separation of about 400 nm and a 10⁻¹² M gives a mean intermolecular separation of about 12.9 μm. With a mean separation of about 12.9 μm, if the molecules are focused to appear to be 0.5 μM in diameter and the average distance is 5 μM, then the chance of two molecules overlapping (i.e. centre to centre distance of 5 μM or less) is about 1% (based on M. Unger E. Kartalov, C. S Chiu, H. Lester and S. Quake, “Single Molecule Fluorescence Observed with Mercury Lamp Illumination”, Biotechniques 27: 1008-1013 (1999)). Consequently, typical concentrations of dilute solutions used to spot or print the array, where far field optical methods will be used for detection will be in the order of at least 10⁻⁹ M, preferably least 10⁻¹⁰ M or 10⁻¹² M. The concentration used will be higher with the use of superresolution far field methods or SPM. It should also be borne in mind that only a fraction of molecules that are spotted onto a surface will robustly attach to the surface (0.1% to 1% for example). Depending on the method of immobilisation, only a fraction of those molecules that are robustly attached will be available for hybridisation or enzymatic assays. For example with the use of aminolinked oligonucleotides and spotting onto a Aminopropyltriethoxysilane (APTES) coated slide surface about 20% of the oligonucleotides are available for mini-sequencing.

In a second embodiment, the surface is designed in such a way that sites of attachment (i.e. chemical linkers or surface moieties) are dilute or that sites are selectively protected or blocked. In this case, the, concentration of the sample used for ink jet printing or spotting is immaterial provided the attachment is specific to these sites. In the case of in situ synthesis of molecules, the lower number of available sites for initiating synthesis allows more efficient synthesis providing a higher chance of obtaining full-length products.

Polymers such as nucleic acids or polypeptides may also be synthesised in situ using photolithography and other masking techniques whereby molecules are synthesised in a step-wise manner with incorporation of monomers at particular positions being controlled by means of masking techniques and photolabile reactants. For example, U.S. Pat. No. 5,837,832 describes a method for producing DNA arrays immobilised to silicon substrates based on very large scale integration technology. In particular, U.S. Pat. No. 5,837,832 describes a strategy called “ling” to synthesise specific sets of probes at spatially-defined locations on a substrate. U.S. Pat. No. 5,837,832 also provides references for earlier techniques that may also be used. Light directed synthesis can also be carried out by using a Digital Light Micrornirror chip (Texas Instruments) as described (Singh-Gasson et al Nature Biotechnology 1999 17: 974-978). Instead of using photo-deprotecting groups which are directly processed by light, conventional deptotecting groups such as dimethoxy trityl can be employed with light directed methods where for example a photoacid is generated in a spatially addressable way which selectively deprotects the DNA monomers (McGall et al PNAS 1996 93: 1355-13560). Electrochemical generation of acid is another means that is being developed (eg. Combimatrix Corp.)

The size of array elements is from 0.1×0.1 microns and above as can be ink jet printed onto a patterned surface or created by photolithography or physical masking.

Molecules may be attached to the solid phase at a single point of attachment, which may be at the end of the molecule or otherwise. Alternatively, molecules may be attached at two or more points of attachment. In the case of nucleic acids, it may be advantageous to use techniques that ‘horizontalize’ the immobilised molecule relative to the solid substrate. For example, fluid fixation of drops of DNA has been shown previously to elongate and fix DNA to a derivatised surface such as silane derivatised surfaces. This may promote accessibility of the immobilised molecules for target molecules. Spotting of sample by quills/pins/pens under fast evaporation conditions creates capillary forces as samples dry to elongate molecules. Means for straightening molecules by capillary action in channels have been described by Jong-in Hahm at the Cambridge Healthtech institutes Fifth Annual meeting on Advances in Assays, Molecular Labels, Signaling and Detection, May 17-18^{th} Washington D.C. Samples may be applied through an array of channels. The density of molecules stretched across a surface is typically constrained by the radius of gyration of the DNA molecule.

Immobilised molecules may also serve to bind further molecules to complete manufacture of the array. For example, nucleic acids immobilised to the solid substrate may serve to capture further nucleic acids by hybridisation, or polypeptides. Similarly, polypeptides may be incubated with other compounds, such as other polypeptides. It may be desirable to permanently “fix” these interactions using, for example UV cross-linking and appropriate cross-linking reagents. Capture of secondary molecules may be achieved by binding to a single immobilised “capture” molecules or to two or more “capture” molecules. Where secondary molecules bind to two or more “capture” molecules, this may have the desirable effect of containing the secondary molecule horizontally.

ii. Density Reduction of High Density Arrays

In an alternative embodiment, the molecular array may be obtained by providing an array produced with molecules at normal (high) densities using a variety of methods known in the art, followed by reduction of surface coverage.

A reduction in actual or effective surface coverage may be achieved in a number of ways. Where molecules are attached to the substrate by a linker, the linker may be cleaved. Instead of taking the cleavage reaction to completion the reaction is partial, to the level required for achieving the desired density of surface coverage. In the case of molecules attached to glass by an epoxide and PEG linkage, such as oligonucleotides, partial removal of molecules can be achieved by heating in ammonia which is kinown to progressively destroy the lawn.

It may also be possible to obtain a reduction in surface coverage by functional inactivation of molecules in situ, for example using enzymes or chemical agents. The amount of enzyme or agent used should be sufficient to achieve the desired reduction without inactivating all of the molecules. Although the end result of this process will often be a substrate which has molecules per se at the same density as before the density reduction step, the density of functional molecules will have been reduced since many of the original molecules will have been inactivated. For example, phosphorylation of the 5′ ends of 3′ attached oligonucleotides by Polynucleotide kinase, which renders the oligonucleotides available for ligation assays may only be 10% efficient.

An alternative method for obtaining a reduction in molecule density is to obtain an effective reduction in density by labelling or tagging only a proportion of the pre-existing immobilised molecules so that only the labelled/tagged molecules at the required density are available for interaction and/or analysis. This is particularly useful for analysing low target numbers on normal density arrays where the target introduces label.

These density reduction steps can be applied conveniently to ready-made molecular arrays which are sold by various vendors e.g. Affymetrix, Corning, Agilent and Perkin Elner. Alternatively, proprietary molecular arrays may be treated as required.

The present invention also provides an “array of arrays”, wherein an array of molecular arrays (level 1) as described are configured into arrays (level 2) for the purpose of multiplex analysis. Multiplex analysis can be done by sealing each molecular array (level 1) by individual chambers, that makes a seal with the common substrate, so that a separate sample can be applied to each. Alternatively each molecular array (level 1) can be place at the end of a pin (as commonly used in combinatorial chemistry) or a fibre and can be dipped into a multi well plate such as a 384 well microtitre plate. The fibre could be an optical fibre which can serve to channel the signal from each array to a detector. The molecular array (level 1) could be on a bead which self-assembles onto a hollow optical fibre as described by Walt and co-workers Mumina Inc. Karri et al Anal. Chem 1998 70: 1242-1248]. Moreover, the array maybe of of arrays of randomly immobilised molecules of known and defined type, for example a complete oligonucleotide set of every 17 mer or genomic DNA from a particular human sample.

Biosensors

Low density molecular arrays may be used to produce a biosensor which may be used to monitor single molecule assays on a substrate surface, such as a chip. The array may comprise, for example, between 1 and 100 different immobilised molecules (e.g. probes), an excitation source and a detector such as a CCD, all within an integrated device. Sample processing may or may not be integrated into the device.

In one aspect, the biosensor would comprise a plurality of elements, each element containing distinct molecules, such as probe sequences. Each element may then be specific for the detection of, for example, different pathogenic organisms.

In a preferred embodiment the immobilised molecules would be in the form of molecular beacons and the substrate surface would be such that an evanescent wave can be created at the surface. This may be achieved by the forming a grating structure on the substrate surface or by making the array on an optical fibre (within which light is totally internally reflected) for example. The CCD detector may be placed below the array surface or above the array, separated from the surface by a short distance to allow space for the reaction volume.

Examples of biosensor configurations are given in FIG. 6 where: (a) is an integrated detection scheme based on Fluorescence Energy Resonance Transfer (FRET). The sample is applied between two plates, one with a CCD and the other with an LED with grating structure on its surface. (b) is an integrated detection system with a molecular beacon (Tyagi et al Nat Biotechnol. 1998, 16:49-53) on an optical fibre.

B. Interrogation/Detection Methods

Individual molecules in the array and their interaction with target molecules can be detected using a number of means. Detection may be based on measuring, for example physicochemical, electromagnetic, electrical, optoelectronic or electrochemical properties, or characteristics of the immobilised molecule and/or target molecule.

There are two factors that are pertinent to single molecule detection of molecules on a surface. The first is achieving sufficient spatial resolution to resolve individual molecules. The density of molecules is such that only one molecule is located in the diffraction limit spot of the microscope which is ca. 300 nm. Low signal intensities reduce the accuracy with which the spatial position of a single molecule can be determined. The second is to achieve specific detection of the desired single molecules as opposed to background signals.

Scanning probe microscopy (SPM) involves bringing a probe tip into intimate contact with molecules as the tip is scanned across a relatively flat surface to which the molecules are attached. Two well-known versions of this technique are scanning tunnelling microscopy (STM) and atomic force microscopy (AFM; see Moeller et al., 2000, NAR 28: 20, e91) in which the presence of the molecule manifests itself as a tunnel current or a deflection in the tip-height of the probe, respectively. AFM may be enhanced using carbon nanotubes attached to the probe tip (Wooley et al., 2000, Nature Biotechnology 18: 760-763). An array of SPM probes which can acquire images simultaneously, are being developed by many groups and this would speed the image acquisition process. Gold or other material beads could be used to help scanning probe microscopy find molecules automatically. Electron microscopy is also a means to interrogate but this is relatively cumbersome.

Optical methods based on sensitive detection of absorption or emission may also be used Typically optical excitation means are used to interrogate the array, such as light of various wavelengths, often produced by a laser source. A commonly used technique is laser-induced fluorescence. Although some molecules will be sufficiently inherently luminescent for detection, generally molecules in the array (and/or target molecules) will need to be labelled with a chromophore such as a dye or optically active particle (see above). If necessary, the signal from a single molecule assay can, for example, be amplified by labelling with dye loaded nanoparticles, or multi-labelled dendrimers or PRPs/SPRs. Raman spectroscopy is another means for achieving high sensitivity.

Plasmon resonant particles (PRPs) are metallic nanoparticles which scatter light elastically with remarkable efficiency because of a collective resonance of the conduction electrons in the metal (i.e. the surface plasmon resonance). PRPs can be formed that have scattering peak anywhere in the visible range of the spectrum. The magnitude, peak wavelength and spectral bandwidth of the plasmon resonance associated with a nanoparticle are dependent on a particle's size, shape and material composition, as well as local environment. These partcles can be used to label a molecule of interest.

SERS [Surface-enhanced Raman Scattering on nanoparticles exploit raman vibrations on metallic nanoparticles of the single molecules themselves to amplify their spectroscopic signatures.

Further, many of these techniques may be applied to fluorescence resonance energy transfer (FRET) methods of detecting interactions where, for example the molecules in the array are labelled with a fluorescent donor and the target molecules (or reporter oligonucleotides) are labelled with a fluorescent acceptor, a fluorescent signal being generated when the molecules are in close proximity. Moreover, structures such as molecular beacons where the FRET donor and acceptor (quencher) are attached to the same molecule can be used.

The use of dye molecules encounters the problems of photobleaching and blinking. Labelling with dye-loaded nanoparticles or surface plasmon resonance (SPR) particles reduces the problem. However a single dye molecule will bleach after a period of exposure to light. The photobleaching characteristics of a single dye molecule have been used to advantage in the single molecule field as a means for distinguishing signal from multiple molecules or other particles from the single molecule signal.

Spectroscopy techniques require the use of monochromatic laser light, the wavelength of which will vary according to the application. However, microscopy imaging techniques may use broader spectrum electromagnetic sources.

Optical interrogation/detection techniques include near-field scanning optical microscopy (NSOM), confocal microscopy and evanescent wave excitation. More specific versions of these techniques include far-field confocal microscopy, two-photon microscopy, wide-field epi-illumination, epifluorescence microscopy and total internal reflection (TIR) microscopy. Many of the above techniques may also be used in a spectroscopic mode. The actual detection means include charge coupled device (CCD) cameras and intensified CCDs, photodiodes and photomultiplier tubes. These means and techniques are well-known in the art. However, a brief description of a number of these techniques is provided below.

Near-Field Scanning Microscopy (NSOM)

In NSOM, subdiffraction spatial resolutions in the order of 50-100 nm are achieved by bringing a sample to within 5-10 nm of a subwavelength-sized optical aperture. The optical signals are detected in the far field by using an objective lens either in the transmission or collection mode (see Barer, Cosslett, eds 1990, Advances in Optical and Electron Microscopy. Academic; Betzig, 1992, Science 257: 189-95). The benefits of NSOM are its improved spatial resolution and the ability to correlate spectroscopic information with topographic data. The molecules of the array need to either have an inherent optically detectable characteristic such as fluorescence, or be labelled with an optically active dye or particle, such as a fluorescent dye. It has been proposed that resolution can be taken down to just a few nanometres by scanning apertureless microscopy (Scanning Interferometric Apertureless Microscopy: Optical Imaging at 10 Angstrom Resolution” F. Zenhausern, Y. Martin, H. K. Wickramasinghe, Science 269, p. 1083; T. J. Yang, G. A. Lessard, and S. R. Quake, “An Apertureless Near-Field Microscope for Fluorescence Imaging”, Applied Physics Letters 76: 378-380 (2000).

Alternatively excitation can be limited to the near field by a scanning probe or a narrow slit in near-field proximity to the sample. Acquisition may be in the far field (Tegenfeldt et al., 2001, Physical Review Letters 86: 1378-1381).

Far-Field Confocal Microscopy

In confocal microscopy, a laser beam is brought to its diffraction-limited focus inside a sample using an oil-immersion, high-numerical-aperture objective. The fluorescent signal emerging from a 50-100 μl region of the sample is measured by a photon counting system and displayed on a video system (for further background see Pawley J. B., ed 1995, Handbook of Biological Confocal Microscopy). Improvements to the photon-counting system have allowed single molecule fluorescence to be followed in real time (see Nie et al., 1994, Science 266: 1018-21). A further development of far-field confocal microscopy is confocal two-photon fluorescence microscopy, which can allow excitation at a single wavelength (see for example, Mertz et al., 1995, Opt. Lett. 20: 2532-34).

Wide-Field Epi-Illumination

The optical excitation system used in this method generally consists of a laser source, defocusing optics, a high performance dichroic beamsplitter, and an oil-immersion, low autofluorescence objective. Highly sensitive detection is achieved by this method using a cooled, back-thinned charge-coupled device (CCD) camera or an intensified CCD (ICCD). High-powered mercury lamps may also be used to provide more uniform illumination than is possible for existing laser sources. The use of epi-fluorescence to image single myosin molecules is described in Funatsu et al., 1995, Nature 374: 555-59.

Evanescent Wave Excitation

At the interface between a glass-liquid/air interface, the optical electromagnetic field decays exponentially into the liquid phase (or air). Molecules in a thin layer of about 300 nm immediately next to this interface can still be excited by the rapidly decaying optical field (known as an evanescent wave). A description of the use of evanescent wave excitation to image single molecules is provided in Hirschfeld, 1976, Appl. Opt. 15: 2965-66 and Dickson et al., 1996, Science 274: 966-69. The imaging setup for evanescent wave excitation typically includes a microscope configured such that total internal reflection occurs at the glass/sample interface (Axelrod D. Methods on Cell Biology 1989 30: 245-270). Alternatively a periodic optical microstructures or gratings can provide evanescent wave excitation at the optical near-field of the grating structures. This serves to increase signal around 100 fold (surface planar waveguides have been developed by Zeptosens, Switzerland; similar technology has been developed by Wolfgag Budach et al., Novartis Switzerland-poster at Cambridge Healthtech Institutes Fifth Annual meeting on “Advances in Assays, Molecular Labels, Signalling and Detection). Preferably an intensified CCD is used for detection.

Superresolution Far-Field Optical Methods

Superresolution far-field optical methods have been highlighted by Weiss, 2000 (PNAS 97:8747-8749). One new approach which merits mention is point-spread-function engineering by stimulated emission depletion (Klar et al 2000, PNAS 97: 8206-8210) which may improve far-field resolution by 10 fold. Distance measurement accuracy of better than 10 nm using far field microscopy, can be achieved by scanning a sample with nanometre size steps using a piezo-scanner (Lacoste et al PNAS 2000 97: 9461-9466). The resulting spots are localised accurately by fitting then to the known shape of the excitation point-spread function of the microscope. The laboratory of Enrico Gratton is developing similar measurement capabilities by circular scanning of the excitation beam. Shorter distances can typically be measured by molecular labelling strategies utlilising FRET [Ha et al Chem. Phys. 1999 247:107-118) or near field methods such as SPM. These distance measurement capabilities will be useful for the sequencing applications proposed in this invention.

Microarray Scanners

The burgeoning microarray field has introduced a plethora of different scanners based on many of the above described optical methods. These include scanners based on scanning confocal microscopy, TIRF and white light for illumination and Photomultiplier tubes, avalanche photodiodes and CCDs for detection. However, commercial array scanners in their standard form are not sensitive enough for SMD and the analysis software is inappropriate.

Studies have suggested that by varying the angle of incidence in TIRF microscopy it is possible to discriminate between fluorophores on a nanometric scale (Ajo-Franklin C M, Kam L, Boxer S G. PNAS 2001: 98 (24): 13643-8. This can lead to discrimination of closely spaced probes. A separate method for nanometric localization precision has been described by Thompson et al (Thompson R E, Larson D R, Webb W W. Biophys J. 82:2775-83).

Since the molecular arrays of the invention are spatially addressable, any immobilised molecule of interest/element of interest can be interrogated by moving the substrate comprising the array to the appropriate position (or moving the detection means). In this way as many or as few of the elements in the array can be read and the results processed. x-y stage translation mechanisms for moving the substrate to the correct position are available for use with microscope slide mounting systems (some have a resolution of 100 nm). Movement of the stage can be controlled automatically by computer if required. Ha et al (Appl.Phys. Lett. 70: 782-784 (1997)) have described a computer controlled optical system which automatically and rapidly locates and performs spectroscopic measurements on single molecules. A galvonometer mirror or a digital micromirror device (Texas Instruments, Houston) may be used to enable scanning of the image from a stationary light source. Signals can be processed from the CCD or other imaging device and stored digitally for subsequent data processing.

Multicolour Imaging

Signals of different wavelength can be obtained by multiple acquisitions or by simultaneous acquisition by splitting the signal, using RGB detectors or analysing the whole spectrum (Richard Levenson, Cambridge Healthtech Institutes Fifth Annual meeting on Advances in Assays, Molecular Labels, Signaling and Detection, May 17-18^{th} Washington D.C.). Several spectral lines can acquired by the use of a filter wheel or a monochromater. Electronic tunable filters such as acoustic-optic tunable filters or liquid crystal tunable filters can be used to obtain multispectral imaging (e.g. Oleg Hait, Sergey Smirnov and Chieu D. Tran, 2001, Analytical Chemistry 73: 732-739).An alternative method to obtain a spectrum is hyperspectral imaging (Schultz et al., 2001, Cytometry 43:239-247).

The Problem of Background Fluorescence

Microscopy and array scanning are not typically configured for single molecule detection. The fluorescence collection efficiency must be maximized and this can be achieved with high numerical aperture (NA) lenses and highly sensitive electro-optical detectors such as avalanche diodes that reach quantum yields of detection as high as 0.8 and CCDs that are intensified (e.g I-PentaMAX Gen III; Roper Scientific, Trenton, N.J. USA) or cooled (e.g. Model ST-71 (Santa Barbara Instruments Group, Calif., USA). However, the problem is not so much the detection of fluorescence from the desired single molecule (single fluorophores can emit ˜10⁸ photons/sec) but the rejection of background fluorescence. This can be done in part by only interrogating a minimal volume as done in confocal microscopy and TIRF. Traditional spectral filters can be applied to reduce the contribution from surrounding material (largely Rayleigh and Raman scattering of the excitation laser beam by the solvent and fluorescence from contaminants).

To reduce background fluorescence to levels which allow legitimate signal from single molecules to be detected a pulsed laser illumination source synchronized with a time gated low light level CCD can be used (Enderlein et al in: Microsystem technology: A powerful tool for biomolecular studies; Eds.: M. Köhler, T. Mejevaia, H. P. Saluz (Birkhäuser, Basel, 1999) 311-29)). This is based on the phenomena that after a sufficiently short pulse of laser excitation the decay of the analyte fluorescence is usually much longer (1-10 ns) than the decay of the light scattering (˜10² ps). Pulsing of a well chosen laser can reduce the background count rate so that individual photons from individual fluorophores can be detected. The laser power, beam size, and repetition rate, must be appropriately configured. A commercial array scanner and its software can be customized (Fairfield Enterprises, USA) so that robust single molecule sensitivity can be achieved.

In addition to these methods that combat fluorescence noise from within the sample volume, the instrument itself can contribute to background noise. Such tehrmoelectronic noise can be reduced for example by cooling of the detector. Coupling SPM measurements with optical measurements would be one way of correlating signals optically detected to the targeted structures rather than those due to other sources. Spatial or temporal correlation of signal from two probes targeting the same molecule suggests the desired rather than extraneous signal (e.g. Castro and Wiffiams Anal. Chem. 1997 69: 3915-3920).

Low fluorescence immersion oils should be used and substrates that are ultra-clean and of low intrinsic fluorescence. Glass slides/coverslips should be of high quality and well cleaned (e.g with detergents such as Alconex and Chromerge (VWR Scientific, USA) and high purity water)). Preferably fused quartz should be used which has a low intrinsic fluorescence. Single fluorophores can be distinguished from contaminating particules by several features: spectral dependence, concentration dependence, quantized emission and blinking. Particulate contaminants usually have broad spectrum fluorescence which is obtained in several filter sets whereas single fluorephores are only visible in specific filter sets.

The signal to noise ratio can also be improved by using labels with higher signal intensities such as fluospheres (Molecular Probes Inc.) or multilabelled dendrimers.

Label Free Detection.

A number of physical phenomena may be adapted for detection, that rely on the physical properties of the immobilised molecules alone or when complexed with captured targets or that modify the activity or properties of some other elements. Terahertz frequency is one way that the difference between double stranded and single stranded DNA could be detected Brucherseifer et al., 2000, Applied Physics Letters 77: 40494051. Interferometry, elliposometry, refraction would be other means. The modification of the signal from a light emitting diode integrated into the surface would be another means. The native electronic, optical (e.g. absorbance), optoelectronic and electrochemical properties would be other means. Various modes of the AFM could detect differences on the surface in a label free manner. The quartz crystal microbalance would be another means.

Coating DNA Between Electrodes with Metal and Measuring Conductivity

A method where the immobilised single molecule acts as a template for fabricating a nanowire where the single molecules are selectively coated with a material that facilitates detection. The coating is typically a conducting material which allows a circuit to form between only those electrodes which are occupied by the target molecule (by virtue of the target molecule binding to a the probes present on each of the electrodes). A potential difference is applied between electrodes in any two contiguous groups of electrodes and the electrodes on which probes interact with target are identified by virtue of the fact that a current flows between them. The conducting material can be from silver, gold, palladium and/or conjugated polymer. Where multiple single molecules span the electrodes then the haplotype frequency is given by the amount of current that flows between the electrodes. Protocols based on methods described in the following articles can be used: Richter J et al., 2001, Applied Physics Letters 78: 536-538; Braun E et al., 1998, Nature 391: 775; Quake S, and Scherer., 200, Science 290: 1536-1540.

C. Processing of Raw Data and Means for Error Limitation

Where Signals from Different Labels, such as Different Dyes, Overlap, the First Stage is Typically to Deconvolute the Signals if they Overlap.

Digital Analysis of Signals

Discrete groups of assay classification (e.g. nucleotide base calling) can be defined by various measures. A set of unique parameters are chosen to define each of several discrete groups. The result of interrogation of each individual molecule can be assigned to one of the discrete groups. One group can be assigned to represent signals that do not fall within known patterns. For example there may be groups for real base additions, a, c, g, and t in extension assays.

One of the prime reasons that single molecule resolution techniques are set apart from bulk methods is that they allow access to individual molecules. The most basic information that can be obtained is the frequency of occurrence of hits to a particular group. In bulk analysis the signal is represented in analogue by an (arbitrary) intensity value (from which a concentration may be inferred) and this indicates the result of the assay in terms of, say, a base call or it may indicate the level of a particular molecule in the sample, by virtue of its calibrated interaction profile. In contrast, the single molecule approach enables direct counting and classification of individual events.

Moreover, digital data processing facilitates error correction and temporal resolution of reactions at the array surface. Thus, time-resolved microscopy techniques may be used to differentiate between bona-fide reactions between probe and sample and “noise” due to aberrant interactions which take place over extended incubation times. The use of time-gated detection or time-correlated single-photon counting is particularly preferred in such an embodiment.

The invention accordingly provides a method for sorting signals obtained from single molecule analysis according to the confidence with which the signal may be treated. A high confidence in the signal will lead to the signal being added to a PASS group and counted; signals in which confidence is low are added to a FAIL group and discarded, or used in error assessment.

Table 1 illustrates the processing of signals for error analysis by example, for SNP typing by primer extension. The object of the process represented by the flowchart is to eliminate errors from the acquired image. The input for the process is one of the four colours (representing each of four differentially labelled ddNTPs) from the acquired image (after beam splitting). This process is performed on each of the four split signals.

Signals that satisfy a number of criteria are put into a PASS table. This PASS table is the basis for base calling after counting the number of signals for each colour.

The FAIL table is made so that information about error rate can be gathered. The five different types of errors can be collected into separate compartments in the FAIL table so that the occurrence of the different types of error can be recorded. This information may aid experimental methods to reduce error, for example it may reveal which is the most common type of error. Alternatively, the failed signals may be discarded.

The five criteria that are used to assess errors are:
1. If intensity is less than p where p=a minimum threshold intensity. This is high pass filter to eliminate low fluorescence intensity artefacts
2. If intensity is less than q, where q=a maximum intensity threshold. This is a lowpass filter to eliminate high fluorescence intensity artefacts.
3. If time is less than x where x=early time point. This is to eliminate signals due to self-prining which would be expected to occur early.

If time is greater than z, where z=late timepoint. This is to eliminate signals due to mis-priming of nucleotides which the enzyme would be expected to incorporate over an extended period. For example this may be due to priming by template on template which would be a two-step process, involving hybridisation of the first template to array and then hybridisation of the second template molecule to the forst template molecule.
4. Nearest neighbour pixels are compared to eliminate those in which signal is carried over multiple adjacent pixels which would be indicative of signals from, for example, non-specific adsorption of clumps or aggregates of ddNTPs.

The reaction is controlled by adjusting reaction components, for example salt concentration, ddNTP concentration, temperature or pH such that the incorporations occur within the time window analysed.

If a single dye molecule, which photobleaches after a defined time, is associated with each ddNTP, then an additional sub-process may be added which eliminates signals that occur in the same pixel over multiple time points.

If the array is composed of elements an additional process can be used to organise the data into groupings representing the array elements.

In the scheme described the system is configured such that a single pixel measures a single molecule event (statistically, in the large majority of cases). The system may be set up, for example, such that several pixels are configured to interrogate a single molecule.

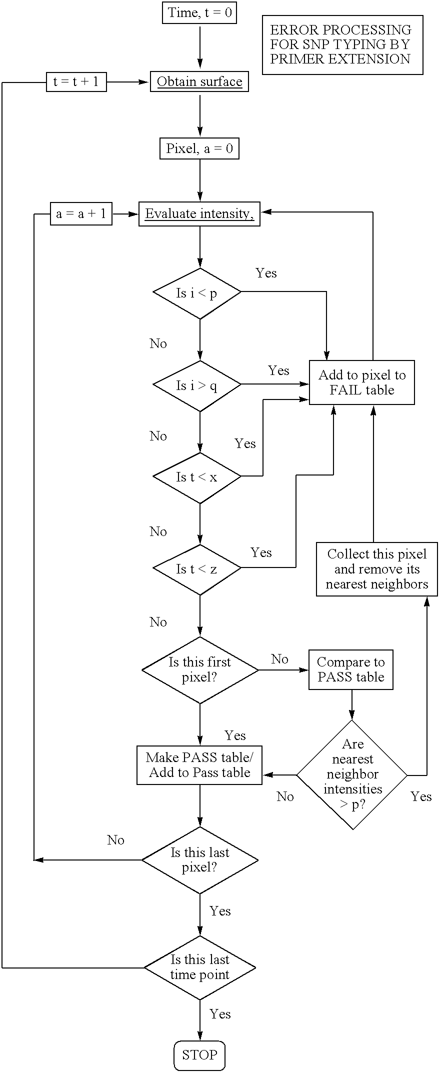

Thus, in a preferred embodiment, the invention relates to a method for typing single nucleotide polymorphisms (SNPs) and mutations in nucleic acids, comprising the steps of:
a) providing a repertoire of probes complementary to one or more nucleic acids present in a sample, which nucleic acids may possess one or more polymorphisms;
b) arraying said repertoire on to a solid surface such that each probe in the repertoire is resolvable individually,
c) exposing the sample to the repertoire and allowing nucleic acids present in the sample to hybridise to the probes at a desired stringency such that hybridised nucleic acid/probe pairs are detectable;
d) imaging the array in order to detect individual hybridised nucleic acid/probe pairs;
e) analysing the signal derived from step (d) and computing the confidence in each detection event to generate a PASS table of high-confidence results; and
f) displaying results from the PASS table to type polymorphisms present in the nucleic acid sample.

Preferably, the confidence in each detection event is computed in accordance with Table 1.

Advantageously, detection events are generated by labelling the sample nucleic acids and/or the probe molecules, and imaging said labels on the array using a suitable detector. Preferred labelling and detection techniques are described herein.

Methods for Reducing Errors

Single molecule analysis allows access to specific properties and characteristics of individual molecules and their interactions and reactions. Specific features of the behaviour of a particular molecular event on a single molecule may belie information about its origin. For enzymatic assays one example is that there may be a slower rate of mis-incorporations than correct incorporations. Another example is that there may be a different rate of incorporations for self-priming compared to priming in which the target forms the template. The rate characteristics of self-priming are likely to be faster than from priming of sample. This is because self-priming is a unimolecular reaction whereas priming of sample DNA is bimolecular. Therefore if time-resolved microscopy is performed, the time-dependence of priming can distinguish self-priming and mis-priming from correct sample priming. Alternatively, it might be expected that DNA priming form the perfectly matched sample has the capacity to incorporate a greater number of fluorescent dye NTPs in a multi-primer primer extension approach (Dubiley et al Nucleic Acids Research 1999 27: e19i-iv) than a mis-priming and a self-priming and so would give a higher signal level or molecular brightnesss. It may be difficult to differentiate between correct incorporation and mis-incorporation in the mini-sequencing (multi-base approach) because even though a wrong base may take longer to incorporate it may be associated with the primer for the same length of time as the correctly incorporated base. If the fluorescence intensity of a ddNTP in quenched to some degree when it is incorporated then the molecular brightness/fluorescence intensity may be a way of distinguishing between mis-incorporation which may take longer to become fixed than correct incorporation.

Different means for reduction of errors may be engineered into the system. For example, in genetic analysis, FRET probes can be integrated at the aflelic site. The conformation of a perfect match allows the fluorescent energy to be quenched whereas the conformation of a mismatch does not. The FRET probes may be placed on a spacer, which can be configured to accentuate the distances of FRET probes between matched and mismatched base pair sets.

Mismatch errors can be eliminated in some cases by degradation by enzymes such as RNAse1.

In addition to false positive errors discussed above, false negatives can be a major problem in hybridisation based assays. This is particularly the case when hybridisation is between a short probe and a long target, where the low stringency conditions required to form stable heteroduplex concomitantly promotes the formation of secondary structure in the target which masks binding sites. The effects of this problem may be reduced by fragmenting the target, incorporating analogue bases into target or probe, manipulating buffers etc. Enzymes can help reduce false negatives by trapping transient interactions and driving the hybridisation reaction forward (Southern, Mir and Shchepinov, 1999, Nature Genetics 21:s5-9). However, it is likely that false negatives will remain to some level. As previously mentioned, because large-scale SNP analysis without the need for PCR is enabled the fact that some SNPs do not yield data is not a major concern. For smaller scale studies, effective probes may need to be pre-selected.

In cases where the amount of sample material is low, special measures must be taken to prevent sample molecules from sticking to the walls of the reaction vessel and other vessels used for handling the material. These vessels can be silanised to reduce sticking of sample material and/or can be treated in advance with blocking material such as Denhardt's reagent or tRNA.

Alternative Methods for Detection and Decoding of Results

The molecules may be detected, as mentioned above, using a detectable label or otherwise, and correlating the position of the label on an array with information about the nature of the arrayed probe to which the label is bound. Further detection means may be envisaged, in which the label itself provides information about the probe which is bound without requiring positional information. For example, each probe sequence may be constructed to comprise unique fluorescent or other tags (or sets thereof), which are representative of the probe sequence. Such encoding could be done by stepwise co-synthesis of probe and tag by split and pool combinatorial chemistry. Ten steps generates every 10 mer encoded oligo (around 1 million sequences). 16 steps generates every 16 mer encoded oligos (around 4 billion sequences) which would be expected to occur only once in the genome. Fluorescent tags that are used for encoding could be of different colours or different fluorescent lifetimes. Moreover, unique tags may be attached to individual single molecule probes and used to isolate molecules on anti-tag arrays. The anti tag arrays may be spatially addressable or encoded.

D. Assay Techniques and Uses

A further aspect of the present invention relates to assay techniques based on single molecule detection. These assays may be conducted using molecular arrays produced by the methods of the invention or by any other suitable means.

The spatial addressable array is a way of capturing and organizing molecules. The molecules can then be assayed in a plethora of ways, including using any assay method which is suitable for single molecule detection, such as those described in WO0060114; U.S. Pat. No. 6,210,896; Watt Webb, Research Abstract: New Optical Methods for Sequencing Individual Molecules of DNA, DOE Human Genome Program Contractor-Grantee Workshop m, web page:www.oml.gov/hgmis/publicat/00santa/31.html on Feb. 5, 2001.

In general, the assay methods of the invention comprise contacting a molecular array with a sample and interrogating all or part of the array using the interrogation/detection methods described above. Alternatively, the molecular array is itself the sample and is subsequently interrogated with other molecules or probes using the interrogation/detection methods described above.

Many assay methods rely on detecting binding between immobilised molecules in the array and target molecules in the sample. However other interactions that may be identified include, for example, interactions that may be transient but which result in a modification to the properties of an immobilised molecule in the array, such as charge transfer.

Once the sample has been incubated with the array for the desired period, the array may simply be interrogated (following an optional wash step). However, in certain embodiments, notably nucleic acid-based assays, the captured target molecules may be further processed or incubated with other reactants. For example, in the case of antibody-antigen reactions, a secondary antibody which carries a label may be incubated with the array containing antigen-primary antibody complexes.

Target molecules of interest in samples applied to the arrays may include nucleic acids such as DNA and analogues and derivatives thereof, such as PNA. Nucleic acids may be obtained from any source, for example genomic DNA or cDNA or synthesised using known techniques such as step-wise synthesis. Nucleic acids may be single or double stranded. Other molecules include: compounds joined by amide linkages such as peptides, oligopeptides, polypeptides, proteins or complexes containing the same; defined chemical entities, such as organic molecules; combinatorial libraries; conjugated polymers, lipids and carbohydrates.

Due to the high sensitivity of the approach specific amplification steps can be eliminated if desired. Hence, in the case of analysis of SNPs, extracted genomic DNA can be presented directly to the array (a few rounds of whole genome amplification may be desirable for some applications). In the case of gene expression analysis normal cDNA synthesis methods can be employed but the amount of starting material can be low. Genomic DNA is typically fragmented prior to use in the methods of the present invention. For example, the genomic DNA may be fragmented such that substantially all of the DNA molecules are 1 Mb, 100 kb, 50 kb, 10 kb and/or 1 kb or less. Fragmentation may be achieved using standard techniques such as passing the DNA through a narrow guage syringe, sonication, alkali treatment, free radical treatment, enzymatic treatment (e.g. DNaseI), or combinations thereof.

Target molecules may be presented as populations of molecules. More than one population may be applied to the array at the same time. In this case, the different populations are preferred differentially labelled (e.g. cDNA populations labelled with Cy5 or Cy3). In other cases such as analysis of pooled DNA, each population may or may not be differently labelled.

A number of assay methods of the present invention are based on hybridisation of analyte to the single molecules of the array elements. The assay may stop at this point and the results of the hybridisation analysed.

However, the hybridisation events may also form the basis of further biochemical or chemical manipulations or hybridisation events to enable further probing or to enable detection (e.g. a sandwich assay). These further events include primer extension from the immobilised molecule/captured molecule complex; hybridisation of additional probes to the immobilised molecule/captured molecule complex and ligation of additional nucleic acid probes to the immobilised molecule/captured molecule complex.

For example, following specific capture (by hybridisation or hybridisation plus enzymatic or chemical attachment) of a single target strand by immobilised oligonucleotide, further analysis can be performed on the target molecule. This can be done on an end-immobilised target (or a copy thereof—see below) Alternatively, the immobilised oligonucleotide anchors the target strand which is then able to interact with a second (or higher number) of immobilised oligonucleotide(s), thereby causing the target strand to lay horizontally. Where the different immobilised oligonucleotide are different allelic probes for different loci, the target strand can be allelically defined at multiple loci.

The target strand can also be horizontalised and straightened, after being anchored by immobilised oligonucleotide by various physical methods known in the art.

In one embodiment, following hybridisation the array oligonucleotide can be used as a primer to produce a permanent copy of the bound target molecule which is covalently fixed in place and is addressable.

Single molecule counting of these assays will allow even a rare polymorphism/mutation in a homogeneous population to be detected.

Some specific assay configurations and uses are described below.

Nucleic Acid Arrays and Accessing Genetic Information

To interrogate sequence, in most cases the target must be in single stranded form. The exception would be cases such as triplex formation, binding of proteins to duplex DNA (Taylor J R, Fang, M M and S. Nie, 2000, Anal. Chem. 72:1979-1986), or sequence recognition facilitated by RecA (see Seong et al., 2000, Anal. Chem. 72: 1288-1293) or by the use of PNA probes (Bukanov et al, 1998, [PNAS] 95: 5516-5520; Cherny et al, 1998, Biophysical Journal 1015-1023). Also, the detection of mismatches in annealed duplexes by MutS protein has been demonstrated (Sun, HBS and H Yokoto, 2000, Anal. Chem 72: 3138-3141). Long RNAs (e.g. mRNA) can form R-loops inside linear ds DNA and this may be the basis for mapping of genes on arrayed genomic DNA. Where adouble stranded DNA target is arrayed, it may be necessary to provide suitable conditions to partially disrupt the native base-pairing in the duplex to enable hybridisation to probe to occur. This may be achieved by heating the surface/solution of the substrate, manipulating salt concentration/pH or applying an electric field to melt the duplex.

One preferred method for probing sequences is by probing double stranded DNA using strand invasion locked nucleic acid (LNA) or peptide nucleic acid (PNA) probes under conditions where transient breathing nodes in the duplex structure can arise, such as at 50-65° C. in 0-100 mM monovalent cation.

There are several methods that have been described to stretch out double stranded DNA so that it can be interrogated along its length. Methods include optical trapping, electrostatic trapping, Molecular Combing (Bensimon et al Science 1994 265: 20962098), forces within an evaporating droplet/film (Yokota et al Anal. Biochem 1998 264:158-164; Jing et al PNAS 1998 95: 8046-8051), centrifugal force and moving the air-water interface by a jet of air (Li et al Nucleic Acid Research 1998 6: 4785-4786).

Molecular Combing which involves surface tension created by a moving air-water interface/mensicus and a modification to the basic technique has been used to stretch out several hundred haploid genomes on a glass surface (Michalet et al Science. 1997 277: 1518-1523).

Relatively fewer methods have been described for single-stranded DNA Woolley and Kelly (Nanoletters 2001 1: 345-348) achieve elongation of ssDNA by translating a droplet of DNA solution linearly across a mica surface coated with positve charge. The forces exerted on ssDNA are thought to be from a combination of fluid flow and surface tension at the travelling air-water interface. The forces within fluid flow can be sufficient to stretch out a single strand in a channel. Capillary forces can be used to move solutions within channels.

DNA can be combed onto a surface by one of the standard methods (eg as described by Bensimon et al., above) and this is followed by processes to acquire genetic or sequence information form the single molecules.

In a preferred embodiment, however, the combing on the surface is performed as follows:

Oligonucleotide probes are spread and fixed randomly on the surface of the substrate. The DNA to be combed is then captured from solution, by hybridisation to these oligonucleotides. Following capture the DNA is combed onto the surface. In a preffered embodiment the DNA is combed by flow of fluids over the surface. The combed DNA can optionally be dehydrated and fixed on the surface. This is shown schematically in FIG. 8.

Methods described herein for hybridisation to single or double stranded DNA can be used. For “capture combing” of Lambda DNA, oligonucleotides complementary to either of the “sticky” ends of Lambda phage DNA can be used. Similarly, genomic DNA can be captured by digesting it with specific restriction enzymes and to use oligonucleotide capture probes complementary to the overhangs which are generated. The advantage of this capture combing for randomly immobilsing nucleic acids on a surface is that it enables a very homogeneous spread of the DNA on substantially all of the surface. This is in contrast to the patchy coverage typically achieved by standard combing methods.

Moreover, specific probes can be used which allow combing of only specific desired types of nucleic acids from a complex mixture of nucleic acids. For example if the capture probes is oligo d(T) it specifically capture s polyadenylated mRNA

These methods, in addition to stretching out DNA, overcome intermolecular secondary structures which are prevalent in ssDNA under conditions required for hybridisation.

An alternative way of overcoming secondary structure formation of nucleic acids on a surface would be by heating the surface of the substrate or applying an electric field to the surface.

The the majority of the assays described below do not require the molecules to be linearised, as positional information along the molecules length is not required. In the cases where positional information is required, DNA needs to be linearised/horizontalised. The attachment to more than one surface immobilised probe will facilitate the process. Double stranded targets can be immobilised to probes having sticky ends such as those created by restriction digestion.

In one embodiment, following capture by an immobilised oligonucleotide, a target strand is straightened. This can be done on a flat surface by molecular combing. In one embodiment the probes could be placed on a narrow line on the left most side of an array element and then the captured molecules would be stretched out in rows form left side to the right side by moving an air-water interface from left to right.

Alternatively the captured target can be stretched out in a channel or capillary where the capture probes are attached to (one or more) walls of the vessel and the physical forces within the fluid cause the captured target to stretch out. The target molecules could be stretched out thus by methods that do not rely on probe capture, instead an oligo that is 5′ phosphorylated can be made to attach to appropriately derivatised surfaces under acidic pH conditions. These conditions may be created with fluid flow within a channel/capillary to immobilise and stretch out a target strand. Fluid flow may facilitate mixing and this would make hybridisation and other processes more efficient. Reactants could be recirculated within the channels during the reactions.

Although in a number of embodiments described below, interrogation of multiple sites of a target nucleic acid is achieved by separate binding of multiple copies of that target nucleic acid to various elements in the array, in one embodiment, a single nucleic acid molecule may be simultaneously interrogated at multiple loci by binding to multiple elements suitably spatially placed (the construction of arrays with a suitable layout is described in section A). This type of detection may conveniently be applied to SNP detection, haplotyping and sequence determination. Various aspects are discussed below under individual headings but are typically broadly applicable to any detection technique where simultaneous interrogation of a single molecule at multiple sites is desired.

1. Resequencing and/or Typing of Single-Nucleotide Polymorphisms (SNPs) and Mutations

a. Hybridisation

The organisation of the array would typically follow the known art as taught by Affymetrix e.g. Lipshutz et al., Nature Genetics 1999 21: s20-24; Hacia et al., Nature Genetics 21: s42-47) ) for SNP resequencing or typing. In short, an SNP may be analysed with a block of array elements containing defined probes, in the simplest form, with probes to each known or possible allele. This may include substitutions and simple deletions or insertions. However, whereas the Affymetrix techniques require complex tiling paths to resolve errors, advanced versions of the single molecule approach may suffice with simpler arrays, as other means for distinguishing errors may be used. Transient interactions can also be recorded.

Typically the oligonucleotides will be between about 17 and 25 nucleotides in length although longer or shorter probes may be used in some instances.

In a different implementation, a mix of probes complementary to all alleles is placed within a single array element. Each probe comprising a different allele is distinguishable from the other probes, e.g. each single molecule of a particular allele will have a specific dye associated with it. A single molecule assay system of the invention allows this space saving operation and would be simple to do when pre-synthesised oligos are spotted on the array.

The probe can be appended with a sequences that would promote its formation into a secondary structure that would facilitate the discrimination of mismatch (e.g. a stem loop structure where the probe sequence is in the loop).

The following are typical reaction conditions that can be used: 1M NaCl or 3-4.4 M TMACl in Tris Buffer, target sample, 4 to 37° C. in a humid chamber for 30 mins to overnight.

It is recognised that hybridisation of rare species is discriminated against under conventional reaction conditions, whilst species that are rich in A-T base pairs are not able to hybridise as effectively as G-T rich sequences. Certain buffers are capable of equalising hybridisation of rare and A-T rich molecules, to achieve more representative outcomes in hybridisation reactions. The following components may be included in hybridisation buffers to improve hybridisation with positive effects on specificity and/or reduce the effects of base composition and/or reduce secondary structure and/or reduce non-specific interactions and/or facilitate enzyme reactions:

1M Tripropylamine acetate

N, N-dimethylheptylamine

1-Methyl piperdine

LiTCA

DTB

C-TAB

Betaine

Guanidinium isothyacyanate

Formamide

Tetramethy ammonium chloride (TMACl)

Tetra ethyl Ammonium Chloride (TEACl)

Sarkosyl

SDS (Sodium dodecyl sulphate)

Dendhardt's reagent

Poly ethyene Glycol

Urea

Trehalose

Cot DNA

tRNA

N-N-dimethylisopropylamine acetate.

Buffers containing N-N-dimethylisopropylamine acetate are very good for specificity and base composition. Related compounds with similar structure and arrangement of charge and/ or hydrophobic groups may also be used.

Probes are chosen, where possible, to have minimal potential for secondary structure and cross hybridisation with non-targeted sequences.

Where the target molecules are genomic DNA and specific PCRs are not used to enrich the SNP regions of choice, measures need to be taken to reduce complexity. The complexity is reduced by fragmenting the target and pre-hybridising it to C₀t=1 DNA. Other methods are described by Cantor and Smith (Genomics, The Science and Technology Behind the Human Genome Project 1999; John Wiley and Sons]. It may also be useful to perform whole genome amplification prior to analysis.

The probes would preferentially be morpholino, locked nucleic acids (LNA) or peptide nucleic acids (PNA).

Molecules and their products may be immobilized and manipulated on a charged surface such as an electrode. Applying an appropriate bias to the electrode may speed up hybridization and aid in overcoming secondary structure when the bulk solution is at high stringency. Switching polarity would aid in preferentially eliminating mismatches.

b. Stacking Hybridisation

Adding either sequence specific probes or a complete set of probes in solution that will coaxially stack onto the immobilised probe, templated by the target, can increase the stability and specificity of the hybridisation. There is a stability factor associated with stacking and this is abrogated if there is a mismatch present between the immobilised probe and the solution probe. Therefore mismatch events can be distinguished by use of appropriate temperatures and sequence.

The probe can be appended with sequences that configure it to form a secondary structure such that it provides a coaxial stacking interface onto which the end of a target is juxtaposed. This may be a favourable approach when the target is fragmented.

It may be advantageous to use LNA probes as these may provide better stacking features due to their pre-configured “locked” structure.

The following are typical reaction conditions that can be used: 1M NaCl in Tris Buffer; 1 to 10 nM (or higher concentration) stacking oligonucleotide; target sample; 4-37° C. 30min to overnight

c. Primer Extension

This is a means for improving specificity at the free end of the immobilised probe and for trapping transient interactions. There are two ways that this can be applied. The first is the multiprimer approach, where as described for hybridisation arrays, there are separate array elements containing single molecules for each allele.

The second is the multi-base approach in which a single array contains a single species of primer whose last base is upstream of the polymorphic site. The different alleles are distinguished by incorporation of different bases each of which is differentially labelled. This approach is also known as mini-sequencing.

The following reaction mix and conditions can be used: 5× polymerase buffer, 200 mM Tris-HCL pH 7.5, 100 mM MgCl₂, 250 mM NaCl 2.5 mM DTT; ddNTPs or dNTPs (multibase); dNTPs (multiprimer), Sequenase V.2 (0.5 μ/μl) in polymerase dilution buffer, target sample, 37° C. degrees 1 hr.

d. Ligation Assay

Ligation, (chemical or enzymatic) is another means for improving specificity and for trapping transient interactions. Here the target strand is captured by the immobilised oligonucleotide and then a second oligonucleotide is ligated to the first, in a target dependent manner. There are two ways that this can be applied. In the first type of assay, the “second” oligonucleotides that are provided in solution are complementary in the region of the known polymorphisms under investigation. One oligo of either the array oligos or the “second” solution oligonucleotide will overlap the SNP site and the other will end one base upstream of it.

In the second type of assay, the second oligonucleotides in solution comprise the complete set, every oligonucleotide sequence of a given length. This would allow analysis of every position in the target. It may be preferable to use all sequences of a given length where one or more nucleotides are LNA.

A typical ligation reaction is as follows: 5× ligation buffer, 100 mM Tris-HCL pH 8.3, 0.5% Triton X-100, 50 mM MgCl, 250 mM KCl, 5 mM NAD+, 50 mM DTT, 5 mM EDTA, solution oligonucleotide 5-10 pmol. Thermus thermophilus DNA ligase (Tth DNA ligase) 1 U/ul, target sample, between 37° C. and 65° C. 1 hr.

Altematively, stacking hybridisation can be performed first in high salt: 1M NaCl, 3-4.4M TMACl, 5-10 pmol solution oligonucleotide, target sample.

After washing of excess reagents from the array under conditions that would retain the solution oligonucleotide, the above reaction mix minus solution oligonucleotide and target sample is added to the reaction mix.

Combining the Power of Different Assay Methods

The power of primer extension and ligation can be combined in technique called gap ligation (the processivity and discriminatory power of two enzymes combined). Here a first and a second oligonucleotide are designed that hybridise in close proximity on the target but with a gap of preferably a single base. The last base of one of the oligonucleotides ends one base upstream or downstream of the polymorphic site. In cases where it ends downstream, the first level of discrimination is through hybridisation. Another level of discrimination occurs through primer extension which extends the first oligonucleoitde by one base. The extended first oligonucleotide now abuts the second oligonucleotide. The final level of discrimination occurs where the extended first oligonucleotide is ligated to the second oligonucleotide.

Alternatively the ligation and primer extension reactions described in c. and d. above can be performed simultaneously, with some molecules of the array giving results due to ligation and others giving results due to primer extension, within the same array element. This would be a way to increase confidence in the base call, being made independently by two assay/enzyme systems. The products of ligation may be differetly labelled than the products of primer extension.

The primer or ligation oligonucleotides may be designed on purpose to have mismatch base at a site other than the base that serves to interrogate the polymorphic site. This would serve to reduce error as duplex with two mismatch bases is considerably less stable than a duplex with only one mismatch.

It may be desirable to use probes that are fully or partially composed of LNA (which have improved binding characteristics and are compatible with enzymes) in the above described enzymatic assays.

The invention provides a method for SNP typing which enables the potential of genomic SNP analysis to be realised in an acceptable time-frame and at affordable cost. The ability to type SNPs through single-molecule recognition intrinsically reduces errors due to inaccuracy and PCR-induced bias which are inherent in mass-analysis techniques. Moreover, if errors occur which left a percentage of SNPs untyped, assuming errors are random with regard to position of SNP in the genome, the fact that the remaining SNPs are typed without the need to perform individual (or multiplexed) PCR still confers an advantage. It allows large-scale association studies to be performed in a time- and cost-effective way. Thus, all available SNPs may be tested in parallel and data from those in which there is confidence selected for further analysis.

There is a concern that duplicated regions of the genome may lead to errors, where the results of an assay may be biased by DNA from a duplicated region. The direct assay of the genome by single molecule detection is no more susceptible to this problem than assays utilising PCR since in most instances the way PCR is commonly designed, a small segment surrounding the SNP site is amplified (this is necessary to achieve multiplex PCR). However, with the availability of the genome sequence, this would be less of a problem as in some cases it may be possible to select non-duplcated regions of the genome for analysis. In other cases, the sources of bias would be known and so could be accounted for.

If signal is obtained from probes or labels representing only one allele then the sample is likely to be homozygous. If it is from both, in substantially a 1:1 ratio then the sample is likely to be heterozygous. As the assays are based on single molecule counting, highly accurate allele frequencies can be determined when DNA pooling strategies are used. In these case the ratio of molecules might be 1:100. Similarly, a rare mutant allele in a background of the wild-type allele might be found to have ratio of molecules as 1:1000.

Tagging Mismatches

As an alternative means for selecting SNPs or mutation, is to detect the sites of mismatches when a heterozygous sample DNA (one or both of which contain 2′-amine subsitiuted nucleotides) is denatured and re-annealed to give heteroduplexes can be tagged 2′ amine acylation (or more preferably an unknown sample DNA can be hybridised to modified tester DNAs of known sequence. This is made possible by the fact that acylation occurs preferably at flexible positons in DNA and less preferably in double stranded constrained regions (John D and K Weeks, Chem. Biol. 2000, 7: 405410). This method could be used to place bulky tags onto sites of mismatch on DNA that has been horizontalised. Detection of these sites may then be detected by for example AFM. When this is applied genome-wide the genome would be sorted by array probes or the identity of fragments obtained by use of encoded probes.

Homogeneous Assays

Low background fluorescence and the elimination of the need for post-assay processing to remove unreacted fluorescent labels can be achieved by two approaches. The first is the use of Molecular Beacons (Tyagi et al Nat. Biotechnol. 1998, 16:49-53) and other molecular structures comprising Dye-Dye interactions in which fluorescence is only emitted in the target bound state and is quenched when the structure is unbound by the target. In practice a fraction of the molecular beacons will fluoresce and so an image may need to be taken before adding targets to the array to make a record of false positives.

The second is the analysis of fluorescence polarization of a dye labelled molecule (Chen et al Genome Res. 1998, 9: 492-98). For example, in a mini-sequencing assay, free and incorporated dye labels exhibit different rotary behaviour. When the dye is linked to a small molecule such as a ddNTP, it is able to rotate rapidly, but when the dye is linked to a larger molecule, as it would be if added to the primer by incorporation of the ddNIP, rotation is constrained. A stationary molecule transmits back into a fixed plane, but rotation depolarises the emitted light to various degrees. An optimal set of four dye terminators are available where different emissions can be discriminated. These approaches can be configured within single molecule detection regimes. Other homogeneous assays are described by Mir and Southern (Ann Rev. Genomics and Human Genetics 2000, 1: 329-60). The principles inherent in pyrosequencing (Ronaghi M et al Science, 1998, 363-365) may also be applicable to single molecule assays.

2. Haplotyping

Two or more polymorphic sites on the same DNA strand can be analysed. This may involve hybridisation of oligos to the different sites but each labelled with different fluorophores. As described, the enzymatic approaches could equally be applied to these additional sites on the captured single molecule.

In one embodiment each probe in a biallelic probe set may be differentially labelled and these labels are distinct from the labels associated with probes for the second site. The assay readout may be by simultaneous readout by splitting of the emission by wavelength obtained from the same foci or from a focal region defined by the 2-D radius of projection of a a DNA target molecule immobilised at one end. This radius is defined by the distance between the site of immobilized probe and the second probe. If the probes from the first biallelic set are removed or their fluors photobleached then a second acquisition can be made with the second biallelic set which in this case do not need labels that are distinct from labels for the first biallelic setin another embodiment haplotyping can be performed on single molecules captured on allele-specific microarays. Haplotype information can be obtained for nearest neighbour SNPs by for example, determining the first SNP by spatially addressabe allele specific probes (see FIG. 7a). The labelling is due to the allelic probes (which are provided in solution) for the second SNP. Depending on which foci colour is detected within a SNP 1 allele specific spot determines the allele for the second SNP. So spatial position of microarray spot determines the allele for the first SNP and then colour of foci within the microarray spot determines the allele for the second SNP. If the captured molecule is long enough and the array probes are far enough apart then further SNP allele specific probe, each labelled with a different colour can be resolved by co-localization of signal to the same foci.

More extensive haplotypes, for three or more SNPs can be reconstructing from analysis of overlapping nearest neighbour SNP haplotyes (see FIG. 7b) or by further probing with differently labeled probes on the same molecule.

Samples molecules may be pre-processed to bring distal sites into closer vicinity. For example this can be done by appropriate modular design of PCR or ligation probes. For example, the modular ligation probe would have a 5′ sequence that would ligate to one site and the 3′ portion would have a sequence that would ligate at a distal site on the target. Use of such modular probes would juxtopose two distal elements of interest and cut out the intervening region that is not of interest.

In the case where the target has been horizontalised, the labels associated with the first locus need not be distinct from labels associated with subsequent loci; the position specifies the identity.

In another embodiment of the invention, single nucleic acid molecules may be simultaneously multiply probed by suitable spatial placement of probes at distinct locations. For example, four SNPs could be interrogated, each 10 kb apart along a 40 kb DNA molecule. The ˜3 micron spacing of these SNPs could be replicated in the spacing of patches of probes on the surface that would interact with the SNPs. If all SNPs, which would occur every ˜1000 bp then the spacing of SNPs and probes on the surface is 300 nm. Moreover, each allele of the SNP would be represented in cells, one above the other and the series of probes against consecutive SNPs on the taget molecule would run sequentially from left to right (or right to left) on the surface. Here the alleles (hence haplotype) present on a single molecule may be revealed by looking at the target strand on the surface. For example it may be complementary to probes on the bottom for the first two SNPs but complementary to the top positions at the third SNP and fourth SNP as shown in FIG. 1A (see FIG. 1B for an alternative path). By tracing the path taken by the strand, which is guided by hybridisation to perfect complement on the surface(see FIG. 1C) the haplotype can be obtained. The target DNA strand could be directly visualised by AFM or may be labelled with a fluorescent dye e.g YO-YO or TO-TO dyes Molecular Probes Inc. and analysed by optical microscopy. An alternative, and one which would be conducive to an integrated device would be to place each probe on a nanoelectrode, use redox mediators in solution and then measure the change in cyclic voltametry or other electronic meaure to indicate hybridisation. The target strand would trace a path along upper or lower electrodes depending on which allele is present on the strand. Hybridisation with a single probe molecule on the electrode would be detected through charge transfer to the nanoelectrode for example. The footprint due to the path of the DNA strand would be revealed by the spatial location of the electrodes that give signal. Alternatively, and as described above, the DNA molecules can serve as template for deposition of conducting materials and subsequent determination of through which electrode-probe pairs current can flow due to a circuit being made.

Where the probes are labelled with a detectable label, such as a fluorophore, the haplotype is given by the spatial coordinates of the fluorescent footprint. The patch of probes may be of high density but only the single immobilised molecule that interacts with the single target molecule would be a finctionally active molecule of the array. It would be possible to obtain haplotype frequencies by this method in two ways. Firstly haplotype frequencies of nearest neighbour SNPs could be obtained where multiple single molecules occupy the patch sets. A haplotype of greater than two nearest neighbours would be difficult to obtain as there may be crossover of molecules. The second way of obtaining haplotype frequencies would be to have multiple copies of the patch sets on the surface which each interrogate a single molecule only.

A limitation of DNA pooling methods for genotyping is that because individual genotypes are not analysed, the estimation of haplotypes is complicated. However, in the methods described in the present invention, DNA pooling strategies could be used to obtain Haplotype frequencies.

3. Fingerprinting

A captured target strand can be further characterised and uniquely identified by further probing by hybridisation or other means. The particular oligonucleotides that associate with the target strand provide information about the sequence of the target. This can be done by multiple acquisitions with similarly labelled probes (e.g. after photobleaching or removal of the first set) or simultaneously with differentially labelled probes. A set of oligonucleotides, which are differentially labelled could be specifically used for simultaneous fingerprinting.

Again, individual molecules may be simulataneously multiply probed as described for haplotyping.

4. Nucleic Acid Sequencing

Capture of DNA molecules would be the basis for complete or partial sequence determination of the target by various means. The captured DNA can be sequenced by determining interactions by Watson-Crick base pairing, serially to a complete set of sequences, e.g. every 6-mer.

For example, a mixture of two or more probes could be placed within the array element. The plating density would be such that individual probe molecules would be sufficiently spaced to capture a single molecule at defined points. Alternatively, two or more probes could be placed at defined positions within an array element, as a means to stretch out target DNA by hybridisation to these probes. The horizontal molecule could then be characterised by, for example, using fluorescent probes or tagged probes (as described below). Each array element would address an individual fragment from the genome. This could form the basis of resequencing the genome using SPM or a high resolution optical method. If the array has one million sites, then it will typically be necessary to fragment human genomic DNA into 3000 bp lengths to cover the entire genome. For 50,000 element array 60 kb fragments would cover the entire genome. The method for sequencing and sequence reconstruction is given section below.

The target DNA may be substantially a double stranded molecule and probing may be by strand invasion with PNA or LNA. Hybridisation at around 50° C. would be sufficient to create single stranded nodes within the duplex which would seed strand invasion. A salt concentration between 0 and 1 M Na would typically be appropriate for PNA. A salt concentration between 50 mM and 1 M Na would typically be appropriate for LNA.

The target may be substantially single stranded but would be made accessible to hybridisation by stretching out on a surface. This may be achieved by passing the molecules through a channel that makes a seal with the substrate and passing a solution of the molecules through by capillary action.

Other methods of obtaining sequence information described herein would be applicable to sequence analysis on probe-captured DNA.

Sequence information could be obtained by probing along a single molecule using blocks of probe arrays in a similar manner to that described above for haplotyping. Multiple copies of each sequence would typically be required and probes would typically need to be laid out in optimal spatial locations to obtain sequence information. The position of individual molecules over the array containing known sequences would need to be determined.

The present invention also relates to methods of arraying pluralities of nucleic acid molecules at low density where, although the identity of the nucleic acids may be unknown prior to immobilisation, the array is subsequently characterised by the use of encoded probes, such as tagged probes. Or by successive serial hybridization/melting of each probe from a complete repertoire e.g around a thousand cycles with 5 mers and then reconstructing the sequence from information about the probes that hybridise to each immobilized nucleic acid. In addition to obtaining sequence from a sample nucleic acid this could also be a way of randomly arraying probes eg 25 mers and then making the sequences spatially addressable by decoding their sequence by hybridisaiton with shorter probes.

5. STR Analysis

The array oligonucleotide could probe the sequence flanking a repetitive element. This captures a sequence containing a repetitive element. It is then used to seed ligation of probes complementary to the repetitive sequence, along the target strand or to act as primer to polymerise a complementary strand to the repetitive elements. Then the number of repeat units are determined by quantitating the level of signal from fluorescently labelled oligonucleotides or fluorescent nucleotides. Only completely extended oligos which incorporate an oligo (preferably by stacking hybridisation or ligation) complementary to the other flanking sequence labelled with a different fluorophore, are typically counted. It may be helpful to obtain ratios between fluorescence intensity from the extended region and the labelled flanking sequence Ligation conditions described above (see lc) can be used; a reaction temperature of 46-65° C. with a thermostable ligase is preferable. Polymerisation conditions described above can be employed.

A method to determine repeat lengths based on providing probes complementary in length to the different target repeat lengths as described (Case Green et al, p61-67 DNA Microarrays A Practical Approach Ed: M. Schena1999 Oxford University Press) can also be implemented at the singe molecule level.

6. Expression Analysis

Conventional microarray expression analysis is performed using either synthetic oligonucleotide probes (e.g 40-75 nt) or longer cDNA or PCR product probes (typically 0.6 kb or more) immobilised to a solid substrate. These types of arrays can be made according to present invention at low surface coverage (as described in section A). After hybridisation, the level of gene expression can be determined by single molecule counting using the methods of the invention. This will give increased sensitivity and will allow events due to noise to be distinguished from real events. Also, as the basic unit of counting is the single molecule, even a rare transcript can be detected. One implementation of expression analysis involves comparison of two mRNA populations by simultaneous analysis on the same chip by two-colour labelling. This can also be done at the single molecule level by counting each colour separately by for example beam splitting. Capture of a target cDNA or mRNA can allow further analysis by oligonucleotide probing. For example this could be used to distinguish alternatively spliced transcripts.

Microarray Theory Suggests that Accurate Gene Expression Ratios at Equilibrium can be Obtained when the Sample Material is Low.

A permanently addressable copy of an mRNA population can be made by primer extension of molecules separated on single molecule arrays. Primers could be designed based on the available genome sequence or gene fragment sequences. Alternatively, unknown sequences could be sampled using a binary probe comprising a fixed element that would anchor all mRNA and a variable element that would address/sort the repertoire of mRNA species in a population. The fixed element may be complementary to sequence motifs that are common to all mRNA such as the Poly A sequence or the Polyadenylation signal AAUAAA or preferably to a common clamp sequence that is ligated to all mRNA or cDNA at 5′ or 3′ ends. The copy could be used as the basis for further analysis such as sequencing.

7. Comparative Genomic Hybridisation (CGH).

Gridded genomic DNA or genomic DNA immobilized by spatially addressable capture probes (or complementary copies) is probed by genomic DNA from a different source to detect regions of differential deletions and amplifications between the two samples. The immobilized sample containing multiple copies of each species may be a reference set and genomic DNA from two different sources may be differentially labeled and compared by hybridization to the reference.

8. Detection of Target Binding to a Repertoire of Oligonucleotides

A target can be hybridised to a repertoire of ligands. Single molecule analysis would be advantageous for example it would reveal binding characteristics of conformational isomers and overcome the steric hindrance associated with binding of targets to arrays in which molecules are tightly packed. Hybridisation would be conducted under conditions close to those that would occur in the intended use of any selected ligand.

For antisense oligonucleotide binding to RNA, hybridisation would occur at 0.05 to 1 M NaCl or KCl with MgCl₂ concentrations between 0 and 10 mM in for example Tris Buffer. One picomole or less of target will be sufficient. (Refer to EP-A-742837: Methods for discovering ligands).

The method also provides a method for randomly arraying a combinatorial repertoire. Such a repertoire could allow billions of molecules to be analysed in an assay that would be designed to detect a signal from each molecule by single molecule detection techniques. The encoding would identify the molecule. The combinatorial repertoire, whether it is encoded or not could be made much more simply than conventional libraries e.g by adding a mixture of all four bases at every step of synthesis in DNA synthesis as is done when generating repertoires for systematic evolution of ligands by exponential enrichment (SELEX)(Tuerk C and Gold L., Science 1990 249: 5050-510) However because analysis is at the single molecule level, enrichment by PCR is mot needed for detection. In application to nucleic acid structures or aptamers, after a functional assay such as a binding assay, the immobilized target molecule could be probed by short oligonucleotides to determine it's sequence by sequence by hybridsatin methods. Molecular combing would facilitate this.

9. Protein-Nucleic Acid Interactions

Interactions between biological molecules, such as proteins, and nucleic acids can be analysed in a number of ways. Double stranded DNA polynucleotides (by foldback of designed sequences) can be immobilised to a surface in which individual molecules are resolvable to form a molecular array. Immobilised DNA is then contacted with candidate proteins/polypeptides and any binding determined by the methods described above. Alternatively RNA or duplex DNA can be horizontalised and optionally straightened by any of the methods refered to herein. The sites of protein binding may then be identified within a particular RNA or DNA using the methods described herein. Candidate biological molecules typically include transcription factors, regulatory proteins and other molecules or atoms such as calcium or iron. When binding to RNA is analysed meaningful secondary structure is typically retained.

The binding of labeled transcription factors or other regulatory proteins to genomic DNA immobilized and linearised by the methods referred to herein may be used to identify active coding regions or the sites of genes in the genome. This would be an experimental alternative to the bioinformatic approaches that are typically used to find coding regions in the genome. Similarly, methylated regions of the genome could be denoted by using antibodies specific for 5-methylcytosine. Differential methylation may be an important means for epigenetic control of the genome, the study of which is becoming increasingly important. Information from tag probes would preferably be combined with information about methylated regions and coding regions.

Below is out of place in protein-nucleic acid interactions as it is talking about interactions of AFM tip with methylated DNA, is it still OK in this section?:

An alternative means for determining the methylation status of DNA would be by force or chemical force analysis using AFM. For example a silicon nitride AFM tip would interact differently with methyl cytosine in DNA, which is more hydrophobic than non-methylated DNA.

Other applications include RNA structure analysis and hybridisation of tags to anti-tag arrays.

Other Types of Assays

The present invention is not limited to methods of analysing nucleic acids and interactions between nucleic acids. For example, in one aspect of the invention, the molecules are proteins. Capture probe may be used to bind protein. Other probes can further interrogate protein. For example, further epitopes may be accessed by antibodies or an active site by a small molecule drug.

Low density molecular arrays may also be used in methods of high-throughput screening for compounds that interact with a given molecule of interest. In this case, the plurality of molecules represent candidate compounds (of known identity). The molecule of interest is contacted with the. array and the array interrogated to determine where the molecule binds. Since the array is spatially addressable, the identity of each immobilised molecule identified as binding the molecule of interest can be readily determined. The molecule of interest may, for example, be a polypeptide and the plurality of immobilised molecules may be a combinatorial library of small molecule organic compounds.

Many of the above assays involve detecting interactions between molecules in the array and target molecules in samples applied to the array. However, other assays include determining the properties/characteristics of the arrayed plurality of molecules (even though their identity is already known), for example determining the laser induced fluorescence characteristics of individual molecules. An advantage over bulk analysis would be that transient processes and functional isomers would be detected.

Thus in summary, the assays of the invention and the low density molecular arrays of the invention may be used in a variety of applications including genetic analysis, such as SNP detection, haplotyping, STR analysis, sequencing and gene expression studies; identifying compounds/sequences present in a sample (including environmental sampling, pathogen detection, genetically modified foodstuffs and toxicology); and high through screening for compounds with properties of interest. High throughput genetic analysis will be useful in medical diagnosis as well as for research purposes.

Advantages of the single molecule array approach can be summarised as follows:
1. Can resolve complex samples.
2. Can separate correct signals from erroneous signals.
3. Sensitivity of detection down to a single molecule in the analyte.
4. Sensitivity of detection of a single variant molecule within a pool of common (e.g. wild-type) molecules.
5. Eliminates need for sample amplification.
6. Allows individual molecules in target sample to be sorted to discrete array elements and to ask specific questions of said target molecules e.g. analyse multiple polymorphic sites (i.e. haplotyping).
7. Can perform time-resolved microscopy of single molecular events within array elements and hence detect transient interactions or temporal characteristics of single molecule processes.
8. Due to single molecule counting can get very precise measurements of particular events e.g. Allele frequencies or mRNA concentration ratios.
   E. Alternative Assay Methods

A further aspect of the invention relates to the production of arrays comprising randomly immobilised molecules from a sample of interest. These arrays are then interrogated to obtain information about the immobilised molecules in the array. This approach is typically applied to pluralities of polypeptide or nucleic acids obtained from, for example cells, in genomics or proteomics approaches. Not only will characterisation of the arrays provide useful genomic and proteomic information about the sample which has been arrayed, but characterised arrays may then be used in many of the methods described above.

One method for obtaining a signature identity for each molecule within a randomly immobilised array is surface enhanced raman spectroscopy (SERS). A single molecule can be attached to a colloidal gold or silver bead, and the beads spread on a surface. This enables the raman signal due to the single molecule to be enhanced sufficiently for it to be detected. Raman spectroscopy is advantageously carried out within a scanning probe microscopy configuration. This kind of Raman spectroscopy may further provide some structural information about the molecules under investigation

Moreover, Raman spectroscopic fingerprints can be used for encoding labels for probes as required for certain aspects of the present invention (see Cao, Y C, Rongchao, J and C A Mirkin, Science 297:1536-1540 2002).

Where immobilization is totally random, there will be a poisson distribution of molecules on the surface, spaced apart at a variety of distances. Some molecules will be too close to resolve by optical microscopy. It can thus be advantageous to make the distribution of a non-spatially addressable random array ordered such that each molecule occupies a fixed distance from any other. For example, each individual molecule can be positioned at a pre-defined position by using for instance scanning probe microscopy. Another method involves attaching the molecules to binding sites created in two or three dimenensional lattices of the type described by Winfree et al. (Nature 1998 394:53944.)

Proteomics—Immobilisation of Target Molecules and Interrogation of Physicochemical Properties

In an alternative method for characterization of the protein content of a cell or tissue, the sample molecules are not captured by array molecules. Instead the sample is applied to a solid phase (lacking, not comprising, an immobilised array of molecules), with each individual molecule settling randomly on the surface. For example, protein molecules can be adsorbed onto a variety of surfaces, with some proteins better adapted for one surface than another. The surface could be differentially patterned with different surface coatings e.g. hydrophilic or hydrophobic. Then individual protein molecules are differentiated by their size, shape, mass or any physicochemical property, preferably by scanning probe microscopy. They may also be differentiated due to the region of surface attachment on an array of different surface chemistries.

Proteins may also be recombinant. There are currently efforts underway to make a catalogue of clones so that any protein can be expressed off the shelf. Hence each protein (and any variant) can be expressed individually, placed on the surface and its characteristics determined or “learnt” by the method, preferably based on SPM. To analyse the >40,000 protein molecules refers to the minimum which is due to the number of genes; must be a higher number of proteins due to alternative splicing and post-translational modifications] in a high throughput manner to learn their characteristics, an array format may be useful, and it is likely that arrays of increasing numbers of different proteins will become increasingly available . A method for producing an in vitro array of proteins by in cell free synthesis from PCR products has been reported (M He and M Taussig Nucelic Acid Research 2001 29: e73

Then for a complete description of the protein content of a cell, questions such as what proteins (and variants) are present, how many individual molecules of each are present, and which proteins are interacting with which other proteins can be asked. If learning has only been performed on a subset then it is only the members of the subset that can be identified. Any proteins that do not fit the description of a “learnt” protein can be stored in computer memory along with its determined characteristics for future identification. Such unknown proteins may become implicated with particular functions due to being correlated with specific expression or prevalence patterns in different biological or pathological situations.

This approach could be extended to look at other components of cells or tissues such as lipids, polysaccharides or the metabolome.

Genomics—Immobilisation of Target Molecules and Interrogation with Tagged Probes.

In an alternative means for haplotyping and for sequencing, the sample nucleic acids are not captured by array probes. Instead the sample (e.g. fragmented genomic DNA) is applied to a solid phase (lacking not comprising an immobilised array of molecules), with each individual molecule settling randomly on the surface and becoming horizontalised. For example DNA molecules can be adsorbed to mica surfaces in the presence of certain divalent cations, e.g. nickel or cobalt or magnesium or onto polylysine coated surfaces. The use of low pH promotes attachment of molecules only by one end. The molecules would then preferably be straightened by methods known in the art and as discussed above. The method of application of the nucleic acids may also lead to straightened molecules. The targets may be in double or single stranded form as discussed above.

The identities of individual molecules can be determined by probes of known sequence. Sixteen nucleotides of sequence information are typically required to identify uniquely a DNA fragment in the genome. It would be expected that this length of sequence information would allow the fragment to be mapped to the genome. Only 7 to 9 nucleotides may be sufficient to uniquely tag mRNA. Preferably, the identity of each molecule is encoded prior to arraying (by pre-hybridisation of the sample DNA with the repertoire of tags).

Obtaining 16 nucleotides of sequence information from one or more proximal points allows each molecule to be identified. For example, four 4 mers would give the requisite information and would require only 256 different tags. Six 3 mers would give 18 nt of sequence information and this would require only 64 tags, although it would be difficult to obtain stable hybrids with such short length. These oligonucleotide sizes could be incorporated into methods described herein for synthesizing complementary strands by ligation. Or alternatively the short oligonucleotides could be analogues that bind with greater strength such as PNA, LNA and Morpholino oligos.

Zhong et al, 2001 (PNAS 98: 3940-3945) and Woolley et al., 2000 (Nature Biotechnology 18: 760-763) have demonstrated analysis of haplotypes on single molecules. The methods of the present invention would similarly obtain haplotypes in molecules that are not captured by array probes. However, the methods disclosed herein differ from Zhong et al, 2001 and Woolley et al., 2000 in that the molecules are probed with two distal tagged probes to uniquely identify a strand and SNPs analysed in between these two tagged probes by using dual labelled biallelic probes.

The entire sequence of individual molecules can be determined by probing with a complete set of oligonucleotides. This can be done sequentially with each individual oligonucleotide of the set. Or it can be done simultaneously where each probe sequence of the set is encoded. It is advantageous to discriminate mismatches of one or two bases (this can be done by controlling hybridisation and wash stringencies, use of enzymes, chemical cleavage of mismatches etc). However, highly exacting discrimination of mismatches is not essential and may be tolerated depending on the approach used for sequence reconstruction. In a population of molecules there would be many copies of each sequence present. Each position may be probed by multiple sequences and even mismatches, which usually behave predictably can be informative. If the oligonucleotides are short (e.g. 6 mers) then their complementary sequences are likely to occur at multiple positions along the length of a single molecule. However, the positional information and/or information of order of probes along molecule, that can be obtained by analysing horizontalised single molecules would be highly useful in re-assembling the sequence. The software/algorithms used for sequence reconstruction may be similar to those developed for Sequencing by Hybridisation (SbH) by for example Pavel Pevzner of the University of Califronia.

The process typically involves: addition of an oligonucleotide, recording if it interacts with the target molecule and determining its location relative to the ends of the target molecule and relative to positions occupied by other probes; denaturation of oligonucleotide from the target molecule (e.g. by heating, manipulating salt concentration or pH or by applying an appropriately biased electric field); and probing with the next oligonucleotide. There may be many copies of each target molecule (unless the sample material is unamplified genomic DNA from a single cell) and so results from each copy would add to the confidence in the reconstruction.

Application of positive charge or electric field to the chip surface would be expected to facilitate horizontalisation. This could be done by adding positive charges by chemical treatment of the surface or by applying an positive bias. Hybridisation would also be facilitated by electric fields, when much of the hybridisation volume distal to the surface is kept at high stringency to eliminate secondary structure in the target, but the polarity of the electric field applied in the vicinity of the probe would serve to attract DNA and to screen the negative backbones of the DNA target and probe to enable hybridisation to occur. Flipping to opposite polarity would serve to remove mismatches preferentially.

Double stranded DNA could be analysed using strand invasion by PNA or LNA probes as described above.

Complementary Strand Synthesis by Ligation

The target may be probed (tagged) and made double stranded prior to immobilisation. A complementary strand is usually synthesised by DNA polymerase or reverse transcriptase. If each base that is incorporated could be identified then the sequence could be obtained. However current techniques are not sensitive enough to identify individual bases. In the method of the invention a complementary strand to a target single strand is synthesised by concatenation and ligation of oligonucleotides along the DNA (see FIG. 3). The reason for doing this is to incorporate. into the DNA chain, oligonucleotides which can be individually detected and uniquely identified as will be discussed below.

Typically around 250 nts can be efficiently synthesised by ligating 9 mers. However, further optimisation would be expected to increase the length that is possible to synthesize by ligation.

Gap Fill Ligation

Long stretches of DNA may be better catered for by using “gap fill ligation”. Here, rather than ligation of adjacent/contiguous oligonucleotides, oligonucleotides are placed apart and the gap between then is filled by template directed polymerisation of a complementary strand, primed from the 3′ side of each oligonucleotide, and abutting and terminating at the next oligonuceleotide. The polymerised strand is then ligated to the 5′ abutting end of the next oligonucleotide. This abutting oligonucleotide will itself have primed polymerisation toward the next olignucleotide along the chain and so on (see FIG. 4).

Such an approach would be useful for haplotyping. It will also be possible to use this approach for sequencing, where as described previously a complete library of oligonucleotides are used, each oligonucleotide being used in a concentration that allows attachment of oligonucleotides at roughly the desired distances form each other on the DNA chain, and each target DNA sequence being present in many copies. Sequence will be reconstructed from obtaining tag and positional information concerning the different sub-set of probes from the complete set that associate with each of the copies. The reaction is optimised so that enough overlapping sequence information is achieved by probing each of the copies to reconstruct the sequence.

Alternatives to Immobilisation to a Flat Surface

In addition to immobilisation of the target single DNA molecule on a flat surface, the DNA could be wrapped around a bead or particle or a nanorod or nanobar or it could be freely floating in solution. The tags that are associated with the beads would then be read by a sensitive flow cytometry system. Furthermore the strands could be flowing in channels or capillaries. Readout would typically be by far-field optical acquisition. Excitation could be through a near-field slit as described by Tegenfeldt et al., 2001, Physical Review Letters 86: 1378-1381

Tagging Schemes for Single Molecule Analysis

Analysis can be performed with one or a few different dyes (or other tags) if each oligonucleotide is applied sequentially. However, to maximise throughput in the system it would be desirable to probe with many or all oligonucleotides simultaneously. Hence there is a need to have a repertoire of tags corresponding to the repertoire of oligonucleotide sequences. A range of dye molecules are currently in use and these can be easily detected and can be differentiated. However, clearly there are not enough spectrally resolvable single dye molecules available to encode an entire repertoire of oligonucleotide sequences, even for say every 4 mer which numbers 256. One means to increase the available repertoire is by measuring fluorescent lifetimes as well as wavelength. For example Lanthanide containing fluorophores have around 5-50K fold longer lifetimes than ordinary fluors. But this will still not give enough unique tags to code for a useful number of oligonucleotides. Another means is measurement of molecular brightness which varies with number of dye molecules or particles of a particular wavelength. One solution is to use combinations of tags (dye molecules) to encode each oligonucleotide in the repertoire. Image acquisition may be by a system of the type described by Harold Garner and colleagues Schultz et al., 2001, Cytometry 43:239-247where a complete emission spectrum is obtained from each molecule, hence eliminating the complication of using filters to acquire multiple fluorescent wavelengths. Alternatives to dyes, especially for higher resolution analysis are labels that can be detected by STM or various forms of electron microscopy. These may be conducting materials or molecules. Also there would be a wide variety of tags that could be used if analysis was by AFM. Tags bearing any physicochemical property that is detectable by AFM could be used. For example, Ishino et al., (Jpn. J. Appl. Phys. 1995 33: 4718-4722) have demonstrated the discrimination of a series of charged functional groups by AFM. For encoding, these functional groups would need to be tethered to the oligonucleotide probes but spatially far apart from each other so that they can be individually detected by AFM; it would be desirable to use sharpened AFM probes or thin-walled carbon-nanotube probes. Alternatively and more preferably, each functional group from the palette is used to derivatise nanobeads. The oligonucleotides of the repertoire would be encoded by combinations of the derivatised nanobeads. AFM can be used to produce force curves which display the force-distance relationship of the AFM tip and substrate. Different physico-chemical features contribute to different shapes of the force curves. A single functional group could derivatise nanobeads at different surface coverages, for example 5% covered to 100% covered, and this density of coverage would be reflected in the force curves that are obtained. Different force-distance relationships would be found if the chemical nature of either the tip or the sample were changed. Chemical force microscopy involves derivatising the tip with known chemistries, including specific oligonucleotide sequences. An array of force curves can be obtained by Force Mapping (Heinz W F and Hoh J H, 1999, Biophysical Journal 76: 528-538).

The repertoire of tags could be created by using mass encoding. Each tag would have a different mass. This may be detected by scanning probe mass spectrometry as proposed by, for example, workers at Lancaster and Loughborough Universities (Pollock HM And Somg M, UK SPM meeting 2001, University of Leeds 10^{th} and 11^{th} April, Poster). A range of mass tags and various means for their construction have been reported by Shchepinov et al (e.g Tetrahedron 56: 2713 (2000)). Compounds of different masses can be directly coupled to the oligonucleotide by an asymmetric synthon. To generate large repertoire mass tags can be combined on beads or on the arms of dendrimers.

Dendrimers are useful structures for encoding, able to form a repertoire of nanostructures that can be discriminated by SPM for example or serve as arms or receptacles for holding other encoding entities.

Electrophore tags have also been described by Aclara Inc.

Luminex corp load polystyrene beads with different ratios of 20 or more dyes for encoding. Currently around 1000 different combinations can be produced but they aim to produce one million in the future. However, the size of beads they use for this are too large for single molecule sequencing as described above although they may be useful for determining haplotypes as SNP occur roughly every 1000/1200 bases (around 300-400 nm). However, if the appropriate dye dynamic range could be obtained from beads with a few nanometre dimensions, then these would be useful for encoding oligonucleotides for single molecule analysis. Fluorescently labelled latex nanoparticles are available from Sigma. Polystyrene beads loaded with dyes are available from molecular probes and these are available in sizes as small as 20 and 40 nm. Different surface chemistries are available for linking oligonucleotides including, carboxyl groups and streptavidin. Features of good beads would be non-leakiness of the dyes. Semiconductor nanocrystals or quantum dots (QDs) whose spectral emmision is narrow can be found from around 1.5 nm upwards. However the current sets of QDs that have been used rely on size of particle to control the emission wavelength (this is a quantum effect). This suggests that to obtain a repertoire, some QDs may be larger than the ˜3 nm size which will best fit with say 9 mer oligonucleotides. This would be satisfactory for haplotype analysis but not for sequencing. However, QDs of the same size but made of different semiconductor materials would be expected to emit at distinct wavelengths, so a series of sizes from 1.5 to 3 nm and a series of semiconductor materials can be used as the palette from which to build encodings. The QDS can be linked along a linear chain and spatial origin of signal from each could be determined or the spectral characteristics of the combination could be deconvoluted. Diversity could be increased by covering the QDs or dye loaded nanoparticles with a different characteristic that can be measured e.g. different types of functional groups or different densities of functional groups as described. Detection in this case would be by dual SNOM and AFM, various configurations of which have been described. AFM can also be combined with Far-field optical methods (e.g. Kolodny et al., 2001, Anal Chem. 73: 1959-1966). Similarly PRPs or SERS nanoparticles can be employed in many of these ways. Recently quantum nanobars have been reported. These have useful spectral signatures and also would be good substrates for linking further encoding schemes. The nanobars can be made of composite materials and so have different spectral characteristics in different regions. SurroMed Inc suggest that as many as 24,421,875 different Nanobarcodes could be created by nanobars composed of 11 stripes of 5 different metals (Remy Cromer, at Cambridge Healthtech Institutes Fifth Annual meeting on Advances in Assays, Molecular Labels, Signaling and Detection, May 17-18^{th} Washington D.C.)

Alternative methods would be required to resolve spectral lines if they a numbered around a million different lines. Interferometrc techniques may be capable of resolving this high number of spectral lines.

Another means for encoding is to use different lengths of an aliphatic chain. These must be perpendicular to the DNA chain. For example the DNA chain could be in a channel as has been described, the tag-chains could bear paramagnetic particles that are attracted to magnets that are placed parallel to the DNA, causing the chains to align perpendicular to the DNA. Or they could be aligned by an electric field. AFM can distinguish lengths very accurately. The tag chain could be electronic/metallic wires which are interrogated with STM or they could be coated with material that is easy to detect by AFM, e,g, by lateral force measurement. Polymerases can incorporate bases with for example biotin attached so the chains could be attached at appropriate positions on oligonucleotides or even on individual nucleotides. The chain could be very narrow and in this case if linked to individual bases they could allow base by base sequencing. This kind of tagging of individual nucleotides would allow linear reading of sequence without the need for sequence reconstruction. The tagging chain could in some instances be DNA, preferably restricted in alphabet (Mir K U. A Restricted Genetic Alphabet for DNA Computing In DNA Based Computers II, Publisher: American Mathematical Society (1998 ), which could be covered specifically with cytochrome and/or metal as done in electron microscopy.

Steric interference from tags could be reduced by using an adaptor encoding DNA sequence. For example, an oligonculeotide probe could be attached to a “surrogate” sequence tag which would bind to a specific anti-tag on the encoded bead.

Building up the Repertoire of Tagged Oligonucleotides

Oligonucleotides can be linked to their tags in two ways. Firstly, oligonucleotides and tags can be prepared separately and then manually linked together (not combinatorially). Secondly they can be joined by combinatorial chemistry by various means. Split and mix synthesis would be particularly appropriate. But rather than perform this on beads which is the usual way, this should be done directly by using an asymmetric synthon that can initiate both the oligonucleotide and the tag synthesis by stepwise solid-phase synthesis, each of olignucleotides and the tags having different protection groups (special protected sythons may need to be produced for the tags). Alternatively, say for the differential loading of dye or functional groups, the nature and position of each base would need to be encoded, Each base addition would correspond to a different level of loading of an entity of a particular type. For example, the identity of the base added could be encoded in the density of surface coverage of the bead by a particular functional group.

A=0 surface coverage

C=33% surface coverage

G=66% surface coverage

T=100% surface coverage

For the second and subsequent base additions a separate functional group, distinct from previous functional group(s) would be added, the identity of the functional group would indicate its location along the chain (is it first or seventh base for example). The density of surface coverage would indicate which base is added. The same logic can be followed for optical encoding, mass encoding etc.

Where the encoding is inherent in beads (or chains of beads), the beads could serve as templates for synthesis whilst orthogonally being labelled with appropriate encoding at each step. This would result with an encoded bead with many copies of the probe sequence on its surface. For single molecule analysis only one molecule would be required to interact with the target. The remaining molecules could be left redundant or inactivated. For example 99% of the molecules on the surface could be cleaved off and discarded. Alternatively, bead derivation chemistry would be done in away such that the stoichiometry of bead versus functional group (for initiating oligonucleotide synthesis) would be such that statistically only one or very few functional groups would associate with one bead.

Analysing Chromosomes/Fibre Fish

The tagging schemes described above could be used to haplotype or sequence directly on metaphase chromosomes by derivatives of FISH (Fluorescent in situ hybridisation). Here the genome would come in a pre-fractionated state, partitioned in the 46 chromosomes of a diploid cell. Landmarks that are visible by staining, further aid the positioning analysis. Proximal probes that cannot be resolved by location can be resolved by the encoding. The tags are typically separated from the probes by long linkers. Because of the condensed state of the DNA it is difficult to get access to the DNA. DNA in interphase nuclei is more accessible. The most accessible systems are DNA fibers (Fiber FISH) and nuclear (Weigant) halo preparations where the DNA is in a more extended form (Zhong et al., 2001, PNAS 98: 3940-3945). In Weigant halo preparations nuclei are prepared and treated so that DNA is de-proteinized and exploded from the nucleus. In these interphase DNA or naked DNA preparations, chromosomal information is lost.

Access with PNA, LNA, DNG (Linkletter et al., 2001, Nucleic Acids Research 29: 2370-2376) or Morpholino derivatives are expected to be better than DNA.

In general there is less restriction on the size of beads/tags for haplotyping (hundreds of nm to a few microns) but beads/tags must be of a few nm dimensions for sequencing applications. Similarly, the resolution of optical methods for reading fluorescent tags need not be high for haplotyping but must be high for sequencing. When the association of adjacent fluorescent tags to the DNA can be temporally resolved, then resolution can be improved by deconvolution algorithms.

A number of new tagging schemes are proposed that would be particularly useful for single molecule analysis. Such tagging schemes can be applied to other embodiments described in this invention and to other applications not related to single molecule analysis.

The immobilisation/tagging/encoding procedures described above may be used to generate randomly immobilised arrays of nucleic acid molecules, whose identity need not be known prior to immobilisation. The molecules are encoded such that they can be uniquely identified by hybridising a plurality of tagged probes as described above. Hybridisation of tagged probes may be conducted before or after immobilisation. Typically, the nucleic acid molecules are fragmented genomic DNAs or cDNAs.

Experimental Procedures

Primary Arrays

Primary arrays are those that carry the molecular species that are directly involved in molecular technique of the invention.

Preparations for Arraying

Arrays may be made by spotting one or more probes or sample molecules onto specific locations on a surface or by spreading probes or sample molecules onto a surface.

Cleaning Substrates

The following procedures are preferably performed in a clean room. The surface of a glass slide (e.g Knittel Glazer, Germany) or spectrosil slides is thoroughly cleaned. For example, Sonicate in a surfactant solution (2% Micro-90) for 25 minutes, wash in deionised water, rinse thoroughly with milliQ water, immerse in 6:4:1 milliQ H₂00:30%NH₄OH:30% H₂O₂ or in a H2SO4/CrO₃ cleaning solution for 1.5 hr. Rinse and store in dust free environment e.g under milliQ water. The top layer of Mica substrates are cleaved by covering with scotch tape and rapid pulling off of layer.

Slides

It was found that slides from several manufacturers were compatible with single molecule detection. It was found that slides from different suppliers varied in the quality of evanescent field that can be formed. We found that slides from Asper Biotech (Tartu, Estonia) produced a good evanescent field.

Slide Surface Chemistry

Three different slide chemistries ,Epoxysilane, Aminosilane and enhanced aminosilane (3-Aminopropyltrimethoxysilane+1, 4-Phenylenediisothiocyanate) have been tested. Single molecule arrays can be obtained with all three chemistries. Aminosilane surface coating can be used both for experiments which look at molecules as point sources of fluorescence as well as experiments which look at linearised DNA polymers therefore has been a preferred substrate. Enhanced aminosilane slides or polyelectrolyte coated slides are preferable when enzymatic reactions are performed

Derivatization of Glass with Polyethylenimine (PEI)

A glass slide is washed with 0.1 N acetic acid, then rinsed with water until the water rinsed from the slide has a pH equal to the pH of the water being used to rinse the slide. The slide is then allowed to dry. To a 95:5 ethanol:water solution is added a sufficient quantity of a 50% w/w solution of trimethoxysilylpropyl-polyethylenimine (600 MW) in 2- to achieve a 2% w/w final concentration. After stirring this 2% solution for five minutes, the glass slide is dipped into the solution, gently agitated for 2 minutes, and then removed. The glass slide is dipped into ethanol in order wash away excess silylating agent. The glass slide is then air dried. Aminated oligonucleotides are spotted in a 1 M sodium borate pH 8.3 based buffer or 50% DMSO.

Similar Polyelectrolyte coated slides may be purchased from VBC-Genomics (Austria).

Printing

Each sequence or molecular identity is placed at a specific spatial location on a surface so that a specific known molecular identity can be found by going to a particlar location on the surface and conversely by determining the coordinates of a location it is possible to determine the identity of molecules present therein.

Spotting Pins

Capillary pins from Amersham Biotech optimized for Sodium Thiocyanate buffer or pins optimized for DMSO buffer were used in different spotting runs. Both type of pins enabled single molecule arrays to be constructed. Other preferred spotting methods are the Affymetrix ring and pin system and ink jet printing. Spotting Pins have also been used (Kaken, Japan)

Determining Optimal Spotting Concentration for Making Spatially Addressable Single Molecule Arrays.

The first step in the procedure for making a single molecule microarrays is to do a dilution series of fluorescent oligonucleotidenculeotides. This has been done with 13 mers and 25 mers but any appropriate length of oligonucleotide can be chosen. These oligonucleotides may be aminated and preferably Cy3 labelled at the 5′ end.

10 uM solution of the oligonucleotide (this procedure is also appropriate to proteins and chemical spotting) is placed in a first well of the microtitre plate. For a 10 fold dilution, 1 ul is transferred into the next well of the microtitre plate and so on over several orders of magnitude (twelve orders of magnitude were tested. A 1:1 volume of 2× spotting buffer that is being tested is added to each well. This gives 5 uM concentration in the first well, 500 nM in the second well and so on. The array is then spotted using a microarrayer (Amersahm Generation III). The Dilution series is then analysed by TIRF microscpy or AFM or other relevant microscopy system, The morphology of spot is analysed and the distribution of molecules within the spot determined. The spot range with the desired number of resolvable single molecules is chosen. Optionally a further more focused dilution series is created around the dilution of interest. For example two 50% dilutions in the range 500 nM to 50 nM can be done.

In a first experiment a dilution series over 12 orders of magnitude was spotted with 4 buffers to establish the range of dilutions necessary. Subsequently more focused dilutions series are used. It was found that between 250 nM to 67.5 nM gave resolvable single molecules within an identifiable spot (if there are too few molecules then it is difficult to know exactly where the spot is. This will not be a problem when spot position and morphology is know to be regular and movement of translation stage or CCD is automated and is not manual). Some spots give a faint ring around the perimeter, this can help identify spots

To achieve single molecule array a dilution series of modified and unmodified oligonucleotides in several different spotting buffers on three different slide chemistries, on slides from several different manufacturers, two different humidities and using several different post-spotting protocols were tested. Due to the effects of photobleaching, the amount of pre-exposure to light will also influence the number of single-dye labeled single molecules that can be counted.

On enhanced aminosilane slides, QMT buffer 1, 1.5 M Betaine 3×SSC gave the best results. A faint ring was seen around the spots in 1.5 M Betaine 3×SSC. Concentrations between 250 nM and 67.5 nM were appropriate for single molecule counting on relatively fresh slides. These slides should be stored at −70. At room temperature the ability to retain probe after spotting wanes badly over a 2 month period.

Preparing Single Molecule Oligonucleotide Arrays

Oligonucleotide Chemistry

Unmodified DNA olignucleotides and oligonucleotidenucletides that were aminated at the 5′ or 3′ end were tested. There appears to be no significant difference in morphology or attachment whether the oligonucleotides are terminally modified or not. Several different sequences, of varying lengths that probe TNF alpha promoter have been tested. Thiol terminated nucleic acids can be spotted onto gold surfaces or mercaptosilane coated surfaces.

Spotting, from microtitre plates to slide, normal terminally aminated phospodiester oligonucleotides(Eurogentec, Belgium) are used.

Make arrays as above but employ oligonucleotides in which one more base is an LNA base (Proligo). 0.2 uM scale synthesis is sufficient to print thousands of arrays, alternatively for a large number of elements the arrays are more economic to make by combinatorial synthesis). Arrays can also be made by spotting PNA oligonucleotides (Oswel, UK or Boston Probes, USA).

Arraying Buffers

In total 11 different buffers have been tested. From the study it has emerged that the best general buffer on the APTES slides supplied by Asper Biotech is 50% DMSO and 50% Water. This buffer gives far superior spot morphology than any other buffer that was tested. Spotting humidity affects the morphology. Spotting was tested at 43%/42% and 53-55% humidity with both conditions giving useable arrays. However, there is a slight dougaut effect at 43% humidity compared to the almost perfect homogeneity at 55% humidity. QMT2 (Quantifoil, Jena Germany) buffer also give reasonable spots on Asper's Epoxysilane slides.

After spotting the epoxysilane slide is placed 15 minutes at 97 degrees C (this step may be omitted) and RT storage for 12 hours to 24 hours. This is followed by storage at 4 degrees C. overnight or preferably longer). The slides are washed before use. Two methods of washing work well. The first is washing 3× in miliQ water at room temperature. The second is washing on the Amersham Slide Processor (ASP). The following wash protocol was used.

Asp Wash Protocol

HEAT To 25 degrees

MIX Wash 1, (1×SSC/0.2%SDS) 5 or 10 minutes

PRIME Prime with wash 2(0.1×SSC/0.2%SDS)

FLUSH Wash 2

MIX Wash 2 30 seconds or 1 minute

FLUSH Wash 3 (Wash (0.1×SSC)

MIX Wash 3 30 seconds or 1 minute

PRIME Prime with was 4 (0.1×SSC)

FLUSH Wash 4 (0.1×SSC)

Prime Prime with Isopropanol

Flush Flush with Isopropanol

Flush Flush with air

Airpump Dry Slide

Heat Turn off Heat

The best buffers on the more expensive enhanced aminosilane (3-Aminopropyltrimethoxysilane+1, 4-Phenylenediisothiocyanate) slides from Asper Biotech are 50%1.5 M Betaine 50% 3×SSC and 10%QMT1 spotting buffer(Quantifoil, Jena). In addition some of the other buffers from Quantifoil (Jena, Germany) performed reasonably well; with testing of different concentrations of these buffers better morphology might be achievable. Detailed internal morphology seen with epi was not good. DMSO buffer (Amersham) gave intense “sunspots”, ie a dot of intense fluorescence, within the spots; it is conceivable that single molecules can be counted in the rest of the spot, ignoring the sunspot. Spotting was tested at 43% and 55% humidity with both conditions giving useable arrays.

For the enhanced aminosilane slides post-processing involves optional 2 hours at 37 degrees in humid chamber (more molecule stick but sometimes the spots can come out of line or merge and so this step is preferably avoided or the spots are arryed far enough apart to prevent merger). This is followed by overnight (or longer) at 4 degrees C. The slides are then dipped in 1% Ammonia solution for 2-3 minutes. The slides are then washed 3× in milliQ water and then put at 4 degrees C. overnight. There is some degree of bleeding of dye from the spots after hybridization. This may be addressed by more stringent or longer washing.

If the buffers in the microtire wells dry out, they can be resuspended again in water. The betaine buffer did not perform well when this was done.

50% DMSO is the best buffer for aminoslinae slides. After spotting these slides are immediately crosslinked with 300 mJoules on a Stratagene Crosslinker. The arrays are washed in hot water with shaking twice for two minutes and are then dunked five times in 95% ethanol and immediately dried with forced air. Substantialy more aminated oligonucleotides stick to the surface with this slide chemistry than other slide chemistries. Therfore less oligonucleotide needs to be spotted to get a particular surface density.

The spotting buffers produce significant autofluorescence in the green range which must be removed for accurate single molecule counting. This can be substantially removed by washing, especially with buffers containing detergents such as SDS and Sarkosyl. Alternatively, the green range of the spectrum is avoided, opting for probes which fluoresce in the red range, for example.

Spreading

Arrays can be made in which the location of the molecule does not specify the identity of the molecule until the molecules are sequenced or an encoding is decoded.

This type of array is also characterised by the fact that single molecules of the same identity are not necessarily found in the same region but are arranged randomly i.e. Sequence A may be adjacent to Sequence B and a second occurance of Sequnece A may be at a distal location from the first occurance. This random arrangement of the molecular species is due to the method used for making the array. Although having the molecules in such a arandom location does not confer any advantages, the fabrication of this type of array is far simpler than the fabrication of an array where many molecules of the same species are found in the same region on the surface as is the case for DNA colonies/Polonies or DNA microarrays.

This random aspect is a feature of many types of surface immobilised arrays. For example, Dynamic Molecular Combing (Michalet et al) produces random arrays, in vitro cloning (Chetverin et al) produces random arrays and so on.

We describe particular ways of making random arrays which are particularly suitable for single molecule applications described in this document. Firstly it is very simple to make a random array of any molecule of interest simply by spreading it out on a surface to which it interacts/binds or to which it adsorbs. For example, proteins can adsorb onto various type of surfaces. DNA can electrostatically bind to surfaces bearing positive charges etc, hence genomic DNA can be extracted and binds to aminosilane coated surface (see figure). Furthermore, It is almost as simple to make a pool of oligonucleotide in which the sequence at one or more positions is randomised. This can be done by providing mixes of the nucleotides during synthesis. Such a pool can be easily spread out on a surface to provide an array in which molecules of each species are distributed at random locations. Spreads may be of molecules which are viewed as a single point source of fluorescence.

Alternatively the molecules may be horizontalised and may be visualized as polymers. A procedure for horizonalising and substantially straightening molecules is as flollows: between 10 and 100 ul of sample (e.g Lambda DNA at a concentration of 500 ng/ml is placed between two microscope coverslips (24×60 mm, Matsunami Japan) in either TE Buffer pH 8 or HEPES/EDTA buffer pH 8. One surface is removed from the other by a lateral motion, optionally excess material is removed from the surfaces. Random arrays of straightened polymer are now created on both of the two flat surfaces. This method produces very good distributions of molecules as compared to many other combing methods where typically it is difficult to produce homogeneous molecular combing. The molecules of a secondary array (see below) can also be straightened/linearised in this way.

The following is another procedure for horizontalising straigtening DNA. Add a 30 ul drop at one end of the slide at the center. Use a forced air canister (Air Duster, Sapona) at an approximately 45 degree angle from the slide surface to gently blow the droplet from one side of the cente of the slide to the other. It is then blown off the slide. Lambda DNA is retained on aminosiline coated slides compared to an uncoated slide.

Determining Optimal Spreading Concentration for Random Location Arrays

For example a mix of an oligonucletide complementary to the sticky ends of Lambda DNA (see below) each bearing a fluorescent label are pipetted at a concentration of 0.5 uM each in 50% DMSO onto APTES coated slides. Antifade and a coverslip is added and the slide is analysed to see if individual molecules are resolvable. If not then a dilution is done e.g. 4 fold and then the solution pipetted onto the slide again and so on.

Preparation of Single Molecule Chemical Arrays

Each chemical compound in the library to be tested is synthesised with a common thiol functional group that enables covalent attachment to the slide surface. The compounds are spotted or spread, in DMF, onto maleimide-derivatized glass microscope slides. Following spotting/spreading, the slides are incubated at room temperature for 12 h and then immersed in a solution of 2-mercaptoethanol/DMF (1:99) to block remaining maleimide functionalities. The slides are subsequently washed for 1 h each with DMF, THF, and iPrOH, followed by a 1 h aqueous wash with MBST (50 mM MES, 100 mM NaCl, 0.1% Tween20®, pH 6.0). Slides are rinsed with double-distilled water and dried by centrifugation. A dilution series is done to establish optimal concentrations for single molecule detectiom. The compound to be tested may further be linked to a fluorescently detectable moiety, such as Cy3 dye.

Preparation of Single Molecule Protein Arrays

Antibody/antigen pairs provided by BD Transduction Laboratories (Cincinnati, Ohio), Research Genetics (Huntsville, Ala.), and Sigma Chemical. Antibodies are chosen which are in glycerol-free, phosphate-buffered saline (PBS) solution (137 mM NaCl, 2.7 mM KCl, 4.3 mM Na₂HPO₄, 1.4 mM KH₂PO₄, pH 7.4). Antibody and antigen solutions are prepared at a concentation chosen from range from 0.0025-0.0075 mg/ml in 384-well plates, using approximately 4 μl per well (a wider range can be first tested depending on method to be used for analysis and the spotter that is to be used. The protein solutions in an ordered array onto poly-L-lysine coated microscope slides at a 375 μm spacing using 16 steel tips or the capillary tips of the Amersham Generation m spotter. The coated slides are purchased from CEL Associates (Houston, Tex.) or are prepared as follows. Briefly, glass microscope slides are cleaned in 2.5 M NaOH for 2 h, rinsed thoroughly in ultra-pure H₂O, soaked for 1 hour in a 3% poly-L-lysine solution in PBS, rinsed in ultra-pure H₂O, spun dry, and further dried for 1 h at 80° C. in a vacuum oven. The resulting microarrays are sealed in a slide box and stored at 4° C. The arrays are rinsed briefly in a 3% non-fat milk/PBS/0.1% Tween-20 solution to remove unbound protein. They are transferred immediately to a 3% non-fat milk/PBS/0.02% sodium azide blocking solution and allowed to sit overnight at 4° C. (The milk solution is first spun for 10 min at 10,000×g to remove particulate matter). Excess milk is removed in three room temperature PBS washes of 1 min each, and the arrays are kept in the final wash until application of the probe solution (see below).

Preparing Single Molecule mRNA-Polypeptide Fusion Arrays

This is a method for linking molecular genotype with molecular phenotype. mRNA are ligated to a sequence containing a 5′ phosphate and a 3′ Puromycin.The ligated products are in vitro translatedin rabbit reticulocyte lysate kit (Ambion) for 30 minutes. The solution is adjusted to 150 mM MagCl₂ and 425 mM KCL to promote the formation of a puromycin-peptide bond. The mRNA-polypeptide fusion s are isolated by chromotagraphy. The fusions can then be arrayed onto a surface by any of the methods described in this document. For example the mRNA portion would be able to bind ot APTES surfaces by electrostatic interaction. Alternatively the mRNA could be captured by interaction with probes arrayed on enhanced aminosilane surface (Asper Biotech, Estonia). This surface would enable the protein to be better functionally active.

Making Single Molecule Arrays by In Situ Parallel Synthesis

The glass substrate can be cleaned (and all reagents used in the following steps should be of high purity) and then modified to allow ON synthesis: For epoxy derivatisation the following steps are taken. Prepare a mixture of 3-Glycidoxypropyl trimethoxysilane (98%) (Aldrich), di-isopropylethylsmine, and xylene(17.8:1:69, by volume) in a glass cylinder. Place the glass substrate in the mixture so that it is completely immersed and incubate at 80 C for 9 hours. Remove the glass from the mixture and allow them to cool to room temperature and wash with ethanol and ether by squirting liquid from a wash bottle. For adding a spacer: Incubate the glass substrates in hexaethylene glycol (neat) containing a catalytic amount of sulphuric acid (approx. 25 ul per litre) at 80 C for 10 hours with stirring. Remove the glass substrates, allow them to cool to room temperature and wash with ethanol and ether. Air Dry the plates and store at −20 C.

The array of ONs complementary to for example, yeast tRNA^{phe} is created by coupling nucleotide residues in the order in which they occur in the complement of the target sequence using a reaction cell pressed against the surface of a glass plate/slide (Knittel Glazer, Germany) which is modified (see above).

The fluidics from an ABI 394 DNS synthesizer is coupled into the reaction cell through inlet and outlet ports (instead of coupling to cpg colums). The DNA synthesizer is programmed with the following cycle (for a diamond-shaped reaction chamber with 30 mm digonal and 0.73 mm depth):

TABLE 1
Program for ABI394 DNA/RNA synthesizer to
deliver reagents for one coupling cycle.

| Step number | Function Number | Function Name | Step time (s) |
|---|---|---|---|
| 1 | 106 | begin | |
| 2 | 103 | wait | 999 |
| 3 | 64 | 18 to waste | 5 |
| 4 | 42 | 18 to column | 25 |
| 5 | 2 | reverse flush | 8 |
| 6 | 1 | block flush | 5 |
| 7 | 101 | phos prep | 3 |
| 8 | 111 | block vent | 2 |
| 9 | 58 | tet to waste | 1.7 |
| 10 | 34 | tet to column | 1 |
| 11 | 33 | B+ tet to column | 3 |
| 12 | 34 | tet to colum | 1 |
| 13 | 33 | B + tet to column | 3 |
| 14 | 34 | tet to column | 1 |
| 15 | 33 | B + tet to column | 3 |
| 16 | 34 | tet to column | 1 |
| 17 | 103 | wait | 75/140/ |
| 18 | 64 | 18 to waste | 5 |
| 19 | 2 | reverse flush | 10 |
| 20 | 1 | block flush | 5 |
| 21 | 42 | 18 to column | 15 |
| 22 | 2 | reverse flush | 10 |
| 23 | 63 | 15 to waste | 5 |
| 24 | 41 | 15 to column | 15 |
| 25 | 64 | 18 to waste | 5 |
| 26 | 1 | block flush | 5 |
| 27 | 103 | wait | 20 |
| 28 | 2 | reverse flush | 10 |
| 29 | 1 | block flush | 5 |
| 30 | 64 | 18 to waste | 5 |
| 31 | 42 | 18 to column | 15 |
| 32 | 2 | reverse flush | 9 |
| 33 | 42 | 18 to column | 15 |
| 34 | 2 | reverse flush | 9 |
| 35 | 42 | 18 to column | 15 |
| 36 | 2 | reverse flush | 9 |
| 37 | 42 | 18 to column | 15 |
| 38 | 2 | reverse flush | 9 |
| 39 | 1 | block flush | 3 |
| 40 | 62 | 14 to waste | 5 |
| 41 | 40 | 14 to column | 30 |
| 42 | 103 | wait | 20 |
| 43 | 1 | block flush | 5 |
| 44 | 64 | 18 to waste | 5 |
| 45 | 42 | 18 to column | 25 |
| 46 | 2 | reverse flush | 9 |
| 47 | 1 | block flush | 3 |
| 48 | 107 | end | |

An interrupt is set at step 1 of the next base to allow the operator (or automated x-y stage) to move the substrate one increment and restart the program. A long wait step at the beginning of the program is optional and is introduced if the operator does not wish to use the interrupt step. The operator is also advised to consult the user's manual for the DNA synthesizer. The operator is also advised to ensure there are enough reagents in the reagent bottles to last the run and to check the run of fluids through the base lines (e.g the G line may need to be continuosly flushed with acetonitrile for several minutes to ensure clear flow through).

The movement can be achieved by attaching the substrate on a High Precision TST series X-Y translation stage (Newport) and the sealing of the reaction cell is controlled in the X axis a with stepometric stage (Newport) attached with a load cell. These devices can be controlled by software created in Labview (National Instruments) on a IBM compatible personal computer.

After each base coupling, the synthesis is interrupted the plate is moved along by a fixed increment. The array can be made using “reverse synthons”, i.e. 5′ phosphoramidites, protected at the 3′ hydroxyl, leaving 5′-ends of the ON tethered to the glass. The first base is then added at the right-most position. The diameter of the reaction cell is 30 mm and the offset at each step to the left is 2.5 mm. The result is that after 12 steps, an ON complementary to bases 1-12 of the tRNAp^{he} has been synthesised in a patch 2.5 mm wide, 11×2.5=27.5 mm from the right of the plate, where the 12 footprints of the reaction cell all overlapped. At this point the footprint of the reaction cell passes on and adds the 13^{th} base, so that the next patch contains the 12-mer corresponding to bases 2-13. The process continues until, in this example all 76 bases of the tRNAp^{he} are represented along the centre of the plate. Depending on the shape of the reaction cell, in addition, the following oligonucleotidemers are also present on the array: all 11-mers are in the cells flanking the 12-mers, the next row of cells contains 10-mers and so on to the edge rows which contained the 76 mononucleotides complementary to the sequence of the tRNAp^{he}. For functionalisation the protecting groups on the exocyclic amines of the bases must be removed by Ammonia treatment. In addition this process strips oligonucleotides from the surface of the array and a long enough incubation reduces the density of probes to the level that single molecules can be individually resolved. To reduce the high density array to single molecule arrays, place the glass substrate, array side up, into a chamber that can be very tightly sealed. Add 30% high Ammonia into the chamber to cover the slides. Tightly seal the chamber and place in a water bath at 65 C for 24 hours or at 55C for 4 days. The temperature and incubation period can be adjusted depending on the density of molecules that is required (which would be defined by method for detection e.g far field or near-field). Cool before opening chamber. The array can be rinsed with milliQ water and is ready for use in hybridisation or ligation experiments (after enzymatic phosphorylation) if standard amidites are used. If as in this example, reverse synthons are used then the array can be used for hybridisaton, ligation or primer extension.

As an alternative to the destructive ammonia method, the first base coupling in the array can be mixed with monomer amidite containing a blocking group such as the base-labile protecting group 9-fluorenylmethoxycarbonyl (Fmoc) in 1: 1000 ratio (it is preferable to first optimise this by coupling patches on the same surface with different ratios of mixtures to determine optimal molecule separation for each kind of single molecule setection experiment). As this base is not labile to acid which is used to remove the dimethoxytrity protecting group in the standard chemistry, it will not get removed and therefore will not allow any further chain extension. If the Fmoc amididte is in excess it will limit the number of chains that can be synthesised. If desired the Fmoc group can be deprotected at the end of chain synthesis and functionalised with for example a group carrying a negative charge. This will help repel any non specific binding of nucleic acids and their monomers.

An in situ DNA synthesizer, geniom one (Febit, Mannheim, Germany) is commercially available. DNA synthesis on this machine can be modified to make single molecule arrays. Alternatively, once the arrays are made the channels can be flushed with destructive ammonia treatment.

Methods have also been described for preparing arrays of peptides by spatially addressable synthesis.

Further Steps in the Preparation of Arrays

Functionalising Arrays

Molecules of an arrays may be at too high a density to be individually resolvable but then the array may be functionalised so that the molecules that are detected are far enough apart to be individually resolved. This can be done as described above by destructive ammonia treatment. Alternatively, only a fraction of the molecules, each far enough from the other to be individually resolvable may be labelled and it is only these that are detected. This fractional labelling can be determined by the analyte molecules which may be labelled and are of such a concentration that they bind to the array sparsely so that despite the array molecules being closer than the minimal distance apart to be individually resolvable their interaction with the labelled analyte molecules functionalises such a fraction of the molecules such that they are far enough apart to be individually resolved.

For example as in the example given above in which olionucleotides complementary to Lambda DNA are spread on a surface at a concentration of 0.5 uM, Lambda DNA at a concentration of 10 ug/ml is found to hybridise at a density that enables each individual Lambda molecule to be individually resolved, even as the probe molecules themselves are too close to be individually resolvable by standard optical techniques.

Making Double Stranded Arrays

Any of the primary arrays of this invention that are single stranded can be made double stranded. A pool of all sequences of target length can be hybridised to the array (Buffer: 3.5 M TMACL at room temperature for 17 mers) to make it double stranded. Alternatively a common sequence is included on all molecules of the array such that a primer binds and initiates synthesis of a complementary strand.

Making Array Copies

Once a double strand array has been made as described above, the strand that is not linked to the surface, can be denatured using hot 0.1 M Alkali Buffer and then transferred to another surface to make a complementary copy array.

Secondary Arrays

Secondary arrays can be made where further molecules bind to a primary array. The further molecules are the functional molecules of the array. Molecules may be viewed as a single point source of fluorescence. Alternatively the molecules may be horizontalised and visualized as polymers. The capture process not only enables a homogenous and reproducible spread of molecules on a surface, it can also enrich molecular species of interest according to sequence. For example, all molecules of the array may include a sequence complementary to a sequence motif present in a particular gene family or may target telomeres using probes complementary to short repetitive sequences, e.g, TTAGAGAG in humans, found therein.

Target Capture

If a repertoire of single stranded oligonucleotide probes are arrayed or spread out onto a surface they can serve as capture probes either to target molecules bearing sticky ends (to which they may become ligated) or by sequence-specifically binding along a target single or double-stranded molecule under appropriate conditions.

An array of “sticky” probes can be created by designing and purchasing customized oligonucleotides (e.g drom MWG Biotech). Firstly, a binary oligonucleotide repertoire, A is created which partially contains a fixed sequence and partly contains a randomized sequence. A second oligonucleotide is provided, B which binds by complementary base pairing to only the fixed sequence on oligonucleotides of the repertoire, A. This process may be carried out entirely in solution and then the complex spread out on the surface. Alternatively, one of A or B is first spread out on the surface and then the other is reacted with it. Both the above procedures are done under conditions that enable annealing/hybridisation, for example in 4×SSC 0.2%Sarkosyl or 3.5M TMA at a temperature determined by Tm. The binding of oligonucleotide pool A with oligonucleotide B creates a repertoire of cohesive or sticky ends. These sticky ends are able to bind the termini of DNA molecules.

In another approach, the second part of the binary oligonucleotide does not comprise a repertoire of sequences but instead contains a single sequence that is complementary to a restriction digested sticky end. These sticky ends can capture complementary sticky ends of DNA digested with the appropriate restriction endonuclease. Hence, sample genomic DNA is digested with for example Not 1 Restriction endonuclease, generating sticky ends which then interact with the array capture probes.

Once a sticky end interaction has occured, a ligation reaction can be performed to covalently immobilse the target to the probes which are firmly attached to the surface. For ligation to occur between 5′ and 3′ termini, the desired 5′ termini must bear a terminal phosphate group or should be phosphorylated enzymatically using T4 Polynucleotide Kinase (New England Biolabs) as described by vendor.

The target may first be hybridised to the array in 4×SSC/Sarkosyl, unbound material removed or diluted and then ligation performed. Alternatively, the target can be directly ligated with no prior hybridisation and washing/dilution step.

Where the array comprises single stranded probes and the sticky end is provided by the target, only one strand of the target becomes covalently linked to the surface probe. Hence the non-covalently linked strand can be denatured e.g. by heating or by Alkali treatment, leaving a single stranded secondary array.

Where the array has comprised sticky probes binding to sticky ends in the target then if the 5′ termini of both sticky ends bear a phosphate group then both strands of the target duplex become immobilised. If desired one strand can be removed by addition of an exonuclease that degrades 5′ free termini or an exonuclease that degrades 3′ free termini, depending on which strand is desired to be retained on the array. One set of termini is protected from degradation due to their attachment to the surface.

It may be desirable in some instances to enable only one covalent link between sticky ends of the target and the sticky probes. In this case it is ensured that one set of sticky ends does not contain a phosphate group on the 5′ termini (phosphate groups can be removed by treatment with Shrimp Alkaline phosphatase (New England Biolabs) according to vendor suggested protocol). Such a structure enables complementary strand synthesis by Nick translation, for example. The array sticky probes can also be designed in a way that there is not a flush fit between the sticky partners in that there is a gap. left between one strand of the target and one strand of the sticky probes so that a ligation reaction cannot occur between them.

It is desirable to dephosphorylate the Notl digested DNA (as described above) to prevent self-ligation prior to ligation to the array

When the array is composed of sticky probes and binds to a single stranded target or a double stranded target which is recessed at the end, then only one target strand termini becomes covalently attached by ligation.

If single stranded DNA must be captured then measures need to be taken to make single stranded DNA e.g. by cloning the genomic library of fragments into single stranded M13 vector (see Sambrook et al) or by other means described elsewhere in this document. When the target molecule is captured and is or is made single stranded, various assays including sequence determination can be carried out on the single stranded molecules. Where sticky probes have been used, the synthesis of a complementary strand can be primed by an oligonucleotide of the sticky probe. This synthesis may be by contiguous ligation of an oligonucleotide sequence, for example, in order to assay repetitive sequences or it may be by contiguous ligation from a repertoire of oligonucleotides for DNA sequencing procedures described in this document. The sticky probes ensure that as the new strand is synthesised both it and the template remain in the same vicinity irrespective of whether harsh treatments that may denature hydrogen bonds, are performed. If this was not the case certain harsh treatments may delocalise one strand from the other and undermine the continuity of sequence acquisition.

A typical ligation reaction on surface is described by Gunderson et al (Genome Res 8 1142-53, 1988) and Pritchard and Southern (Nucleic Acid Research 25:3483) have described ligation reactions using Tth DNA ligase (Epicentre).

The addition of 10 mM MgCl₂ facillitates target capture.

Capture and Combing of Long DNA Polymers

After the above capture reactions, with or without ligation the target molecules can be horizontalised on the surface.

Capture of Sticky Ends and Horizontalisation

Linear Lambda DNA has complementary 12 base overhangs at each end which can anneal to circularise the DNA. The following oligonucleotides complementary to each end overhang are used in the following examples:

| | |
|---|---|
| Lambda A: | 5′ GGG CGG CGA CCT 3′ |
| Lambda B: | 5′ AGG TCG CCG CCC 3′. |

Surface immobilised probes capture a target and the target can become stretched out on a surface. Capture probes for lambda DNA sequence Lambda A and Lambda B, complementary to each of sticky ends of linear lambda were spotted in microarrays or spread on a surface. Spots containing completely unmatched sequences were included in the microarray. One set of A and B oligonucleotides were modified with amine and two further A and B oligonucleotides were modified with biotin. Amersham UV Crosslinking reagent (containing DMSO) was spotted with an equal volume of oligonucleotide dissolved in milliQ H₂0 was used to spot these probes onto an aminosilane modified slide (Asper, Estonia). After spotting, the slides were crosslinked at 300 mJoules followed by two washes in hot water S followed immediately by drying by blowing with forced air from a pressurised airduster canister. The oligonucleotides were spotted at 5 uM and 500 nM concentrations (using spot diameter setting 255 microns, spots per dip: 72, 55% humidity on the Amersham Pharmacia GenerationIII spotter). Lambda DNA (20 ul; 40 ug/ml was incubated with 3 ul YOYO (neat) (Molecular Probes, Oregan). The Solution was then brought up to 1 millilitre in 4×SSC 0.2%Sarkosyl. 250 ul of this was added to the Amersham Slide Processor (ASP) for a 12 hour hybridization protocol (see ASP protocol B, below). The cycle included a series of stringency washes, isopropanol flow and air drying. The flowing of the solutions and the air drying contribute to the horizontalisation and straigtening out of the DNA.

An alternative for horizontalisig DNA is manual flushing with wash reagents and isopropanol or methanol, with the slide in a vertical position. This can be done in a “Sequenza” coverplate appparatus used for immunostaining (Shandon, USA). Alternatively, the slide can be held at a 60 degree angle from the horizontal and solutions can be washed over, ensuring the solution covers all the slide.

The slide was analysed by epi-fluorescence microscope by pipetting 30 ul Fluoromount G under a coverslip and viewing on an upright epi-fluorescence microscope (Olympus BX51) fitted with a Sensys CCD camera and MetaMorph imaging software (Universal Imaging Corporation). 10× Objective was used for wide field viewing and 60× and 100×1.3 NA oil immersion lenses were used to view micorarray spots. DNA fibres were clearly visible. Better images of DNA fibres were obtained after removing the coverslip in PBS/Tween, staining with YOYO, washing with PBS/Tweeen and adding Fluoromount G.

Lambda DNA becomes immobilised and combed to spots containing sequence A and not to non-matched sequences. Mismatch probes bind with lower yield. It is also found that oligonucleotides that are complementary to double stranded regions of Lambda do not capture the lambda DNA efficiently. However, the efficiency is improved upon addition of helper oligonucelotides which bind elsewhere along the duplex to facilitate binding of internal probes.

Molecules other than linear Lambda can be horizontalised and straightened in this way by for example, sticky ends can be generated in human genomic DNA with the infrequent base cutter Not1 (as already described) which produces fragment so of an average 65 KB length which is close to the 50 kB length of Lambda DNA. Human genomic DNA fragmented in this or any other way can be spread on a surface to a produce a spatially random human genomic array. Prior to Not 1 digestion repetitive sequences can be substantially removed by the methods described elsewhere in this document. Alternatively, after immobilisation, where the DNA is single stranded repetitive DNA can be suppressed by hybridisation of unlabelled Cot-1 DNA.

Sticky ends can also be generated for capture by using the restriction endonuclease TSPR1 (NEB), according to vendor protocol, using vendor supplied buffer. This generates 9 base overhangs. The recognition sequence is redundant at a number of positions. A spatially addressable array can be made covering this sequence space. Hybridisation of TSPR1 digested genomic DNA will enable genomic DNA to be sorted according to the redundant sequences in the TSPR1 recognition sequence.

Capturing Sites in Double Stranded Regions

To enable capture at internal sites in DNA one of the following procedures can be used: List A
1) Locked Nucleic Acids (LNA) are able to form high stability interactions with nucleic acid targets. Custom sequences can be ordered from Eurogentec (Belgium). Software tools for prediction of LNA Tms are available at www.LNA-tm.com. The target can be partially denatured by high stringency conditions known in the art such as elevated temperature, 100 mM NaCl. Under these conditions LNA is able to bind the target DNA but where a normal DNA probe would likely be re-displaced by target renaturation. LNA is able to compete more effectively with renaturation of the target duplex.
2) Peptide Nucleic Acids (PNAs) which have neutral backbones are able to react with DNA under very low salt concentrationss. The target can be partially denatured by high stringency conditions known in the art such as elevated temperature, 0-100 mM cation. Under these conditions PNA is able to bind the target DNA but where a normal DNA probe would likely be re-displaced by target renaturation PNA ia able to compete more effectively with renaturation of the target duplex. PNA Tools for design of PNA probes (including PNA molecular beacons) are available at www.bostonprobes.com. Also see (Kuhn et al J Am Chem Soc. 2002 Feb. 13;124(6):1097-103) for design of PNA probes. Orum, H.; Nielsen, P.; Jorgensen, M.; Larsson, C.; Stanley, C.; Koch, T. Biotechniques 1995, 19, 472480
3) Enzymatic reactions. The ligation and polymerase reactions described in this invention aid in binding to targets, by capturing and stabilising transient interactions.
4) Padlock Probes. Padlock probes are DNA sequences in which probes are arranged in such a way that they bind to the target in way that leads to ligation around the target template in a way that they become topologically locked to the target This reaction can be done at high temperature, enabling the padlock probe to react with the target. Because it is locked to the target it effectively, cannot be displaced by renaturation of the target. The Padlock probe may contain biotin linkages which can be used for their labelling.
5) Helper Molecules
   Helper Molecules are prepared by digesting the target DNA and then adding this to non digested target DNA and renaturing and then allowing brief annealing and optional snap-cooling. This generates full length molecules in which internal regions are looped out due to the binding of the digested fragments. The looped out regions are single stranded and hence able to interact with the array probes. Alternatvely, the helper oligonucleotides may be PNA sequences complementary to the array oligonucleotide (forming a P-D loop). An RNA Helper molecule can also be used under appropriate conditions (Formamide/SSC).
6) Long Capture Probes. The capture probe may be a long molecule and thereby able to effectively compete with renaturing of the target DNA. For example, capture probes of up to around 100 nucleotides in length can be synthesised (Oswel,UK, Xeotron, USA).
7) RecA:
   Double-stranded DNA(this method is not applicable when the target is single stranded) can be probed by the the RecA mediated reaction. Aizawa and Co-workers as well as others have probed non-denatured ds DNA by using the RecA mediated strand invasion reaction. Essentially, this published protocol [Seong G H, Niimi T, Yanagida Y, Kobatake E, Aizawa Anal Chem 2000 Mar. 15;72(6):1288-93] can be followed with little modification.
      Capture of Single Stranded Nucleic Acids

Genomic DNA comes in a double stranded form and steps have to be taken to make it single stranded. Denaturation can be done by for example, putting the DNA in a boiling water bath and or raising the pH by adding for example NaOH or other alkali treatment(this may also fragment the DNA which may be desirable). However, in this case renaturation will compete with the desired target-probe interaction. When single stranded nucleic acids are obtained problematic because they can form internal base pairings (secondary structure) which compete with the target-probe interactions. Hence some of the approaches described above for capturing internal sites in double standed DNA (List A) are useful for capturing sites in ssDNA as well

Making Single Stranded DNA/RNA

One method for probing when secondary array is made with single stranded DNA.

Single strand are made e.g. by Asymmetric (long Range) PCR, magnetic bead methods, selective protection of one strand form exonuclease degradation or by in vitro RNA transcription.

Alternatively one strand can be degraded for example T7 gene 6 is able to degraded the from one of the DNA termini but not the other. As one 5′ end is attached to surface it is protected from degradation enabling asymmetric degradation. After a certain length of degradation sequence can be carried out on the exposed single strand.

Single stranded DNA can be hybridised to the array, in 4×SSC/0.2% Sarkosyl buffer at room temperature for 25 mers which may be facillitated by enzymatic reactions such as ligation or by a coaxially stacking oligonucleotide or stacking of several contiguous oligonucleotides. Sites that are known to remain accessible to probing under low stringency conditions are preferably chosen for probing (these can be selected on oligonucleotide arrays; see Milner et al, Nat Biotechnol. 1997 June;15(6):53741.).

After hybridisation the single strand is covalently attached at site of capture and then washed stringently to remove secondary structure.

The captured single stranded target can then be stretched out as described by Woolley and Kelly (Nanoletters 2001 1: 345-348) by moving a droplet of fluid across a positively charged surface.

If necessary, the density of positive charge on the surface can be controlled by coating with 1 ppm poly-L-lysine. The appropriate concentrations of other surface coatings e.g Aminoslinae need to be determined empirically.

ssDNA can be maintained at low ionic strength using 10 mM Tris, 1, M EDTA pH8 (TE bufer).

Move droplet of fluid across the surface at a velocity of Approx. 0.5 mm/s (within range 0.2-1 mm/s). This can be done by fixing the slide/mica onto a TST series translation stage (Newport), placing a droplet of fluid onto this, and translating the fluid with respect to the surface by dipping a stationary glass pipette onto the droplet. The glass pipette attracts the droplet by capillary action and the droplet remains stationary as the slide/mica is moved. After solution evaporates, rinse the mica with water and dry with compressed air (Michalet et al) Dynamic molecular combing procedure as described or the ASP procedure described above can also be used. Optionally the single stranded DNA can be coated with single strand binding protein (Amersham). Single stranded DNA can be labelled by Acridine dye or Sybr Gold (Molecualr Probes). Stretched out single stranded molecule can be probed with single stranded DNA by hybridisaton at 5 degrees C below the Tm of the oligonucleotide probe. It is preferable to use LNA oligonucleotides or PNA at 0 or up to 100 mmM NaCl. The salt concentration is kept low to minirmise intrastrand base pairing

Capturing of mRNA

mRNA bearing a PolyA tail can be captured and enriched from other nucleic acids by using oligo d(T) capture probes.

Using Arrays

Target Preparation.

Remove Cot 1 fraction as described and/or add Cot 1 DNA to the DNA to reaction mix Digest genome with Not1 restriction enzyme (NEB) as recomended by supplier.

Separate by affinity capture with a biotinylated probe (preferably LNA) complementary to sticky end generated by Not1 on a magnetic bead. Alternatively fragments are obtained using DNAse1. Altenatively target preparation is by the Random Primer labelling protocol given above with the reaction optimised to give long fragments.

Digestion may be with other restriction enzyme. For example EcoR1 which would produce shorter DNA fragments.

Alternatively fragments are obtained using DNAse1. Alternatively, target preparation can be by the Random Primer labelling protocol given elsewhere in this document with the reaction optimised to give long fragments.

If single stranded DNA is to be captured then measures need to be taken to make single stranded DNA e.g by cloning the genomic library of fragments into single stranded M13 vector (see Maniatis) or by other means described elsewhere in this document After any of the above procedures when the target molecule is captured and made single stranded, various assays including sequence determination can be carried out on the single stranded molecules. Where sticky probes have been used, the synthesis of a complementary strand can be primed by an oligonucleotide of the sticky probe. This synthesis may be by contiguous ligation of an oligonucleotide sequence, for example, in order to assay repetitive sequences or it may be by contiguous ligation from a repertoire of oligonucleotides for DNA sequencing procedures described in this document. The sticky probes ensure that as the new strand is synthesised both it and the template remain in the same vicinity irrespective of whether harsh treatments that may denature hydrogen bonds, are performed. If this was not the case certain harsh treatments may delocalise one strand from the other and undermine the continuity of sequence acquisition.

A typical ligation reaction on surface is described by Gunderson et al (Genome Res 8 1142-53, 1988) and Pritchard and Southern (Nucleic Acid Research 25:3483) have described ligation reactions using Tth DNA ligase (Abgene).

Hybridisation Assay on Arrayed Single Nucleic Acid Molecules

Hybridisation is a central feature of many procedures described in this invention. When the interacting sequence is short, hybridization requires different conditions than when the interacting sequences are long. For example, typically DNA in the 100s of base pairs range can be hybridized at a temperature above 65 degrees C. in a variety of buffers, containing SSC and optionally formamide. Other components known in the art (see Molecular Cloning, Sambrook et al) may also be included.

Where one of the interacting components is short, lower temperatures (less stringent) need to be used and problems of target renaturation and secondary structure formation must be taken into account.

A simple array containing the biallelic probe set for two SNP sequences of human TNF alpha promoter was tested. The array probes were designed with the polymorphic base at the centre of a 13 mer sequence. The array contained a dilution series of the biallelic probe set. One of two oligonucleotides with Cy3 label at the 5′ end, complementary to one of the two biallelic probes was hybridises to the single molecule array. Spots down the dilution series were analysed, and single molecule counting was done. Resolution of molecules at higher concentrations is possible by optimising the set up and by software for deconvolution. BSA, Caesin, other blocking solutions carrier DNA, tRNA, NTPs can be added in the hybridisation mix or a pre-hybridisation done to block non-specific binding. More detectable point source signal could be from the perfect match than the mismatch.

The addition of Mg2⁺ can facilitate hybridisation in some instances.

The Automated Slide Processor from Amersham Pharmacia was used for hybridisation. Hybridisation cycle for hybridisation of oligonucleotides to 13 mer oligonucleotides on array is given below.

Asp Hybridisation Protocol

PRIME PRIME WITH WASH 1

WAIT inject probe.

HEAT To 25 degrees

MIX Hybridisation mixing for 2-12 hrs

FLUSH Wash 1 (1×SSC/0.2%SDS)

HEAT To 30 degrees C

MIX Wash 1 Sminutes

PRIME Prime with wash 2(0.1×SSC/0.2%SDS)

FLUSH Wash 2

MIX Wash 2 30 seconds

FLUSH Wash 3 (Wash (0.1×SSC)

MIX Wash 3 30 seconds

PRIME Prime with was 4(0.1×SSC)

FLUSH Wash 4 (0.1×SSC)

Prime Prime with Isopropanol

Plush Flush with Isopropanol

Flush Flush with air

Airpump Dry Slide

Heat Turn off Heat

Alternatively, a manual hybridization set up as known in the art can be used. Briefly, a droplet of hybridization mix is sandwiched between the array substrate and a coverslip. The hybridization is performed in a humid chamber (edges are optionally sealed with nail polish).

The coverslip is slid off in wash buffer and washes are done preferably with some shaking.

The results are analysed by TIRF microscopy using oxygen scavenging anti-fade solution.

In Situ Denaturation of Horizontalised DNA Followed by Probing

Once a molecule is horizontalised, for many applications, it needs to be made further available for interaction with oligonucleotide probes.

When DNA is arrayed or captured on the surface the following protocol (based on Zhong et al PNAS 2001 Mar. 27;98(7):3940-5.) can be used to probe regions:

Approximately 200 ng of each probe in 20 μl hybridization mixture (50% formamide, 10% dextran sulfate, 2×˜SSC, 100 ng/μl salmon sperm DNA, and 100 ng/μl human Cot-1 DNA) was denatured by boiling for 5 min. Arrayed horizontalised DNA is denatured by incubation in 70% formamide, 2×SSC at 70° C. for 2 min, and dehydrated through ice-cold ethanol series (70%, 90%, and 100%) 3 min each and air-dried. The hybridization mixture is applied to the arrayed horizontalised DNA and incubated overnight at 37° C. The slide is washed three times for 5 min 2×˜SSC at 37°.

Alternatively the above protocol can be carried out using 6.2M Urea instead of the Formamide as denaturant (based on Castro and Williams, 1997, Anal. Chem. 69:3915-3920).

The advantage of these type of protocols is that although the DNA becomes denatured, the single strands are not able to re-nature or form secondary structure due to the interactions that are made with the surface.

It is preferable to apply one of the approaches provided in List A.

The probes may be linked with labels such as 20 nM Fluosphere nanoparticles before binding to arrayed DNA or alternatively they may be biotinylated and and streptavidin linked Semiconductor Nanoparticles can bind to them before or after the DNA is arrayed on the surface, 45 degrees C for 1 hour in Quantum Dot buffer is sufficient for this. The nanoparticles can be reacted with 1 mg/ml BSA or caesin or other appropriate blocking mix solution to avoid non-specific absorption onto the glass surface.

DNA can be array captured and probed as illustrated: Dephosphorylate Lambda DNA (500 ug/ul) with calf alkaline phosphatase (this step minimizes concatemerization and circularization of Lambda DNA). Hybridise lambda to array containing complementary probes to its sticky end, using ASP Protocol B. Optionally treat slide with BSA or Caesin or other blocking solution. Add probes and label e.g. semi-conductor nanoscrystals (Molecular Probes, Oregon) in buffer provided by vendor Wash in PBS/Tween followed by PBS wash. Visualize DNA and fluorescent nanoparticles captured and horizontalised on the array.

Probing Followed by Horizontalisation

The target DNA can be partially denatured in solution, then probes in solution are able to bind to or invade sites in DNA, particularly AT rich regions. LNA oligonucleotides can bind partially denatured ds DNA in solution at temperatures for example ranging from around d45 degrees C. to around 95 degrees C. depending on sequences and lengths Salt concentrations higher than 100 mM can be used, eg, 3×SSC or 4×SSC. In as similar way PNA probes are abel to hybridise although little or no salt is required (eg 40 mM NaCl or 6.2 M Urea). Once LNA or PNA probes are bound they are able to persist on the DNA to a greater extent than DNA probes. Alternatively, Padlock probes can be reacted onto the DNA. These become permanently fixed. Following binding of the probes the DNA can be combed by the nethods of this invention. The probes may be attached to labels such as 20 nm Fluospheres. Alternatively they may be biotinylated and streptavidin linked Semiconductor Nanoparticles can bind to them before or after the DNA is arrayed on the surface, 45 degrees C. for 1 hour in Quantum Dot buffer is sufficient for this.

Probing of Concatemerized Lambda DNA

Concatemerize Lambda DNA by mixing 2 ul Lambda DNA(500 ug/ml) with 1 ul Thermal T4 RNA ligase (Epicentre), 8 ul 5×Ligase Buffer (supplied with enzyme). Incubate at 65 degrees C. for 30 minutes. Then add 8 ul of biotinylated Lamda sequence A and B and Streptavidin coated Fluosphere mix to the Ligation reaction. Incubate for a further 30 minutes at 65 degrees C. Incubate with YOYO for at least 20 minutes. Horizontalise the DNA onto an untreated glass slide or dilute and incubate on a aminosilane coated slide. Dry slide and mount with Fluoromount G. Horizontalisation/straightening can be done by one of a number of different methods described in this document. Upon visualization on an epi-fluorescence microscopy a recurring sequence on the lambda concatamers is labelled by Fluorosphere complex (see FIG. 10b).

Ligation Assay on Single Molecule Array

Target preparation is essentially as for SNP typing/resequencing section and target analysis Mix:

5×ligation buffer*

Solution oligonucleotide 5-10 pmol, labelled with fluorescent dye on 3′ and phosphoryalted on 5′ end

Thermus thermophilus DNA ligase (Tth DNA ligase) 1 U/ul,

Target sample

Add to centre of array* *

Add coverslip over the top of array area and seal edges with rubber cement

Place at 65° C. for 1 hr.
*5×ligation buffer is compose d of 100 mM Tris-HCL pH 8.3, 0.5% Triton X-100, 50 mM MgCl, 250 mM KCl, 5 mM NAD+, 50 mM DTT, 5 mM EDTA

** In this example different sequences that define the allele of a SNP are placed in adjacent spots in the microarray, by the spotting methods described. The last base of these sequences overlap the variant base in the target. The oligonucleotide on the array are spotted with 5′ aminatation. The 3′ end is free for ligation with the 5′ phosphorylated solution oligonucleotide. Alternatively the array oligonucleotide can be 3′ aminated and 5′ phosphorylated The solution oligonucleotide can be phosphorylated and labelled on the 5′ end. The solution oligonucleotide is preferably a mixture of every 9 mer (Oswel, Southampton, UK).
*5×ligation buffer is compose

Preparation of Sample DNA

From Amplicons

Produce amplicons by methods known in the art covering the desired region, ethanol precipitate and bring up in 125 ul water. Optimally the amplicons should be 100 bases or less. If they are longer than 200 base pairs then the following fragmentation protocol must be used. Fragment the amplicons as follows: To the 12.5 ul add 1.5 ul of Buffer(500 mM Tris-HCl. pH(0.0; 200 mM (NH4)2SO4) . Add 0.5U (1U/ul) of Shrimp Alkaline Phosphatase (Amersham). Add 0.5ul of thermolabile Uracil N-Glycosylase (Epicentre). Incubate at 37 for one hour and then place at 95 degrees for ten minutes. Check fragmentation on a gel (successful if no intact PCR is detected).

Genomic DNA can be extracted and purified

Digest DNA with restriction enzyme or random fragmentation(e.g. DNAs1 treatment)

Restriciton Digest:

DNA X ul for lug

Reaction 3 10×Buffer 5ul

EcoR1 2ul (20 units)

Water Y ul to a final volume of 50 ul

Incubate 37 degrees for 16hours

Stop reaction by by 72 C for 10 minutes

Purify digested DNA using a commercial purificaton kit (Zymo Research's DNA clean and Concentrator) as per supplied protocol

Cot 1 DNA can be used at this stage to remove repetitive DNA and/or can be added to array hybridisation/reactions for in situ suppression of hybridisation of probes to repetitive DNA by blocking the repetitive DNA by hybridisation to the Cot-1 DNA.

Ex situ depletion of repetitive sequence:

Cot-1 DNA (Gibco BRL) is labelled with biotin using Biotin Chem-Link kit (Boehringer Mannheim) or photoprobe Biotin Kit (Vector Laboratories) as per manufacturs protocol and purified with Sepahdex G50 Columns(Amersham Pharmacia) as per manufactureres protocol.

A 700 ng amount of source DNA is hybridised with 35 ug (50 fold excess) of biotin-labelled Cot-1 DNA.

Streptavidin magnetic particles (Boehringer Mannheim) are prepared according to manufacturers instructions, 4.4 mg to a final 125 ul volume

The Streptavidin-magnetic particles are applied to the targe-tDNA-biotin-labelled Cot1 DNA(100 ul). After incubation f the Magnetic bead captured Cot-1 fraaction was separated to the side of the tube with a magnet, and the supernatant containing the target DNA pipetted to a fresh tube. The magnetic separation is repeated, and then the target DNA supernatent is purified using a QIAex II kit (Qiagen).

In situ Blocking of Cot-1 Fraction

Add 25-125 ug (or 100 fold excess to target DNA) of Cot-1 DNA directly to hybridisation/reaction mix

Apply directly to the array.

Alternatively, the DNA can be randomly amplified by random primers using reagents for Spectral Genomics(SG) (Houston, Texas) Human BAC array and BioPrime labelling kit form Gibco/BRL.

Add SG Sterile Water(orange vial) to xul (at least 100 ng not more than 1 ug) of digested DNA to bring volume to 25 ul. Add 2.5× random primer /reaction buffer (Gibco). Mix the samples well and boil for 5 minutes and place the samples on ice for 5 minutes

On ice add 2.5 ul of SG labelling Buffer(yellow vial) toeach sample

Optionally add 1.5 ul Cy5-dCTP or Cy3-cCTP to the samples (In some sequencing embodiments, a mixture of for example Cy5-dCTP and Cy3-dATP may be added to intrinsically label the DNA strand with two labels; the 5 other combinations of dNTPs may also be required in separate reactions)

Add 1 ul Klenow Fragment (Gibco) to the sample and mix well by tapping and recollecting by centrifugation

Incubate the sample at 37 degrees from 2.5 hours (enough for one or two array hybridisations/reactions) to overnight (produces sufficient material for several array hybridisations). The probe will range in size between 100 and 500 bp.(for sequencing applications it may be desirable to have longer sequences and for this the concentration of the random primer can be diluted (the concentration of random primer to use to get a particular random primer product must be determined empirically).

Stop the reaction by adding 0.5 ul 0.5 M EDTA pH 8 and incubating at 72 for 10 minutes. Place samples on ice until use or freeze at −20.

If necessary the random prime labelled DNA can again be depleted for any sequences from the Cot-1 fraction by magnetic separation with Cot-1 DNA.

Alternatively or in addition Cot-1 DNA can be added to the hybridisation/reaction mix.

Fragmentation Methods

Fragmentation of the genome to the desired size can be done by DNAse 1 treatment timised for a prticular enzyme. Fragmentation by sonication can also be optimised to give fragments of a desired length DNA can be sheared by passing it through a narrow gauge needle. Heating and UV light exposure may also fragment DNA as appropriate for use in this invention.

Nanoparticle Bioconjugation and Purification

Oligonucleotides can be coupled to microspheres (Luminex, Austin Tex.) or nanospheres by a one step carbodiimide coupling method. Each coupling reaction contains 10.1 uM of amino-substituted oligonucleotide and 1×10⁸ microsheres/ml in 0.1 MES. PH 4.5. EDC is added at 0.5 mg/ml and reaction is incubated for 30 minutes st room temperature followed by a second EDC addition and incubation. The coupled microspheres are washed and stored at 4 degrees C. in the same buffer.

Dendrimers are coupled to oligonucleotide-microspheres in [tetramethylammonium chloride (TMA) buffer: 0.01% SDS, 50 mM Tris, 3.5 M TMA, 0.002 M EDTA or 2-6× sodium citrate (SSC) buffer: 0.9 M NaCl, 0.03 M trisodium citrate. <2×SSC gives more specificity of binding at 40 degrees C. Dendrimers can be synthesised using branched phosphoramidites (MWG Biotech, Germany)

There are two approaches for the use of streptavidin nanoparticles (Quantum Dot Corp, USA) to label probe oligonucleotides:

A Hybridise biotinylated oligonucleotide to DNA and then add streptavidin coated nanoparticle or

B Complex streptavidin-nanoparticle to biotinylated oligonucleotide and hybridise to DNA

In Method A there will be no steric hindrance by nanoparticles to hybridisation of the oligonucleotide to the target DNA. However, even though the oligonucleotide is coupled to the nanoparticle before hybridisation, in method B, it is not too different a situation to DNA binding to oligonucleotides bound to a surface in microarrays, which obviously works. Preferably the nanoparticles need to be coupled to the oligonucleotide probes in advance of hybridisation, as in method B, in a one-colour/one-allele specific way. This is so that the allele in the target can be typed by looking at which of the two colours localises by hybridisation to a particular SNP site. For method B, firstly, excess biotinylated oligonucleotide can be added to the beads so that substantially all the beads become attached with oligonucleotide (one should estimate the amount of nanoparticle and add oligonucleotide at e.g. 1000-10,000 excess) then unreacted oligonucleotide needs to be separated and discarded. This separation can be done by one of the following three methods:

1 Dialysis

2 Chromaspin columns (eg chromaspin 100).

3 Ultra-centrifugation at its highest speed setting (e.g. 120K rpm).

4 Streptavidin coated magnetic beads

3 Vectrex columns (Vector Laboratories

A nanoparticle attached oligonucleotide probe can be reacted with sample (e.g. lambda) DNA that has already been horizontalised. This can be done in the presence of BSA and/or other blockers. Alternatively the nanoparticle oligonucleotide can be reacted with lambda before combing. If this is done then, before combing, the reaction should be put through Chromospin 1000 (Clontech, USA) which can separate the long DNA target fragment from smaller products.

Nanoparticle can be reacted with 1 mg/ml BSA/Caesin solution to avoid absorption of the beads onto the glass surface.

Genomic DNA Labeling Protocol

The following protocol is developed for microarray-based comparative genomic hybridization but can also be used for other applications of this invention.

Genomic DNA can be labeled with a simple random-priming protocol based on Gibco/BRL's Bioprime DNA Labeling kit, though nick translation protocols work too. I routinely use the BioPrime labeling kit (Gibco/BRL) as a convenient and inexpensive source of random octamers, reaction buffer, and high concentration klenow (do not use the dNTP mix provided in the kit), though other sources of random primers and high concentration klenow work as well.

1. Add 2 ug DNA of the Sample to be Labeled to an Eppindorf Tube.

Note: For high complexity DNAs (e.g. human genomic DNA), the labeling reaction works more efficiently if the fragment size of the DNA is first reduced. I routinely accomplish this by restriction enzyme digestion (usually DpnII, though other 4-cutters work as well). After digestion, the DNA should be cleaned up by phenol/chloroform extraction/EtOH precipitation (Qiagen PCR purification kit also works well).

2. Add ddH₂0 or TE 8.0 to bring the total volume to 21 ul. Then add 20 ul of 2.5× random primer/reaction buffer mix. Boil 5 min, then place on ice.

2.5×X random primer/reaction buffer mix:

125 mM Tris 6.8

12.5 mM MgCl₂

25 mM 2-mercaptdethanol;

750 ug/ml random octamers

3. On ice, add 5 ul 10×dNTP mix.

10×dNTP mix:

1.2 mM each dATP, dGTP, and dTTP

0.6 mM dCTP

10 mM Tris 8.0, 1 mM EDTA

4. Add 3 ul Cy5-dCTP or Cy3-dCTP (Amersham, 1 mM stocks)

Note: Cy-dCTP and Cy-dUTP work equally well. If using Cy-dUTP, adjust 10×dNTP mix accordingly.

5. Add 1 ul Klenow Fragment.

Note: High concentration klenow (40-50 units/ul), available through NEB or Gibco/BRL (as part of the BioPrime labeling kit), produces better labeling.

6. Incubate 37 degrees C for 1 to 2 hours, then stop reaction by adding 5 ul 0.5 M EDTA pH8.0

7. As with RNA probes, I purify the DNA probe using a microcon 30 filter (Amicon/Millipore):

Add 450 ul TE 7.4 to the stopped labeling reaction.

Lay onto microcon 30 filter. Spin ˜10 min at 8000 g (10,000 rpm in microcentrifuge).

Invert and spin 1 min 8000 g to recover purified probe to new tube (˜20-40 ul volume).

8. For two-color array hybridizations, combine purified probes (Cy5 and Cy3 labeled probes) in new eppindorf tube. Then add:

30-50 ug human Cot-1 DNA (Gibco/BRL; 1 mg/ml stock; blocks hybridization to repetitive DNAs if present on array).

100 ug yeast tRNA (Gibco/BRL; make a 5 mg/ml stock; blocks non-specific DNA hybridization).

20 ug poly(dA)-poly(dT) (Sigma catalog No. P9764; make a 5 mg/ml stock; blocks hybridization to polyA tails of cDNA array elements).

450 ul TE 7.4

Concentrate with a microcon 30 filter as above (8000 g, ˜15 min, then check volume every 1 min until appropriate). Collect probe mixture in a volume of 12 ul or less.

9. Adjust volume of probe mixture to 12 ul with ddH₂0. Then add 2.55 ul 20×SSC (for a final conc.of 3.4X) and 0.45 ul 10% SDS (for a final conc. of 0.3%).

Note: The final volume of hybridization is 15 ul. This volume is appropriate for hybridization under a 22 mm2 coverslip. Volumes should be adjusted upwards accordingly for larger arrays/coverslips.

10. Denature hybridization mixture (100° C., 1.5 min), incubate for 30 minutes at 37° C. (Cot-1 preannealing step), then hybridize to the array.

11. Hybridize microarray at 65° C. overnight (16-20 hrs). Note, see Human Array Hybridization protocol for details on hybridization.

12. Wash arrays as with mRNA labeling protocol and scan:

First wash: 2×SSC, 0.03% SDS, 5 min 65° C.

Second wash: 1×SSC, 5 min RT

Third wash: 0.2×SSC, 5 min RT

Note: the first washing step should be performed at 65° C.; this appears to significantly increase the specific to non-specific hybridization signal.

Two methods for probing when secondary array is made with ds DNA are given.

The problem with denaturation and probing with a single probe for sequencing by hybridisaton when the target is double-stranded is that it is not known which of the sense or antisense strand each probe binds to. This is overcome in the double complementary probe strategy by probing both strands simultaneously.

There are two problems when trying to probe and view single molecules with oligonucleotides, along a combed molecule. One is to differentiate real signal from non-specific (to get get sufficient signal to be detected above background) and the second is to get access to the DNA sequence for binding of probe.

Specific Applications

Mini-Sequencing

The sample anneals to arrayed primers which promote DNA polymerase extension reactions using four fluorescently labeled dideoxynucleotides. In these examples both strands of the target can be analysed simultaneously. But in other cases it may be chosen to use single stranded products (eg, by asymmetric PCR, RNA transcription, selective degradation of one strand or biotinylation of target strand and removal of non-biotinylated other strand by for example, magnetic beads methods known in the art.

Wash enhanced aminosilane slides with milliQ water before using and dry (e.g place on 58 C heating plate). Denature the sample DNA for 6 minutes at 95 degrees. Centrifuge and put on ice. Add 5 ul of dye terminators (e.g Texas Red-ddATP, Cy3-ddCTP, Fluorescein-ddGTP, Cy5-ddUTP, all 50 uM) and diluted Thermosequenase (4 U/ul), mix and pipette onto slide covering region carrying the array. Immediately cover with a piece of Parafilm to cover the array area if the array has been printed on a coverslip or place Parafilm or coverslip over array if it has been printed on a slide. Lifter coverslips (Erie Scientific) are preferably used. Incubate slide 25 minutes at 58 C. Remove Parafilm/coverslip, wash slide 2 minutes in 95 degree miliQ water, 3 minutes in 0.3% Alcanox solution and 2 minutes in 95 degree milliQ water.

| | |
|---|---|
| Excitation Wavelengths | 4 lasers |
| | 488 nm (FITC) |
| | 543 nm (Cy3) |
| | 594 nm (Texas Red) |
| | 633 nm (Cy5) |
| Emission Wavelengths | 8 position filter wheel with |
| | narrow band pass filters |
| | 530 nm (FITC) |
| | 570 nm (Cy3) |
| | 630 nm (Texas Red) |
| | 670 nm (Cy5) |

A droplet of slowfade Light antifade reagent (Molecular probes) is added to minimize photobleaching and cover with a coverslip

If non-specific sticking of for example labelled nucleotides (seen by for example signals outside the regions carrying the microarray spots, then prehhybridisation of the array can be done (e.g. in a 25 ml volume in a 50 ml falcon tube) with a buffer containing 1%BSA, 0.1% SDS (and or Sarksyl) and optionally Cot1 DNA, poly(A) DNA, tRNA.

Errors are eliminated by methods of this invention, for example by an algorithm or by enzymatic methods such as the use of Apyrase. For the latter, 8 mU of Apyrase (Sigma) is added to the reaction mix on the array.

The array for this experinent can be made as in example above (with reducton of synthesis cell dimension and step size) or by spotting 5′ aminated oligonucleotides onto enhanced aminosilane slides in DMSO:Water at an appropriate dilution (eg 50-500 nM range)

Haplotyping

Probing a Horizontalised DNA Polymer at Multiple Loci Using Two-Colour Probes

Each locus of interest is probes bya biallelic probe comprising allelic probes labelled with different fluorescent tags. For example one, allele is labelled with a semiconductor nanocrystal emitting at 565 nm whilst the other one emits at 655 nm.

The target molecule may be spread with or without the aid of a capture molecule. Where a capture molecule is provided it may probe the first allele of interest. The target molecule may be captured at a second point by arrayed capture probes, which may also be allele specific. Different allele specific array capture probes would be placed at distinct spatial locations by the arraying methods described in this document and known in the art. The double capture would be done using 4×SSC/Sarkosyl at a temperature determined by the Tms of the probes. Subsequent internal probing of the captured molecule is via any of the approaches descriebd in this document. Each subsequent SNP site would be probed by specific complementary allele specific probes but as the target molecule is horizontalised, the same two labels need be used.

Directing Different Loci on a Single Polymer Molecule to Different Spatial Locations

Probes were placed at spatially distinct gold electrode pads separated by a gap of approximately 5-10 um and DNA was bridged over a gap between adjacent pads. The sticky ends of Lambda DNA was reacted with complemetary probes in 4×SSC 0.1% Sarkosyl. Similarly probes can be spaced strategically to capture other sequences along the same DNA polymer, the spatial location to which the DNA polymer binds being indicative of the sequence present at that locus on the DNA.

The intervening DNA is not substantially bound to the surface when high salt is used (if surface is APTES coated) and this makes the DNA available for probing by any of the methods mentioned.

Obtaining Sequence Information by Hybridisation

Where the sample to be sequenced are oligonucleotides then the number of different of probes that need to be hybridised may not be too large and positional information may not be required.

There are two fundamental aspects of the single molecule sequencing of this invention. Spatially address genome. Probing along the DNA polymer in a manner that information about what positional along the DNA polymer each probe binds to is obtained.

There are several schemes with which single molecule sequencing by hybridisation can be achieved. The following gives a number of strategies. Experimental steps that are common are described under separate headings. Other methods are elsewhere in the description of methods.

Sequencing Strategy Example A

Sequencing of spatially addressably captured genomic DNA is done by iterative probing with 6 mer oligonucleotides. There are 4096 unique 6 mers . Each oligonucleotide is added one after the other. The position(s) of binding of each oligonucleotide is recorded before addition of the next oligonucleotide. The target is preferentially in a linearised single stranded form.

Sequencing Strategy Example B

Sequencing of spatially addressably captured genomic DNA is done by iterative probing with sets of 6 mer oligonucleotides. There are 4096 unique 6 mers, these are split into groups of 8 containing 512 oligonucleotide each. Each probe is labelled via a C12 linker arm to a dendrimer(Shchepinov et al Nucleic Acids Res. 1999 Aug. 1;27(15):3035-41) which carries many copies of this probe sequence (this construct is made on an Expedite 8909 synthesizer or an ABI 394 DNA synthesizer or custom made by Oswel). The 512 probe constructs of each set are hybridised simultaneously to the secondary genomic array. Following this the position of binding of the probes and the identity of the probes is detected by hybridisation of a library of microspheres, within which each microsphere is coated with a complementary sequence to one of the probe sequences (e.g by first coating mucrosphere with streptavidin (Luminex) and then binding biotinylated oligonucleotides to this as described above or binding aminated oligonucleotides by carbodiimide coupling; see also Bioconjugate techniques, Greg T. Hermanson Academic Press). The arms of the dendrimer form multiple interactions with the multitude of oligonucleotide copies that coat the microsphere in <400 mM Monovalent salt, Na at 40 degrees C. or above. The microsphere in one of a coded set, ratiometrically dyed with a two or more dyes(100-1000 differnt coded beads are available (Lumonics). The spectral proprties of these beads that now decorate the DNA in the secondary array and their position of binding are recorded. The probes are then denatured which releases the whole complex. The array can then be probed with the 8 other probe sets in a stepwise manner. The probe concentrations are configured such that only some of the sites on the DNA are occupied, but analysis of the multidude of copies of each genomic fragment within a microarray spot enables information about all the sites that are occupied to be worked out. The information obtained from the experiment is fed into the sequence reconstruction algorithm. Optionally the 8 sets can be further split and hybridisation is done on multiple copies of the array. In this way far fewer coding beads need be used.

Sequencing Strategy Example C

Sequencing of spatially addressably captured genomic DNA is done by iterative probing simultaneously with sets of non overlapping or minimally-overlapping sequences added together and substantially overlapping sequences are added separatedly. Non-overlapping and minimaly overlapping sets of sequences from this set of 4096 are determined algorithmically. Each set is added one after the other. The position(s) of binding of oligonucleotidess in each set is recorded before addition of the next oligonucleotide. The target is preferentially in stretched single stranded form.

The information that is passed onto the algorithm for sequence reconstruction is the identity of the sequences in the non overlapping set, that they do not overlap, the positions of binding of probes from the set This is preferably done with a high resolution method such as AFM and the probe molecules need not be labelled. In another embodiment each probe is labelled for example, with a streptavidin molecule separated by a linker. The draft sequence of the genome is used to reconstruct the sequence.

Sequencing Strategy D

The 4096 oligonucleotides are grouped into sets, in this example in sets of sixteen each containing 256 oligonucleotides (oligonucleotides in each set are chosen by algorthm to minimally overlap in sequence). Each set is used in a series of hybridisations to a separate copy of the secondary array. After smmultaneous hybridisation of the 265 oligonucleotidenucletides in the set and recording of the position of their binding they are denatured. Next one of the oligonucleotides from the set is ommitted and the resulting set of 255 oligonucleotides is hybridised back to the array. The absence of signals from positions where there was previously signal tells us the identity of the oligonucleotide that bound in that position before as being the oligonucleotide that is ommitted in the present run. This is iterated with a different oligonucleotide from the set and so on, 256 times so that information is obtained from sets in which one of the 256 is omitted each time. The oligonucleotides are bound in saturating concentrations. The information that is obtained is passed onto the algorithm for sequence reconstruction.

Sequencing Strategy Example E

Sequencing of spatially addressably captured genomic DNA is done by iterative probing with complementary pairs of 6 mer oligonucleotides, both oligonucleotides labelled with the same label. There are 4096 unique 6 mer complementary pairs. Each pool is added to a separate S secondary array (capture probes to which the genomic sample array has been spatially addressably captured and combed). After each probing step the 6 mers are be denatured and then a different complemntary pair is added

The target is preferentially double stranded in this example and not denatured in situ. However denaturation in situ is an alternative.

Each of one the 256 BainsProbes in each pool will be hybridised to a secondary array. To reduce time and the affects of attrition on the secondary array, multiple BainsProbes are annealed at one time. In this example two will be labelled at one time and preferentially, these will be differentially labelled, for example each of the 2 can be labelled with Cy3 or Cy5 dyes or a red fluorescent or green fluorescent Fluorosphere (a more complex coding can be devised or alternatively there would be no labelling and it would be the task of the algorithm to reconstruct the sequence on that basis). After annealing, the position of the probes is recorded with respect to each other and the markers. In some embodiments the DNA probes can be denatured from the target DNA, before another set is added (or after several sets are added) but in the present example, the BainsProbes are not removed after hybridisation Instead, after recording the positions of probe binding, the next pair of probes are added This will need to be iterated 128 times to go through all the probe pairs. If each iteration is approximately 10 minutes for each addition, then the sequencing will be complete within 24 hours. This can be speeded up further if more than 2 oligonucleotides are added at a time, for example 80 oligonucleotides added at a time would allow whole genome sequencing in about an hour; each of the 80 would not need to hybridise to every copy that is captured within a microarray spot, for example if there is 2000 50 kb molecules captured in one spot, then each molecule need only be labelled with say, 8 probes. This can aid in one sequence preventing the binding of another by forming overlap with another.

Molecular beacons can be used as probes: here there is no fluorescence when the oligonucleotide is scanning the molecule, only signal when it forms a stable enough duplex to unwind the stem and release the fluorophore from quenching. Two types of molecular beacons can be used, one based on FRET and the other based on electron transfer (Atto-Tec, Heidelberg). It is likely that as sequence reconstruction in this case will utilise the draft sequence of the genome, the

Sequencing Strategy Example F

Sequencing of spatially addressably captured genomic DNA is done by iterative probing with 8 mer oligonucleotides. Each 8 mer contains 6 unique bases and two degenerate positions, in this example, the central two bases are degenerate. There is 4096 different probes identified by their 6 unique positions but each of these carry 16 different sequences due to the degenerate positions (these will be referred to as BainsProbes after Bains and Smith Journal of theoretical biology 135: 303-307 1988). The 4096 BainsProbes are split into 16 pools of 256 BainsProbes (this is an arbitary choice and they can be split into 4 pools of 1024 if the number of arrays are limiting) with each pool containing sequences approximately matched for Tm. Each pool is added to a separate secondary array (capture probes to which the genomic sample array has been spatially addressably captured and combed).

Each of one the 256 BainsProbes in each pool is hybridised to a secondary array. To reduce time and the affects of attrition on the secondary array, multiple BainsProbes are annealed at one time. In this example two are labelled at one time and preferentially, these are differentially labelled, in this example each of the 2 are labelled with either Cy3 or CyS dye or a red fluorescent or green fluorescent Fluorosphere (a more complex coding can be devised or alternatively there would be no labelling and it would be the task of the algorithm to reconstruct the sequence on that basis). After annealing, the position of the probes is recorded with respect to each other and the markers. In some embodiments the DNA probes can be denatured from the target DNA, before another set is added (or after several sets are added) but in the present example, the BainsProbes are not removed after hybridisation. Instead, after recording the positions of probe binding, the next pair of probes are added This will need to be iterated 128 times to go through all the probe pairs. If each iteration is approximately 10 minutes for each addition, then the sequencing will be complete within 24 hours. This can be speeded up further if more than 2 oligonucleotides are added at a time, for example 80 oligonucleotides added at a time would allow whole genome sequencing in about an hour; each of the 80 would not need to hybridise to every copy that is captured within a microarray spot, for example there may be 2000 50 kb molecules captured in one spot, and each individual molecule copy need only be labelled with say, 8 probes. This can aid in one sequence preventing the binding of another by forming overlap over a complementaryy region.

Molecular beacons can be used as probes: here there is no fluorescence when the oligonucleotide is scanning the molecule, only signal when it forms a stable enough duplex to unwind the stem and release the fluorophore from quenching. Two types of molecular beacons can be used, one based on FRET and the other based on electron transfer (Atto-Tec, Heidelberg). It is likely that as sequence reconstruction in this case will utilise the draft sequence of the genome, the

Sequencing Strategy Example G

Sequencing of spatially addressably captured genomic DNA is done by iterative probing with 13 mer oligonucleotides (this lenght can form stable duplex at room temperature). Each 13 mer contains 6 unique bases and 7 degenerate positions, for example, 8 bases at the 5′ end are degenerate (will be called stabiliser probes). Although we have the stability of a 13 mer we will only have the sequence infromation of a 6 mer. There will be 4096 different probes identified by their 6 unique positions but each of these will carry ca. 16,384 different sequences due to the degenerate positions. In this example the concentration of oligonucleotide will be 100 to 1000 fold higher than in example A. The 4096 Stabiliser Probes will be split into 8 pools of 512(this is an arbitary choice and they can be split into 4 pools of 256) with each pool containing sequences approximately matched for Tm. Each pool will be added to a separate secondary array (capture probes to which the genomic sample array has been spatially addressably captured and combed).

Each of one the 128 BainsProbes in each pool will be hybridised to a secondary array. To reduce time and the affects of attrition on the secondary array, multiple BainsProbes are annealed at one time. In this example two will be labelled at one time and preferentially, these will be differentially labelled, for example each of the 2 can be labelled with Cy3 or Cy5 dyes or a red fluorescent or green fluorescent Fluorosphere (a more complex coding can be devised or alternatively there would be no labelling and it would be the task of the algorithm to reconstruct the sequence on that basis). After annealing, the position of the probes is recorded with respect to each other and the markers. In some embodiments the DNA probes can be denatured from the target DNA, before another set is added (or after several sets are added) but in the present example, the BainsProbes are not removed after hybridisation. Instead, after recording the positions of probe binding, the next pair of probes are added This will need to be iterated 128 times to go through all the probe pairs. If each iteration is approximately 10 minutes for each addition, then the sequencing will be complete within 24 hours. This can be speeded up further if more than 2 oligonucleotides are added at a time, for example 80 oligonucleotides added at a time would allow whole genome sequencing in about an hour; each of the 80 would not need to hybridise to every copy that is captured within a microarray spot, for example if there is 2000 50 kb molecules captured in one spot, then each molecule need only be labelled with say, 8 probes. This can aid in one sequence preventing the binding of another by forming overlap with another.

Molecular beacons can be used as probes: here there is no fluorescence when the oligonucleotide is scanning the molecule, only signal when it forms a stable enough duplex to unwind the stem and release the fluorophore from quenching. Two types of molecular beacons can be used, one based on FRET and the other based on electron trnsfer (Atto-Tec, Heidelberg). It is likely that as sequence reconstruction in this case will utilise the draft sequence of the genome, the

The above examples are all done with 6 mer probes, however the strategies can be implemeted with oligonucleotidenucleotdes shorter than 6 nt, in which case there will be fewer cycles but more stabilising chemistries such a LNA will be used. Alternatively oligonucleotides longer than 6 nt can be used in which case there will be more cycles.

These three strategies serve as examples but methods from any of these can be adapted from one to the other and there are several other specific means which are apparent from the methods and protocols described in this invention For example, each probe can be ligated to a random library of ligation molecules, this would serve to stabilise the interactions and eliminate mismatches.

Getting Additional Experimental Validating Sequence Information

To get further information about sequence, during preparation the DNA sample can be internally labelled with combinations of base labelling fluors as suggested in the random primer labelling section above. In addition where the target DNA of the secondary array is double stranded, optical mapping in which gaps are created at the site of restriction digest can provide sequence and positional information.

Sequence Reconstruction, Re-mining and Validation

A first pass at reconstructing the sequence is attempted. This will identify regions with gaps and low confidence.

As the draft human genome sequence is known, any gaps can be filled in by probing with specific oligonucleotides targeting the gapped/low confidence region on a further array and this process can be reiterated (i.e. see if additional information allows reconstruction, if not add further probes to same array or separate array and repeat).

Sequence reconstruction can be performed on a network of desktop computers, e.g IBM compatible Personal computer, Apple personal computer, or Sun Microsystem computer. Such networks can be very large

In some instances sequence reconstruction is on a supercomputer

The results will be presented in a graphical, interactive format.

Low confidence regions that are persistent will be indicated as such on a macro, chromosome by chromosome report of the regions sequenced. The confidence assigned to each base will be available, which is not the case in present methods.

Avoiding Mismatch Errors

Conditions will be stringent enough to prevent a 5 mer mismatch from hybridising. Furthermore, markers can be used to label mismatches or methods can be used to destroy mismatches, for example, the mismatch repair system of Escherichia coli, provides proteins, MutL, MutH and MutS which singly or in combination can be used to detect the site of a mismatch; T4 endonuclease IV can also do this. In addition treatment by tetraethyammonium chloride/potassium permanganate, followed by hydroxylamine can cleave the site of mismatch and this will be seen as a contraction in the DNA. It is likely that mismatches will only occur when a 6 mer is stabilised by flanking contiguous stacking oligonucleotides. This effect can be minimized by making oligonucleotides in which one end is phosphorylated (disrupts intimate coaxial stacking) or by adding a bulky group at the end. Depending on the algorithm mismatches may be tolerated especially where there is a well defined set of rules that describe mismatching behaviour.

The oligonucleotide probes may be detected by virtue of Fluorescence Resonance Energy Transfer (FRET) interactions with a DNA stain staining the DNA Polymer (see Howell W M, Jobs M, Brookes A J 2002 Genome Res. September;12(9):1401-7). This drastically reduces signal from non specific interactions of the probes withn the surface because only those probes which are within around 10 nm of the DNA polymer will undergo FRET.

For complete de novo sequencing, for example of organisms where no reference sequence is available, the experimental procedure is exactly the same but the task of the algorithm is greater. Supercomputers may be needed fro sequence reconstruction depending on the quality of data that is obtained.

The data is deconvoluted for ordering along the molecule and data about order and approximate distance from other probes is taken into account. A list with orders is then present to a sequencing by hybridisation algorithm. In one example of the reconstruction strategy the algorithm then splits the regions of the genome into a series of overlapping segments and computes the sequencing from the hybridisation data from each area, matching to the draft genome sequence where available, assigning probabilistic scores to the sequence data. The data is presented (e.g. via a colour chart) indicating regions of high certainty and regions of lower certainty. The regions of high certainty can be used in genetic studies.

The results are also cross-validated by Sanger sequencing technologies and with this comparison a heuristic or knowledge based system will be built up over time, enabling more accurate sequence. The aim would be to get confidences higher than error rates for common enzymes, eg. 99.9% confidence. Ultimately the sequencing may be run in parallel with other whole genome sequencing technologies to further increase confidence.

With this method it is possible that unless specific measures are taken algorithms can e be s confounded by heterozygocity over the regions. Therefore it will be preferable to use bialllelic probes to isolate haplotype tags which seed a region of linkage disequilibrium. This information about the haplotype structure of the geneome will soon become available through international efforts. Two-colour gene expression analysis

The Experimental Apparatus

The edges of the area surrounding the array are raised so that addition and removal of fluids can take place (e.g a microtitre set-up; low intrinsic fluorescene glass bottomed plates area available, e.g. from Whatman Polyfiltronics or custom made glass). Alternatively, the array substrate is sealed to a reaction cell (e.g. Teflon or Teflon coated which makes a good seal with glass) with inlet and outlet ports. Where information from single dye molecules is required, the microscopy set up will be TIRF, preferably with ulsed lasers and time gated detection, with full gamut of measures taken to minimise fluorescence background. Where the probes are labelled with fluorspheres then epi-fluorescence microscopy and excitation with a 100 W mercury lamp can be used. Where the analysis is with AFM, then nanoparticles of different sizes cna be used for labelling, analysis will be with tapping mode in Air and a liquid cell will be used for flowing in reagents and washing the array.

Experimental Procedures

Hybridise target to array (ASP method as described for lambda DNA above). Use as much target DNA as can be tolerated in the reaction mix for example, at least 10 ug of restriction digested DNA or if whole genome amplification by random primer labelling has been done then the amount of DNA obtained after amplification of as little as 500 ng of starting DNA, can be used.

Optionally instead of ligation, the captured target is chemically attached to the surface after hybridisation

Preparation and Marker Labelling of Array

The digoxygenin can be added to the array oligonucleotide during their synthesis. Once the target has hybridised a signal amplification reaction can be performed on the digoxygenin so that the point of array capture can be identified

Block slide with milk protein supernatant in PBS/Tween 20 (10″ at room temperature) and wash with PBS/Tween

1^{st} Antibody layer Add Mouse Anti-Digoxygenin Antibody (Roche) diluted 1/250 in milk protein+PBS for. Leave 30″ at RT in the dark thene do PBS/Tween washes

2^{nd} Antibody layer Add Goat Anti-Mouse Alexa Fluor 488/520 (Molecular Probes) 1/50 dilution in milk proein+PBS. Leave 30″ at 37 C in dark. Do PBS/tween wash followed by a PBS wash Dry slide (for example with gentle forced air)

The target Genomic DNA is stained with YOYO-1 (Moecular Probes) in a 1 in 1000 or 1 in 2000 dilution (other DNA labels might be used depending on wavelength of labelling of oligonucleotide probes and markers and the available filters and laser lines)A CCD image of the array is taken before the sequencing reactions begin.

Annealing of Oligonucleotide Sets and Detection

The DNA array is placed on a temperature control device such as a thermocycler fitted with a flat block

Hybridisation can be done in 3.5 M Tetramethyl ammonium Chloride that reduce the effects of base composition (see section D above for a list of other possible buffers) in which case all annealing will be done at one or two temperatures. Hybridisation of short oligonucleotides with 4-6 SSC.

Add first set of oligonucleotide probes at a concentration between 1 nM-1 uM depending oligonucleotide length and chemistry

Concentrations can be adjusted so that some but not all sample molecules give signal (for example, optimised so that 1 in 12 oligonucleotide give a signal with a particular oligonucleotidene sequence).

This is done at a temperature that is optimal for the Tm. For DNA oligonucleotides this may be between 0 and 10 degrees C. For LNA/PNA oligonucleotides a higher temperature can be used e.g. room temperature. If for example an enzymatic reaction is performed e.g. ligation to random 9 mers then a higher reaction temperature e.g 65 degrees C. with Tth DNA ligase, can be used.

Rolling circle amplificaton can be used to amplify signal from each probe. In this example the probes are bipartite, with sequecnce complementary to target and circuler oligonucleotide round which polymerisation extends using Sequenase enzyme and single stranded bindig protein (SSB) essentially as described (Zhong et al PNAS 98: 3940-3945).

Also bipartites probes may comprise one portion which is complementary to the target and a second portion which is a partner to a molecule attached to a fluorecent label. The partners may be antibody-antigen interactions or they may be complementary olgounucleotide interactions.

Denaturing Oligonucleotides

Some sequencing strategies require oligonculetodes or at least ther labels to be removed. Oligonucleotides can be denatured under gentle agitation by one or more of the following treatments

*High Stringency buffer e.g. 0.1×SSC or

High Stringency buffer e.g. 0.1×SSC followed by water or Tris EDTA or Alkali buffer, 100 mM Sodium Carbonate/Hydrogen carbonate, room temperature

*And/or Heat to 37

And/or Heat to 37 to 70 degrees C

Harshness of treatment that can be tolerated is determined by the number of cycles that need to be performed.

It is not essential to remove all probes. But it is important to image which probes remain binding after treatment

Less harsh treatments labelled with asterisk above are preferred.

The addition of glycerol can aid in keeping the DNA in good condition

Removal of oligonucleotides by enzymatic treatment would also be preferable as this is less harsh.

The sequence can be computed from the hybridisation data from each area, matching to the draft genome sequence where available assigning probabilistic scores. The data is presented with a colour chart indicating regions of high certainty and regions of lower certainty. The regions of high certainty can be used in genetic studies.

Sequencing may be by any of the sequencing approaches described in this document. Alternatively the arrays of this invention generate substrates highly suitable for sequencing by synthesis.

Gene (mRNA) Expression Analysis

Single molecule arrays of two types can be prepared for gene expression analysis. The first is oligonucleotide arrays, which are either synthesised in situ or are pre-synthesised and spotted. The second is by spotting of cDNAs or PCR product. The former can be spotted essentially as described. For the latter the optimal concentration to spot the oligonucleotides to get single molecule detection with a method of choice needs to be determined empirically, as already described. Following this cDNA arrays are spotted essentially as described onto for example, aminosilane slides using 50% DMSO as spotting buffer.

Preparing Fluoresenctly Labeled cDNA (Probe) by Brown/DeLisi Protocol or an Adaptation Thereof:

For single molecule counting based on analysis of a single dye molecule, the CDNA must be primer labelled where the primer carries a single dye molecule or alternatively carries a single biotin molecucle or is aminated for attachment to single beads.nanoparticles.

In a modification, the cDNAs are labelled with incorporation of ddNTPs so that short fragments are created.

To anneal primer, mix 2 ug of mRNA or 50-100 μg total RNA with 4 ug of a regular or anchored oligonucleotide-dT primer in a total volume of 15.4 ul:

| | Cy3 | Cy5 | |
|---|---|---|---|
| mRNA (1 γ/λ) | x λ | Y λ | (2 μg of each if mRNA, |
| | | | 50-100 μg if total RNA) |
| Oligonucleotide- | 1 λ | 1 λ | (Anchored: 5′- |
| dT (4 γ/λ) | | | TTT TTT TTT TTT |
| | | | TTT TTT TTV N-3′) This |
| | | | primer may be labelled at |
| | | | the 5′ end with a dye |
| | | | moleucle e.g Cy3 or Cy5. |
| | | | This can be specified when |
| | | | the oligonucleotide is |
| | | | ordered from e.g.Oswel, |
| | | | Southampton, UK) |
| ddH₂O (DEPC) | to 15.4 λ | to 15.4 λ | |
| Total volume: | 15.4 λ | 15.4 λ | |

Heat to 65° C. for 10 min and cool on ice.

Add 14.6 μL of reaction mixture each to Cy3 and Cy5 reactions:

| | | . . . Unlabeled | | | Final |
|---|---|---|---|---|---|
| Reaction mixture | λ | . . . dNTPs | Vol. | | conc. |
| 5× first-strand buffer* | 6.0 | dATP (100 mM) | 25 | uL | 25 mM |
| 0.1 M DTT | 3.0 | DCTP (100 mM) | 25 | uL | 25 mM |
| Unlabeled dNTPs | 0.6 | DGTP (100 mM) | 25 | uL | 25 mM |
| Cy3 or Cy5 (1 mM, | 3.0 | DTTP (100 mM) | 10 | uL | 10 mM |
| Amersham)** | | | | | |
| Superscript II (200 U/uL, | 2.0 | ddH2O | 15 | uL | |
| Gibco BRL) | | | | | |
| Total volume: | 14.6 | Total volume: | 100 | uL | |
| | λ | | | | |

*5× first-strand buffer: 250 mM Tris-HCL (pH 8.3), 375 mM KCl, 15 mM MgCl2)
**Fluorescent nucleotides are omitted when a labelled primer is included or when labelling is through a labelled ligation primer (as described below)
Incubate at 42° C. for 1 hr.

Add 1 λ SSII (RT booster) to each sample. Incubate for an additional 0.5-1 hrs.

Degrade RNA and stop reaction by addition 15 μl of 0.1 N NaOH, 2 mM EDTA and incubate at 65-70° C. for 10 min. If starting with total RNA, degrade for 30 min instead of 10 min.

Neutralize by addition of 15 μl of 0.1 N HCl.

Add 3801 μl of TE (10 mM Tris, 1 mM EDTA) to a Microcon YM-30 column (Millipore). Next add the 60 μl of CyS probe and the 60 μl of Cy3 probe to the same microcon. (Note: If re-purification of cy dye flow-through is desired, do not combine probes until Wash 2.)

WASH 1: Spin column for 7-8 min. at 14,000×g.

WASH 2: Remove flow-through and add 450 ul TE and spin for 7-8 min. at 14,000×g. It is a good idea to save the flow trough for each set of reactions in a separate microcentrifuge tube in case Microcon membrane ruptures.

WASH 3: Remove flow-through and add 450 ul 1× TE, 20 μg of Cot1 human DNA (20 μg/μl, Gibco-BRL), 20 μg polyA RNA (10 μg/μl, Sigma, #P9403) and 20 μg tRNA (10 μg/μl, Gibco-BRL, #15401-011). Spin 7-10 min. at 14,000×g. Look for concentration of the probe in the microcon. The probe usually has a purple color at this point. Concentrate to a volume of less than or equal to the 28 ul . These low volumes are attained after the centre of the membrane is dry and the probe forms a ring of liquid at the edges of the membrane. Make sure not to dry the membrane completely!

Invert the microcon into a clean tube and spin briefly at 14,000 RPM to recover the probe. Using a 22×60 mm coverslip use a total volume of 35 ul composed of 28 ul Probe and TE, 5.95 ul 20×SSC, 1.05 ul 10% SDS

*20×SSC: 3.0 M NaCl, 300 mM NaCitrate (pH 7.0)

Adjust the probe volume to 28 ul column above.

For final probe preparation add 4.25λ 20×SSC and 0.75λ 10% SDS. When adding the SDS, be sure to wipe the pipette tip with clean, gloved fingers to rid of excess SDS. Avoid introducing bubbles and never vortex after adding SDS.

Denature probe by heating for 2 min at 100° C., and spin at 14,000 RPM for 15-20 min. Place the entire probe volume on the array under a the appropriately sized glass cover slip. Hybridize at 65° C. for 14 to 18 hours in a custom slide chamber with humidity maintained by a small reservoir of 3×SSC (spot around 3-6 λ 3×SSC at each corner of the slide, as far away from the array as possible).

II. Washing and Scanning Arrays:

Ready washes in 250 ml chambers to 200 ml volume as indicated in the table below. Avoid adding excess SDS. The Wash 1A chamber and the Wash 2 chambers should each have a slide rack ready. All washes are done at room temperature.

| Wash | Description | Vol (ml) | SSC | SDS (10%) |
|---|---|---|---|---|
| 1A | 2× SSC, 0.03% SDS | 200 | 200 ml 2× | 0.6 ml |
| 1B | 2× SSC | 200 | 200 ml 2× | — |
| 2 | 1× SSC | 200 | 200 ml 1× | — |
| 3 | 0.2× SSC | 200 | 200 ml 0.2× | — |

Blot dry chamber exterior with towels and aspirate any remaining liquid from the water bath. Unscrew chamber; aspirate the holes to remove last traces of water bath liquid.

Place arrays, singly, in rack, inside Wash I chamber (maximum 4 arrays at a time). Allow cover slip to fall, or carefully use forceps to aid cover slip removal if it remains stuck to the array. DO NOT AGITATE until cover slip is safely removed. Then agitate for 2 min.

Remove array by forceps, rinse in a Wash II chamber without a rack, and transfer to the Wash II chamber with the rack. This step minimizes transfer of SDS from Wash I to Wash II. Wash arrays by submersion and agitation for 2 min in Wash II chamber, then for 2 min in Wash III (transfer the entire slide rack this time).

Spin dry by centrifugation in a slide rack in a Beckman GS-6 tabletop centrifuge at 600 RPM for 2 min

Analyse arrays immediately on a single molecule sensitive detector such as the Light station (Atto-tec).

Instead of performing step 1 in the above protocol with labelled target cDNA, because the requirement of the assay of this invention is a single dye molecule, a target labelling procedure can be ommitted. Thence, unlabelled cDNA or Poly A mRNA or total RNA can be hybridised directly. This is then followed by hybridisation of either:

A random library of n-mers (e.g 8-10 m mers) which are labelled 5′ phosphorylated and 3′ labelled are ligated to arrayed sequence specific oligonucleotidenculeotide probes (e.g to as can be made by Febit or Xeotron, or can be spotted), templated by the target mRNA

A library of sequence specific probes which are labelled as above are ligated to oligonucleotides in an n-mer array, templated by the target mRNA

Where Total RNA is used blocking sequences are used to mop up ribosomal RNAs, small nuclear RNAs and transfer RNAs.

In the above process, several dye molecules are incorporated into each single cDNA molecule. If the density of the array is low enough signals from a single species can be distinguished by their spatial co-localization and that they are a single colour. The single molecules will form a Poisson distributon so there will be some molecules that cannot be resolved but these will be minimal if the spacing is far enough apart. In an alternative method the oligonucleotided(I) primer s end labelled. This can be labelled ith a single dye molecule, multilabelled with dendrimers or labelled with a Fluospheres (Molecular Probes).

The results of the assay are based on the ratio of the number of molecules (or colocalized sets of molecules) counted for each of the populations.

Single Molecules can be counted on low density arrays when using small number of cells (˜1000) and when using normal amounts (e.g 10⁶). Alternatively arrays, can be single molecule arrays by functionalisation. In this case, small amounts of sample material 100-1000 cells must be used to achieve the single molecule functional array which can be used to count single molecules.

Determining the levels of translated proteins by analyzing mRNA linked to polysomes as Brown et al.

RNA is extracted by methods known in the art

Ligand-Protein Binding Assay on Single Molecule Chemical Arrays

Aminosilane (APTES) slides from Asper biotech (Estonia) are derivatised (according to Gavin MacBeath, Angela N. Koehler, and Stuart L. Schreiber J. Am. Chem. Soc., 121 (34), 7967-7968, 1999) to give surfaces that are densely functionalized with maleimide groups. To achieve this, one face of each slide is treated with 20 mM N-succinimidyl 3-maleimido propionate (Aldrich Chemical Co., Milwaukee, Wis.) in 50 mM sodium bicarbonate buffer, pH 8.5, for three hours. (This solution is prepared by dissolving the N-succinimidyl 3-maleimido propionate in DMF and then diluting 10-fold with buffer). After incubation, the slides are washed several times with milliQ water, dried by centrifugation, and stored at room temperature under vacuum until further use. A dilution series of biotin molecule is arrayed. Upon binding of cy3-labelled streptavidin or a 20 nm Streptavidin coated Fluosphere to the array, the optimal dilution for detecting single molecules is established. A single molecule binding assay can then be conducted. Where Streptavidin is labeled with a single cy3 dye, the single step photobleaching characteristics of the dye are sufficient to indicate single molecules.

Protein-Ligand Binding Assay on Single Molecule Protein Arrays

Avidin , Streptavidin, Neuravidin are arrayed on a surface, for example onto a biotin-derivatized surface. Fluorescent semiconductor nanocrystals coated with biotin molecules (Quantum Dot Corp) are then interacted with the Proteins using the Quantum Dot buffer supplied by the vendor at a temperature between room temperature and 45 degrees. A 1 hour reaction at 45 degrees is sufficient. Arrayed single molecules are then interrogated. In an alternative example, the Avidin and derivatives are also previously labelled e.g with different dyes or Fluospheres (Molecular Probes Copr, Oreg.) according to which they can be distinguished. The assay can then be carried out on arrays spreads of the avidin and derivatives.

Protein:Protein/Antigen:Antibody Binding on Protein Arrays

The following is adapted from the procedure of Haab and Brown:

Preparation of Protein Analyte Solutions

Protein solutions and NHS-ester activated Cy3 and Cy5 solutions (Amersham) are prepared in a 0.1 M pH 8.0 sodium carbonate buffer. The protein and dye solutions are mixed together so that the final protein concentration is 0.2-2 mg/ml and the final dye concentration was 100-300 μM. Normally approximately 15 g protein is labeled per array. The reactions are allowed to sit in the dark for 45 min and then quenched by the addition of a tenth volume 1 M pH 8 Tris base (a 500-fold molar excess of quencher). The reaction solutions are brought to 0.5 ml with PBS and then loaded into microconcentrator spin columns (Amicon Microcon 10) with a 10,000 Da molecular weight cut off. After centrifugation to reduce the volume to approximately 10 μl (approximately 20 min), a 3% non-fat milk blocking solution is added to each Cy5-labeled solution such that 25 μl milk is added for each array to be generated from the mix. (The milk had been first spun down as above.) The volume is again brought to 0.5 ml with PBS and the sample again centrifuged to ˜10 μl. The Cy3-labeled reference mix is divided equally among the Cy5-labeled mixes, and PBS is added to each to achieve 25 μl for each array. Finally, the mixes are filtered with a 0.45 μm spin filter (Millipore) by centrifugation at 10,000×g for 2 min.

Binding to Array

Without allowing the array to dry, 25 μl dye-labeled protein solution is applied to the array surface and a 24×30 mm cover slip is placed over the solution. The arrays are sealed in a chamber with an under-layer of PBS to provide humidification, after which they are left at 4° C. for 2 h. The arrays are dipped briefly in PBS to remove the protein solution and cover slip, and are then allowed to rock gently in PBS/0.1% Tween-20 solution for 20 min. The arrays are then washed twice in PBS for 5-10 min each and twice in H₂O for 5-10 min each. All washes are at room temperature. After spinning to dryness in a centrifuge equipped with plate carriers (Beckman) or by removing moisture by forced air, the single molecule protein arrays are ready for analysis.

Measuring Physico-Chemical Properties and Interactions

Scanning probe microscopes can be used to measure physicochemical properties of molecules. An AFM tip may be made hydrophobic and its interaction with arryed proteins can be measured. In addition a chemical (Chemical Force Microscopy) pr biomolecule can be attached to the tip of an AFM and its interactions with an arrayed protein or DNA molecule can be analysed. A 2-dimensional array of force curves can be obtained by using an AFM developed by Asylum research. Different aspects of the interactions, such as electrostics can be determined from these force curves by those trained in the art. The properties of a given protein can be learnt and stored in a look up table. During or after force mapping, comparisons are made with the look up table to see if the ascertained features match those in the look up table. Depending on the match the identity of a protein molecule can determined. Radmacher et al Science 1994 265: 1577 describe the use of AFM force measurements for analysing the properties of an enzyme molecule. The sample protein molecules can be arrayed in a manner that those molecules with certain features lie at certain regions of the array. For example, proteins may be immobilised on a surface bearing a pH gradient on which different proteins bind to different pH locations according to their corresponding Isoelectic point (Wasch-Mesthgeet al Scanning 2000 22:380).

Another method for fingerprinting single protein molecules is by taking advantage of the massive enhancement in Raman signal due to surface enhancement by metal clusters. Colloidal gold (Sigma) is added to a gold coated microscope slide (Erie Scientific) and clusters are allowed to form to generate a SERS (Surface Enhanced Raman. Spectroscopy) active surface. Raman spectrum is obtained in the near Infra-red wavelength range, using a CCD camera and a spectrograph. The concentration of target molecule required so that only a single target protein molecule is immobilised per cluster is determined by testing a range of sample concentrations. The spectra for each protein of interest are obtained and stored in a look up table. Then a mixture of proteins is arrayed onto a surface containing metallic clusters, at a dilution that a single molecule will bind to a single cluster. Raman spectrum are then obtained from different locations on the surface (using a X_Y translation of the sample for example). Each spectrum is compared to fingerprints in the look up table, if a match is found then presence of that particular protein in the sample is indicated The look up tables are stored in computer memory and comparisons with the look up table may utilise neural network and fuzzy logic software as known in the art.

Microscopy and Imaging

Fluorescence Detection Schemes and Instrumentation

The images of the molecules are projected onto the array of a Charge-couple device (CCD) camera, from which they are digitized and stored in memory. The images stored in memory are then subjected to image analysis algorithms. These algorithms can distinguish signal from background, monitor changes in signal characteristics, and perform other signal processing functions. The memory and signal processing may be performed off-line in a computer, or in specialized digital signal processing (DSP) circuits controlled by a microprocessor.

When individual molecules within the microarray spot are analysed directly, then wide field CCD imaging is used. CCD imaging enables a population of single molecules distributed 2-dimensionally on a surface to be viewed simultaneously. Although microarray imagers based on epifluorescence illumination and wide field imaging are available, the optics and range of stage movement of these instruments does not enable single molecules to be monitored across large areas of the slide surface. Typically, wide-field illumination schemes may involve illumination with a lamp, a defocused laser beam or by an evanescent field generated by Total Internal Reflection of a laser beam. The field that can be viewed is determined by the magnification of the objective, any magnification due to the C-mount and, the size and number of pixels of the CCD chip. Typically, a microarray spot can be viewed by either a 40× or 60× objective depending on CCD camera and C-mount. Therefore to view large regions of a slide (several cm²) multiple images must be taken. A low noise high sensitivity camera is used to capture images. There are several camera models that can be used; Cooled Micromax camera (Roper scientific) controlled by MetaMorph (also MetaView software; both from Universal Imaging). MetaMorph can be run on a Dell OptiPlex GX260 personal Computer.

The following CCD set ups can be used I-PentaMAX Gen m; Roper Scientific, Trenton, N.J. USA) or cooled (e.g. Model ST-71 (Santa Barbara Instruments Group, Calif., USA); ISIT camera composed of a SIT camer a(Hamamatsu), an image intensifier and (VS-1845, Video Scope Intematinal, USA) and stored on S-VHS videotape. Video taped images are processed with a digital image processor (Argus-30, Hamamatsu photonics). Gain setting are adjusted depending on camera and brightness of signal.

The movement form one field of view to another can be done by attaching the substrate on a X-Y translation stage (Prior Scientific).

Feature Recognition and Single Molecule Imaging

MetaMorph's optional microarray module and a low magnification objective are used to locate spots before taking a CCD image of each of the spots using higher magnification.

As the signal from the spots containing singly resolvable molecules is very low under low magnification, a marker dye, which emits at a different wavelength to the sample emission should be included in the spots to help locate them. The objectives need to be of high numerical aperture (NA) in order to obtain good resolution and contrast. The integration of an autofocusing capability within the procedure to maintain focus as the slide is scanned, is useful especially when Total Internal Reflection Fluorescence microscopy (TIRF) is employed. Software can be used to control Z movement (integral to motorized microscopes) for the purpose of autofocusing (e.g. MetaMorph). Images of microarray spots can be obtained by x-y movements of the sample stage (e.g. using Prior Scientific's Proscan stage under MetaMorph control). To avoid photobleaching it is advisable to use a shutter (e.g. from Prior Scientific) to shut of illumination while moving from one spot to another. A controller can be used to control X-Y stage, the filter wheels and shutter, (eg Prior Scientific ProScan).

Once the spots are found, their coordinates are recorded by the software controlling the instrument and then after each base addition, a CCD image is taken of each spot of the microarray.

In addition to the instrument being used for looking at a microarray where template molecules have been captured by probes, a large number of samples can be gridded (as a microarray) to form an array of arrays and then the instrument can be used to analyse each array. The samples may be individual nucleotide populations or a set of differentially labeled nucleotide populations.

Two imaging set ups, Total Internal Reflection Fluorescence microscopy (TIRF) and epi-fluorescence microscopy have been used.

Epi-Fluorescence Imaging

Images of single molecules labeled with a single dye molecule can be obtained using a standard epi-fluorescence microscopy set up, using high NA objectives and a high grade CCD camera. However, the image can be hazy. In order to obtain a clearer image it is preferable to use deconvolution software to remove the haze. Deconvolution modules are available as drop-ins for MetaMorph software. When the single molecules are labeled with nanoparticles the camera and objectives may be of a lower grade.

Total Internal Reflection Microscopy (TIRF)

TIRF enables very clean images to be obtained by creating an evanescent field which decays exponentially from the surface, for example using off the shelf system for Objective style TIRF (such as those produced by Olympus or Nikon). A full description can be found in the brochure at the following website: www.nikon-instruments.com/uk/pdf/brochure-tirf.pdf

Objective style TIRF can be used when the sample is on a coverslip. However, it is not compatible when the sample is on a microscope slide. For this Prism type TIRF must be used (See Light Microscopy in Biology, A practical Approach Ed. AJ Lacey OUP). In addition a high NA condensor can be used to create TIRF on a microscope slide.

There are two configurations that can be used with TIRF. The first is the Prism method and the second is the objective method.

The objective method is supported by Olympus Microscopes and application notes are found at the following web site: http://www.olympusmicro.com/primer/techniques/fluorescence/tirf/olympusaptirf.html

The Prism method below is described in Osborne et al J. Phys. Chem. B, 105 (15), 3120-3126,2001.

This instrument consists of an inverted optical microscope (Nikon TE200, Japan), two color laser excitation sources, and an Intensified Charge Coupled Device (ICCD) camera (Pentamax, Princeton Instruments, NJ). A mode-locked frequency-doubled Nd:YAG laser (76 MHz Antares 76-s, Coherent) is split into two beams to provide up to 100 mW of 532-mn laser light and pump a dye laser (700 series, Coherent) with output powers in excess of 200 mW at 630 mn (DCM, Lambda Physik). The sample chamber is inverted over a ×100 oil immersion objective lens and a 60 fused silica dispersion prism optically coupled to the back of the slide through a thin film of glycerol. Laser light is focused with a 20-cm focal length lens at the prism such that at the glass/sample interface it subtends an angle of approximately 68 to the normal of the slide and undergoes total intemalreflection (TIR). The critical angle for a glass/water interface is 66. The footprint of the TIR has a 1/e2 diameter of about 300 m. Fluorescence produced by excitation of the sample with the surface-specific evanescent wave is collected by the objective, passed through a dichroic beam splitter (560 DRLP, Omega Optics), and filtered before imaging onto the ICCD camera. Images are recorded by using synchronized 532 nm excitation withdetection at 580 mn (580DF30, Omega) for TAMRA labeled substrates and 630 nm excitation with detection at 670 nm (670DF40, Omega) for Cy5 labeled probes. Exposure times are set between 250 and 500 ms with the ICCD gain at maximum (1 kV). The laser powers at the prism are adjusted to 40 mW at both laserwavelengths.

Although the above describes use of the system on an inverted microscope, an upright microscope can also be configured in an appropriate way, for example Braslavsky I, Hebert B, Kartalov E, Quake SR. Proc Natl Acad Sci U S A.100:39604. (2003)

Multi-Colour Single Molecule Imaging

When the single molecule technique involves different fluorescen labels added sequentially, then a single CCD image can be taken for each. However, if each nucleotide is differentially labeled (i.e. each nucleotide type is labelled with a different fluorophore) and added simultaneously, then the signal from each of the differerent fluorophores needs to be acquired distinguishably. This can be done by taking four separate images by switching excitation/emission filters. Alternatively, an image (Wavelength) splitter such as the Dual View (Optical Insights, Santa Fe, N.Mex.) or W View (Hamamatsu, Japan) which direct the light through two separate bandpass filters with little loss of light between them, can be used for imaging two different wavelenghts onto different portions of a CCD chip. Alternatively the light can be split into four wavelengths and sent to the four quadrants of a CCD chip (e.g Quad view from Optical Insights). This obviates the need to switch filters using a filter wheel. A MetaMorph drop-in for single image dual emission optical splitters can also be employed.

Analysing Single Molecules Randomly Distributed on a Surface

As an alternative to microarray spot finding prior to single molcule imaging and for implementations where the single molecules to be analysed are not organised within the spatially addressable microarray spots, a series of images of the surface can be taken by x-y translation of the slide. A super-wide field image is then composed by stitching each of the images together.

SNOM (Scanning Near-Field Optical Microscopy

SNOM (e.g BioLyser SNOM (Triple-O Potsdam, Germnay)) can be used for near field optical imaging, allowing molecules at closer spacings to be individually resolved.

Stains and Antifade

The following oxygen scavenging solution can be used to minimise photobleaching when single molecule analysis is done in solution: Catalase (0.2 mg/ml), Glucose oxidase (0.1 mg/ml), DTT (20 mM), BSA (0.5 mg/ml), Glucose 3 mg/ml. This can be added to the buffer solution that is being used in the experiment.

Adding 20-30% beta-mercaptoethanol to a solution will attenuate photobleaching. DNA can be stained by using a variety of dyes available form Moelcular Probes (Oregon) e.g. YOYO-1, POPO-3 and SYBR Gold, used at manufacturers recommended concentrations.

AFM

Images cn be obtained by using a Multimode IIIa with a nanoscope IV controller and Si cantilever tips (Veeco, Santa Barbara, Calif.). This is placed on an active isolation system (MOD1-M, Halcyonics, Gottingen, Germany). Typical imaging parameters are 60-90 Hz resonant frequency, 0.5-1V oscillation amplitude, 0.3-0.7V setpoint voltage, 1.5-2 Hz scan rate.

Image Processing, Single Molecule Counting and Error Management

The above can be done using algorithms of any of the type in the detailed description of the invention. In addition below is an example of how to do single molecule counting using simple commercial software.

The objective is to use image analysis to count and determine the confidence in putative signals from single molecules within a microarray spot. The image processing package SigmaScanPro is used to automate single molecule counting and measurement. The procedure described here, or modifications of it, can be used for simple single molecule signal counting or more complex analyses of single molecule information, multi-colour analysis and error mangement.

The microarray spot or array region of interest image is captured using a CCD camera, such as the I-PentaMAX Genm or Gen IV(Roper Scientific) and an off-the-shelf frame grabber board. The single molecules are excited by laser in a TIRF configuration. Using a 100× objective and spots of approximately 200 microns in diameter.

The image is spatially calibrated using the Image, Calibrate, Distance and Area menu option. A 2-Point Rescaling calibration is performed using micron units. Single molecule areas will then be reported in square microns.

Increasing the contrast between single molecules and the surrounding region will help identify the single molecules by thresholding. Image contrast is improved by performing a Histogram Stretch from the Image, Intensity menu. This procedure measures the grey levels in the image. The user then “stretches” the range of grey levels with significant magnitude over the entire 255 level intensity range. In this case moving the Old Start line with the mouse to an intensity of 64 will eliminate the effect of the insignificant dark gray levels and improve the contrast.

The single molecules can be identified by thresholding the intensity level to fill in the darkest objects. This is done by selecting Threshold, Intensity Threshold from the Image menu.

Under certain spotting conditions (e.g. 1.5 M Betaine 3×SSC onto enhanced Aminoslinae slides as well as in 50% DMSO buffer under certain conditions) the spot has a thin but discernably bright ring round the edge. This can be used to define the area to be processed. This ring can be removed from contributing to the data by using image overlay layer math to intersect the single molecule signals with an overlay plane consisting of the interior of the ring. The overlay is created by filling light pixels in the interior of the spot and selecting out the ring by thresholding. Set the Level to be 180 and the option to select objects that are lighter than this level. Select the Fill Measurement mode (paint bucket icon) and left click in the interior of the plate to fill it. Set the source overlay to red in the Measurements, Settings, Overlays dialog. There are “holes” in the red overlay plane that are not filled since they contain bright pixels from the single molecules. To fill them select Image, Overlay Filters and select the Fill Holes option. Let both the source and destination overlays be red. The red circular overlay plane contains the green bacterial colonies.

The overlay math feature is used to identify the intersection of the red and green overlay planes. From the Image menu select Overlay Math and specify red and green to be the source layers and blue to be the destination layer. Then AND the two layers to obtain the intersection.

The blue pixels overlay the single molecule that can now be counted. Select the blue overlay plane as the source overlay from the Overlays tab in the Measurement Settings dialog. Select Perimeter, Area, Shape Factor, Compactness and Number of Pixels from the Measurements tab in the Measurements Settings dialog. Then measure the single molecule signals by using Measure Objects from the Measurements menu. The single molecule signals can be arbitrarily numbered and the corresponding measured quantities placed into an Excel Microsoft) spreadsheet

A macro is written to perform this for each spot in the array.

The microarray slide is translated relative to the CCD by a X-Y translation (Prior Scientific) stage with images taken approximately every 100 micron spacings.

The example given here is for end-point analysis. However, for enhanced error discrimination real time analysis may be desirable, in this case a wider field images can be taken of the whole array by the CCD camera under lower magnification and enhanced by image processing. However, in most cases, a time window after the start of the reaction will have been determined within which the image should be acquired to gate out errors, which may occur early (non specific absorption) and late (mismatch interactions) in the process.

Adobe Photoshop software contains a number of image processing facilities which can be used and more advanced plug-ins are available. The Image Processing Toolkit is available which Plug-in to Photoshops, MicroGrafx Picture Publisher, NIH Image and other programs is available from Quantitative Image Analysis.

Biosensors

Biosensor in which single molecules are detected by fluorescence

The molecular array, an excitation source and a detector CCD are integrated into a small device. The Molecualr array is synthesised on a substrate in which an evanescent field is created by a waveguide.

Biosensor in Which Single Molecules are Detected by Conductivity

An integrated biosensor is created in which the molecules of the array are attached to electrodes. A voltage source for the electrodes is provided and electronic circuitry for detection.

Additional Elements of the Integrated Sensor

In addition, optionally means for any or all of the following can be included in the sensor microprocessor, memory, hardware-based signal processing (circuitry for processing the electrical change generated by at least one sensing element into a resulting output signal indicative of feature analysed), software-based signal processing; software-based processing of results, display of results; transmitting antennae and optionally receiver for communication with a local or remote computer carrying central database on a remote computer, computer memory. The microprocessor includes suitable memory as well as processing. The device can further include a set of internal batteries for powering the processor and the sensing array.

The microprocessor may electronics include an analog-digital (A/D) converter as well as resident control and timing circuitry which is used in conjunction with a reference crystal in order to detect the amount of electrical change by each of the sensing elements of the array for processing, such as comparing to a stored look-up table and then outputting the results to an LCD or other suitable display.

Coating of Palladium

A saturated Pd solution is prepared by dissolving Palladium in aqueous buffer or other solvent. This is then placed on the DNA sample and allowed to react. Then reducing solution containing dimethylamine borane is added. Excess reagent is washed away or diluted out.

Coating of Silver

Being a polyanion the DNA bridge is loaded with silver ions by NA+/AG+ ion exchange using 0.1 MagNO3basic aqueous solution (ammonimum hydroxide pH 10.5) The silver ion is reduced using basic hydroquinone solution (0.05 M ammhyydroxideph10.5) to form small silver aggregates bound to DNA . The DNA wire is developed using an acidic solution ph 3.5 citrate buffer of hydroquinone 0.05 M and silver ions 0.1 M under low light conditions

Aldehyde Mediated Metalization

Kerene et al (Science 297: 72 , 2002) have described a procedure in which first reducing agent is coupled to the DNA polymer by incubating the DNA with glutaraldehyde. AgNO3 in ammonia buffer is then added and the reduction of silver by the DNA bound aldehyde leads to the formation of microscopic Ag aggregates along the DNA polymer. The silver aggregates serve as catalysts for subsequent gold deposition to produce continuous gold wires.

The same procedure can be used for metallization of a microarray spot (see below).

Metallizing DNA with Zinc to Form M-DNA

A. Rakitin et al (Physical Review Letters 86:3670-3673) have described an approach which involves substituting the imino proton of each base pair with a metal ion, Zn²⁺ to obtain M-DNA with altered electronic properties. M-DNA is prepared in 20 mM NaBO3 buffer, pH=9.0 (or 20 mM Tris, pH=7.5) with 10 mM NaCl at 20 C and 0.1 mM Zn²⁺. This treatment can be performed before or after binding of the DNA to the array.

Gold can also be deposited using an evaporation procedure as described (Quake and Scherer Science 290:1536-1540).

Metalizing Microarray Spots

A DNA array may first be made and then metallized thereby becoming microelectrodes. This can be done by for example one of the following approaches

Glutaraldehyde treatment followed by metallization as described above

Providing a thiol or sulfhydryl group on the array probes such that colloidal gold particles interact with them. The gold particles then seeed silver enhancement. This can be done by an adaption of the strategy described by Taton et al Science 289: 1757 (2000): The Gold particles(e.g. Sigma) bind to the array probes then silver enhancer (e.g. Sigma) is added. In this process silver ions are reduced by hydroquinone to silver metal at the surfaces of the gold nanoparticles.

Adding gold particles with a positively charges surface coating such as lysine which bind by electrostatic attraction to the negatively charged nucleic acid probes This is done by adjusting th epH of the colloidal gold particles to pH 7 and then adding lysine molecules. In a test experiment, continuity between two separated microarray spots due to a metalized DNA bridge can be checked using mobile electrodes and an electrometer.

Fabrication of an Array of Microelectrodes and Deposition of Probes

An array of microelectrodes were fabricated by electron beam evaporation of chromium and gold onto silicon wafers or glass surfaces previously patterned with an organic photoresist using conventional UV light photolithography. After the photoresist is removed the metal is annealed by heating and cleaned by reactive ion etching. The resulting microelectrodes are connected to separate printed circuit board tracks via gold wire bonds (which may be fanned out). Electrodes in the 100 nm range can also be made by essentially the same type of procedures but using electromagnetic waves of lower wavelength). The probes may be deposited or synthesised atop of this array of microelectrodes.

The contact between the electrode and the metalized sample DNA may be improved by engineering the interface by mixing for example conjugated polymers such as polypyrrole with the nucleic acid probes on the surface.

Single molecules can be viewed on stripped fused silica optical fibres, essentially as described by Watterson et al (Sensors and Actuators B 74: 27-36 (2001). Molecular Beacons can be seen in the same way (Liu et al Analytical Biochemistry 283: 56-63 (2000)). A biosensor device can be made in which on single molecule analysis of Molecular Beacons in an evanescent field can be done.

The various features and embodiments, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology, single molecule detection or combinatorial chemistry or related fields are intended to be within the scope of the following claims.

1. A method for producing a molecular array which method comprises immobilising to a solid phase a plurality of molecules at a density which allows individual immobilised molecules to be individually resolved, wherein each molecule in the array is spatially addressable and the identity of each molecule is known or determined prior to immobilisation.

2. A method according to claim 1 wherein the molecules are applied to the solid phase by a method selected from printing, electronic addressing, or in situ synthesis by light-directed synthesis, ink jet synthesis or physical masking.

3. A method according to claim 2 wherein the molecules are applied to the solid phase by printing of dilute solutions.

4. A method for producing a molecular array which method comprises:
(i) providing a molecular array comprising a plurality of molecules immobilised to a solid phase at a density such that individual immobilised molecules are not capable of being individually resolved; and
(ii) reducing the density of functional immobilised molecules in the array such that remaining individual functional immobilised molecules are capable of being individually resolved;
wherein each individual functional molecule in the resulting array is spatially addressable and the identity of each molecule is known or determined prior to the density reduction step.

5. A method according to claim 4 wherein the density of functional molecules is reduced by cleaving all or part of the molecules from the solid phase.

6. A method according to claim 4 wherein the density of functional molecules is reduced by functionally inactivating the molecules in situ.

7. A method according to claim 4 wherein the density of functional molecules is reduced by labelling some of the plurality of molecules such that individual immobilised labelled molecules are capable of being individually resolved.

8. A method according to claim 1 wherein the immobilised molecules are present within discrete spatially addressable elements.

9. A method according to claim 8 wherein the structure of probes present in each discrete spatially addressable elements is precisely known and unintended structures are substantially absent.

10. A method according to claim 8 wherein a plurality of molecular species are present within one or more elements and each molecular species in an element can be distinguished from other molecular species in the element by means of a label.

11. A method according to claim 1 wherein the plurality of molecules which are capable of being individually resolved are capable of being resolved by optical means.

12. A method according to claim 1 wherein the plurality of molecules which are capable of being individually resolved are capable of being resolved by scanning probe microscopy.

13. A method according to claim 1 wherein the molecules are attached to the solid phase at a single defined point.

14. A method according to claim 1 wherein the molecules are attached to the solid phase at two or more points.

15. A method according to claim 1, wherein the molecules comprise a detectable label.

16. A method according to claim 15 wherein the label can be read by optical methods.

17. A method according to claim 15 wherein the label is a single fluorescent molecule or nano-particle/rod, or a plurality of fluorescent molecules or nano-particles/rods.

18. A method according to claim 15 wherein the label is a non-fluorescent molecule, nanoparticle or nanorod.

19. A method according to claim 1 wherein the molecules are selected from defined chemical entities, oligonucleotides, polynucleotides, peptides, polypeptides, conjugated polymers, small organic molecules or analogues, mimetics or conjugates thereof.

20. A method according to claim 19 wherein the molecules are cDNAs and/or genomic DNA.

21. A method according to claim 19 wherein the molecules are oligonucleotides or polynucleotides and the molecules are provided as groups of molecules, each group of molecules selectively hybridising to a different site within a target nucleic acid molecule and immobilised to the solid phase such that each group is spatially distinct from the other groups.

22. A method according to claim 21 wherein within each group, different molecular species are immobilised in discrete spatially addressable elements.

23. A method according to claim 22 wherein the different molecular species selectively hybridise to different alleles.

24. A method according to claim 21 wherein the different groups of molecules are immobilised to the solid phase such that the order of arrangement of each group relative to the other groups on the solid phase corresponds to the order of the corresponding sites in the target nucleic acid molecule.

25. A method according to claim 21 wherein the different groups are arranged along a first horizontal axis of the solid phase and within each group the different molecular species are arranged in discrete elements along a second horizontal axis of the solid phase.

26. A method according to claim 1, wherein the immobilised molecules are present within discrete spatially addressable elements and each element comprises a distinct spatially addressable micro electrode or nano electrode.

27. A method according to claim 26 wherein said electrodes are formed of conducting polymers.

28. A method according to claim 27 wherein said electrodes are produced by a method selected from inkjet printing, soft lithography, nanoimprint lithography/lithographically induced self assembly, VLSI methods and electron beam writing.

29. A method according to claim 1, wherein the immobilised molecules are immobilised onto a single electrode.

30. A method according to claim 29 wherein the electrode(s) transduce a signal when a target molecule binds to an immobilised molecule present in the same element as an electrode.

31. A method for typing single nucleotide polymorphisms (SNPs) and mutations in nucleic acids, comprising the steps of:
a) providing a repertoire of probes complementary to one or more nucleic acids present in a sample, which nucleic acids may possess one or more polymorphisms, said repertoire being presented such that molecules in said repertoire may be individually resolved;
b) exposing the sample to the repertoire and allowing nucleic acids present in the sample to hybridise to the probes at a desired stringency and optionally to be processed by enzymes;
c) detecting individual hybridised nucleic acid molecules after optionally eluting the unhybridised nucleic acids from the repertoire.

32. A method according to claim 31, wherein the repertoire is arrayed on a solid phase.

33. A method according to claim 31, wherein the repertoire is arrayed at a density which allows molecules in said repertoire to be individually resolved.

34. A method according to claim 33, wherein said array is an array according to claim 31.

35. A method according to claim 31, wherein the sample is exposed to a second repertoire of probes, which probes bind to one or more molecules of the sample at a different position to the probes of the first repertoire.

36. A method according to claim 35, wherein said first and second repertoires are differentially labelled.

37. A method for determining the complete or partial sequence of a target nucleic acid, comprising the steps of:
a) providing a first set of probes complementary to one or more nucleic acids present in a sample, said first set of probes being presented such that arrayed molecules may be individually resolved;
b) hybridising a sample comprising a target nucleic acid to the first set of probes;
c) hybridising one or more further probes of defined sequence to the target nucleic acid; and
d) detecting the binding of individual further probes to the target nucleic acid. e) and detecting the approximate distance separating each probe or the order of each probe

38. A method according to claim 37, wherein the first set of probes is a repertoire of probes.

39. A method according to claim 38, wherein the repertoire is arrayed on a solid phase.

40. A method according to claim 39, wherein the target nucleic acids are captured to the solid phase at one or more points.

41. A method according to claim 37, wherein the repertoire is arrayed at a density which allows molecules in said repertoire to be individually resolved.

42. A method according to claim 37, wherein the probes are differentially labelled.

43. A method for determining the number of sequence repeats in a sample of nucleic acid, comprising the steps of:
a) providing one or more probes complementary to one or more nucleic acids present in a sample, which nucleic acids may possess one or more sequence repeats, said probes being complementary to a sequence flanking one end of the repeats, said probes being presented such that molecules may be individually resolved;
b) contacting the nucleic acids with labelled probes complementary to units of said sequence repeats and a differentially labelled probe complementary to the flanking sequence at the other end of the targeted repeats;
c) contacting the complex formed in b) with probes in a); and
d) determining the number of repeats present on each sample nucleic acid by individual assessment of the number of labels incorporated into each molecule and only counting those molecules to which the differentially labelled probe complementary to the flanking sequence is also associated with.

44. A method according to claim 43, wherein the repertoire is arrayed on a solid phase.

45. A method according to claim 43, wherein the repertoire is arrayed at a density which allows molecules in said repertoire to be individually resolved.

46. A method for analysing the expression of one or more genes in a sample, comprising the steps of:
a) providing a repertoire of probes complementary to one or more nucleic acids present in a sample, said repertoire being presented such that molecules may be individually resolved;
b) hybridising a sample comprising said nucleic acids to the probes; and
c) determining the nature and quantity of individual nucleic acid species present in the sample by counting single molecules which are hybridised to the probes.

47. A method according to claim 46, wherein the repertoire is arrayed on a solid phase.

48. A method according to claim 46, wherein the repertoire is arrayed at a density which allows molecules in said repertoire to be individually resolved.

49. A method according to claim 46, wherein the repertoire comprises a plurality of probes of each given specificity.

50. A method for typing single nucleotide polymorphisms (SNPs) and mutations in nucleic acids, comprising the steps of:
a) providing a repertoire of probes complementary to one or more nucleic acids present in a sample, which nucleic acids may possess one or more polymorphisms;
b) arraying said repertoire such that each probe in the repertoire is resolvable individually
c) exposing the sample to the repertoire and allowing nucleic acids present in the sample to hybridise to the probes at a desired stringency and optionally be processed by enzymes such that hybridised/processed nucleic acid/probe pairs are detectable;
d) eluting the unhybridised nucleic acids from the repertoire and detecting individual hybridised nucleic acid/probe pairs;
e) analysing the signal derived from step (d) and computing the confidence in each detection event to generate a PASS table of high-confidence results; and
f) displaying results from the PASS table to type polymorphisms present in the nucleic acid sample.

51. A method according to claim 50, wherein confidence in each detection event is computed in accordance with Table 1.

52. A method according to claim 50, wherein detection events are generated by labelling the sample nucleic acids and/or the probe molecules, and imaging said labels on the array using a detector.

53. A method according to claim 50, where probe and/or target acts as a primer or ligation substrate.

54. A method according to claim 50, wherein the probe and or target is enzymatically processed by ligases or polymerases or thermophilic varieties thereof.

55. A method according to claim 50, wherein the probe forms secondary structures which facilitate or stabilise hybridisation or improve mismatch discrimination.

56. A method for determining the sequence of all or part of a target nucleic acid molecule which method comprises:
(i) immobilising the target molecule to a solid phase at two or more points such that the molecule is substantially horizontal with respect to the surface of the solid phase;
(ii) straightening the target molecule, during or after immobilisation;
(iii) contacting the target molecule with a nucleic acid probe of known sequence; and
(iv) determining the position within the target molecule to which the probe hybridises.

57. A method according to claim 56 wherein the target molecule is contacted with a plurality of probes.

58. A method according to claim 57 wherein each probe is labelled with a different detectable label.

59. A method according to claim 57 wherein the target molecule is contacted sequentially with each of the plurality of probes.

60. A method according to claim 59 wherein each probe is removed from the target molecule prior to contacting the target molecule with a different probe.

61. A method according to claim 57 wherein the target molecule is contacted with all of the plurality of probes substantially simultaneously.

62. A method according to claim 60 wherein the probes are removed by heating, modifying the salt concentration or pH, or by applying an appropriately biased electric field.

63. A method according to claim 56 wherein the target is substantially a double stranded molecule and is probed by strand invasion using PNA or LNA.

64. A method according to claim 56 wherein the target nucleic acid molecule is a double-stranded molecule and is derived from a single-stranded nucleic acid molecule of interest by synthesising a complementary strand to said single-stranded nucleic acid.

65. A method for determining the sequence of all or part of a target single-stranded nucleic acid molecule which method comprises:
(i) immobilising the target molecule to a solid phase at two or more points such that the molecule is substantially horizontal with respect to the surface of the solid phase;
(ii) straightening the target molecule, during or after immobilisation
(iii) contacting the target molecule with a plurality of nucleic acid probes of known sequence, each probes being labelled with a different detectable label; and (iv) ligating bound probes to form a complementary strand.

66. A method according to claim 65 wherein prior to step (iv), any gaps between bound probes are filled by polymerisation primed by said bound probes.

67. A method according to claim 65 wherein the solid phase is a bead or particle.

68. A method according to claim 65 wherein the solid phase is a substantially flat surface.

69. A method for arraying a plurality of nucleic acid molecules which method comprises:
(i) immobilising the plurality of nucleic acid molecules randomly to a solid substrate;
(ii) optionally horizontalising and straightening the molecules, during or after immobilisation; and
(iii) contacting the plurality of nucleic acid molecules with a plurality of probes, each probe being labelled, such that each immobilised molecule can be identified uniquely by detecting the probes bound to the molecule.

70. A method according to claim 69 wherein the plurality of nucleic acid molecules are immobilised at a density such that individual immobilised molecules in the sample can be individually resolved.

71. A method for arraying a plurality of nucleic acid molecules which method comprises:
(i) contacting the plurality of nucleic acid molecules with a plurality of probes, each probe being labelled with a tag which indicates uniquely the identity of the probe, such that each molecule can be identified uniquely by detecting the probes bound to the molecule and determining the identity of the corresponding tags;
(ii) immobilising the plurality of nucleic acid molecules randomly to a solid substrate; and optionally
(iii) horizontalising and straightening the molecules, during or after immobilisation.

72. A method according to claim 71 wherein the plurality of nucleic acid molecules are immobilised at a density such that individual immobilised molecules in the sample can be individually resolved.

73. A method according to claim 69 or 71 wherein the solid phase is a substantially flat solid substrate or a bead/particle/rod/bar.

74. A method for producing a molecular array which method comprises immobilising to a solid phase a plurality of molecules present in a sample, wherein the plurality of molecules are immobilised at a density such that individual molecules in the sample can be individually resolved.

75. A method according to claim 74 wherein the plurality of molecules are polypeptides.

76. A method according to claim 74 wherein the plurality of molecules comprise the genome, proteome, transcriptome or metabolome of a cell, tissue or organism.

77. A method for identifying and/or characterising one or more molecules of a plurality of molecules present in a sample which method comprises:
(i) producing a molecular array by a method comprising immobilising to a solid phase a plurality of molecules present in a sample, wherein the plurality of molecules are immobilised at a density such that individual molecules in the sample can be individually resolved; and
(ii) identifying and/or characterising one or more molecule immobilised to the array.

78. A method according to claim 77 wherein step (ii) comprises contacting the array with a one or more probes and determining whether one or more of said probes interacts with one or more of said immobilised molecules.

79. A method according to claim 77 wherein one or more of said immobilised molecules is interrogated by an optical method.

80. A method according to claim 79 wherein the optical method is selected from far-field optical methods, near-field optical methods, epi-fluorescence spectroscopy, scanning confocal microscopy, two-photon microscopy and total internal reflection microscopy.

81. A method according to claim 78 wherein one or more of said immobilised molecules is interrogated by scanning probe microscopy or electron microscopy.

82. A method according to claim 77 wherein a physicochemical property of the immobilised molecules is determined, such as shape, size or mass, charge, hydrophobicity.

83. A method according to claim 77 wherein an electromagnetic, electrical, optoelectronic and/or electrochemical property of the immobilised molecules is determined.

84. A method according to claim 77 wherein a characteristic of a complex of between an immobilised molecule and a probe is determined

85. A method according to claim 77 wherein the plurality of molecules are polypeptides.

86. A method according to claim 77 wherein the plurality of molecules comprise the proteome, transcriptome or metabolome of a cell, tissue or organism.

87. A method according to claim 77 wherein the characteristics of individual immobilised molecules are learnt using a computational method.

88. A method according to claim 87 wherein the computational method is a neural network or artificial intelligence.

89. A molecular array obtained by the method of claim 87 wherein the characteristics of a plurality of immobilised molecules and their corresponding physical location in the array have been determined.

90. A multiplexed array comprising a plurality of arrays, each array comprising immobilized to a solid phase, a plurality of molecules at a density which allows individual immobilized molecules to be individually resolved, wherein each molecule in the array is spatially addressable and the identity of each molecule is known or determined prior to immobilization.

91. A method for identifying and/or characterising one or more molecules of a plurality of molecules present in a sample which method comprises:
(i) producing a molecular array by a method comprising immobilising to a solid phase a plurality of molecules present in a sample, wherein the plurality of molecules are immobilised at a density such that individual molecules in the sample can be individually resolved; and
(ii) identifying and/or characterising one or more molecule immobilised to the array by a method comprising contacting the immobilised molecules with a plurality of encoded probes.

92. A method according to claim 91 wherein each probes is encoded by virtue of being labelled with a tag which indicates uniquely the identity of the probe, such that an immobilised molecule can be identified uniquely by detecting the probes bound to the molecule and determining the identity of the corresponding tags.

93. A method according to claim 92 wherein the tagged probes are produced using combinatorial chemistry.

94. A method according to claim 92 wherein the tag is selected from a nanoparticle, a nanorod and a quantum dot.

95. A method according to claim 92 wherein each tag comprises multiple molecular species.

96. A method according to claim 92 wherein the tags are detectable by optical means.

97. A method according to claim 92 wherein the tags are particulate and comprise surface groups.

98. A method according to claim 92 wherein the tags are particulate and encase detectable entities, such as particle or molecules.

99. A method according to claim 92 wherein tags can be detected and distinguished by scanning probe microscopy.

100. A method according to claim 92 wherein the solid substrate is a bead/particle/rod/bar.

101. A method according to claim 92 wherein the solid phase comprises channels or capillaries within which the molecules are immobilised.

102. A method according to claim 92 wherein the solid phase comprises a gel.

103. A biosensor comprising a molecular array according comprising immobilized to a solid phase, a plurality of molecules at a density which allows individual immobilized molecules to be individually resolved, wherein each molecule in the array is spatially addressable and the identity of each molecule is known or determined prior to immobilization.

104. An integrated biosensor comprising a molecular array according to claim 103, an excitation source, a dectector, such as a CCD and optionally, signal processing means.

105. A biosensor according to claim 103 wherein the biosensor comprises a plurality of elements, each element containing distinct molecules, such as probe sequences.

106. A biosensor according to claim 105 wherein each element is specific for the detection of a different target, such as different pathogenic organisms.

107. A biosensor according to claim 103 wherein the molecular array is formed on an optical fibre.

108. A method according to claim 26, in which:
(a) the immobilised molecule is selectively coated with a material that facilitates detection
(b) the coating is a conducting material which allows a circuit to form between only those electrodes onto which are occupied by the target molecule by virtue of its binding to the alleic probe present on the electrode;
(c) a potential difference is applied between electrodes in any two contiguous groups of electrodes and the electrodes on which probes interact with target are identified by virue of the fact that a current flows between them;
(d) the conducting material comprises silver, gold, palladium or conjugated polymers; or
(e) multiple single molecules span the electrodes then the haplotype frequency is given by the amount of current that flows between the electrodes.

109. A method according to claim 69 in which the plurality of probes are labeled with a tag which indicates uniquely the identity of the probe.

110. A method according to claim 69 in which the plurality of tagged probes are hybridized substantially simultaneously or in groups of probes.

111. A method according to claim 1 in which probes are grouped according to their Tm.

112. A method according to claim 69, in which each of the plurality of labeled probes are successively hybridized to the immobilized nucleic acid and a record of those that hybridise to each molecule can be used to identify or re-assemble the sequence of the immobilized molecule.

113. A method according to claim 112 in which haplotype frequencies can be determined.

114. A method according to claim 70 in which probes are between lengths 3-9 mers.

115. An array comprising, immobilized to a solid phase, a plurality of molecules at a density which allows individual immobilized molecules to be individually resolved, wherein each molecule in the array is spatially addressable and the identity of each molecule is known or determined prior to immobilization.

116. A method of identifying one or more target molecules in a sample, comprising:
providing an array comprising a plurality of molecules immobilized to a solid phase at a density which allows individual immobilized molecules to be individually resolved, wherein each individual immobilized molecule in the array is spatially addressable and the identity of each immobilized molecule is known or encoded; and
contacting the array with said sample and interrogating one or more individual immobilized molecules to determine whether a target molecule has bound.


SEQUENCE LISTING (2 sequences)

SEQ ID NO: 1 - DNA, 12 bp, Artificial
  Lambda A overhang DNA
gggcggcgacct

SEQ ID NO: 2 - DNA, 12 bp, Artificial
  Lambda B overhang DNA
aggtcgccgccc